SEQUENCE LISTING (85 sequences)

SEQ ID NO: 1 - DNA, 1347 bp, Glycine max
atggtaaccattaaagcatcttacacagtccttccaaatgaaccaactccggagggccttttatggctatctgatattgatcaagtggcacgtctacgccacacacccaccatctacattttccatgcaaaacacaaccatgatacattaattgagaggatgagaaactctcttagcaagattttggttcactactatccaatagccggtcgattgagaaggatagaaggtagtggtcgattggaattggattgcaatgcaaaaggggtggtgttgcttgaagctgaatccacaaaaacattggatgattatggagactttttacgtgagtccatcaaggacttggttccaacggttgattacacaagccctatagaggaacttccctcgctactagttcaagtaacaacattccatggtggtaaatcctttgccattggagttgccttatgccatatcttgtgtgatggggttggtgccattcagtttatcaactcttgggcaaagttagctcgaggagacacattggagccacatgagatgccatttctggatcgaacagtgctcaagttcccacaccctctgtcgccgccgcgctttgatcacctggagttcaagcctttgccacttatattgggaagatctgacaacacagtagagaaaaacaagaaggttgatgccaccttgctgaagctaacaccagaacaagtggggaagctgaagaagaaggccaatgatgactcaacaaaagaagggtcaagaccttatagcagatttgaagctattgctgcacatatatggagatgtgcctctaaggctcgtaaactagataagaatcaaccaactctagttcggttcaatgccgatattcgcaatagactaattccacctctccctaagaattattttgggaatgctttgtccttaacaacagcatcatgtcacgttggagatgtcatatcaaattctttgagttatgctgctcagaagataagggaagcaattgaagtggttacatatgagtatatatggtcacagatagatgtgattagaggtcaggagcaactggataatgcaagggctttgttctttggacaaaatgaaggaaaggatgccttgttttatgggaaccctaaccttcttattacaagttggatgagcatgccaatgcacgaagcagattttggctggggaaagcctgtgtatttaggtctaggaagtgtatctacacaagatagggcactgattattcaaagccctgatggtgacggctctattattctgtccatacactttcagatggaacatatgcagcttttcaagaaatatttttatgaagatatgtga

SEQ ID NO: 2 - protein, 448 aa, Glycine max
MVTIKASYTVLPNEPTPEGLLWLSDIDQVARLRHTPTIYIFHAKHNHDTLIERMRNSLSKILVHYYPIAGRLRRIEGSGRLELDCNAKGVVLLEAESTKTLDDYGDFLRESIKDLVPTVDYTSPIEELPSLLVQVTTFHGGKSFAIGVALCHILCDGVGAIQFINSWAKLARGDTLEPHEMPFLDRTVLKFPHPLSPPRFDHLEFKPLPLILGRSDNTVEKNKKVDATLLKLTPEQVGKLKKKANDDSTKEGSRPYSRFEAIAAHIWRCASKARKLDKNQPTLVRFNADIRNRLIPPLPKNYFGNALSLTTASCHVGDVISNSLSYAAQKIREAIEVVTYEYIWSQIDVIRGQEQLDNARALFFGQNEGKDALFYGNPNLLITSWMSMPMHEADFGWGKPVYLGLGSVSTQDRALIIQSPDGDGSIILSIHFQMEHMQLFKKYFYEDM

SEQ ID NO: 3 - DNA, 1065 bp, Glycine max
atggctatgcatggaacttcttgtttggtgccttctgttctggagctagcaaaacaacccatcatcgaagttccagaacgatatgttcatgccaatcaagatccacatattctttccaacacaatttctttaccacaagttcccatcatagacttgcatcaattgttgtctgaagatccaagtgaactagagaagctggaccatgcgtgcaaagaatggggtttctttcagcttataaaccatggagtggaccctccagtggtggaaaatatgaagataggtgttcaagaattcttcaaccttccaatggaagagaagcagaaattttggcaaacaccagaggatatgcaggggtttggccagttatttgttgtatcagaggaacaaaagctagaatgggcagacatgttctatgctcacacatttccattacattcaaggaatcctcatcttattcctaagattccccaaccattcagagaaaatctagagaactattgcttagagctgagaaaaatgtgcatcacaatcattggccttatgaaaaaagctctcaagatcaaaacaaatgaactctcagagttatttgaagatcctagccaaggaattaggatgaattactaccctccatgtccccaaccagaacgagtcattggaatcaatccgcattctgattcgggtgcccttaccatccttctccaagtcaatgaagtggaagggcttcaaataagaaaagatggaaagtggattcctgttaaaccactctctaatgcttttgtcatcaacgttggagacatgttggagatactgaccaatgggatttaccgaagcattgaacatcggggaatagtcaattcagaaaaagagaggatttccattgccatgtttcaccggcctcaaatgagcagagttataggtccagcaccaagcctcgttactcctgaaagacctgcattgttcaaaaggattggagtagcagattacctcaatggattccttaaacgtgagctaaaaggaaaatcatacatggatgtcattaggatccaaaacgagattggtaaataa

SEQ ID NO: 4 - protein, 354 aa, Glycine max
MAMHGTSCLVPSVLELAKQPIIEVPERYVHANQDPHILSNTISLPQVPIIDLHQLLSEDPSELEKLDHACKEWGFFQLINHGVDPPVVENMKIGVQEFFNLPMEEKQKFWQTPEDMQGFGQLFVVSEEQKLEWADMFYAHTFPLHSRNPHLIPKIPQPFRENLENYCLELRKMCITIIGLMKKALKIKTNELSELFEDPSQGIRMNYYPPCPQPERVIGINPHSDSGALTILLQVNEVEGLQIRKDGKWIPVKPLSNAFVINVGDMLEILTNGIYRSIEHRGIVNSEKERISIAMFHRPQMSRVIGPAPSLVTPERPALFKRIGVADYLNGFLKRELKGKSYMDVIRIQNEIGK

SEQ ID NO: 5 - DNA, 1065 bp, Medicago truncatula
atggctatgaagggaacctctctcctggttccttctgttcaagagttagtaaaacaacccattaccaatattccagaacgatatcttcatccaaatcaagaccctattgctgtttctaacacaagctctttaccacaagttcctgttatcgacctgcataaattgttgtctgatgatgcaactgagctacaaaattttgaccatgcttgcagaggctggggtttctttcagcttattaatcatggagtgaacactccaattgttgaaaatatgaagataggtgttgaacaatttctcaaacttccaatggaagagaagaagaaattttggcaaacaccaaatgatgtgcaggggtttggtcagttgtttgttgtatctgatgaacaaaagctcgaatgggcagacatgttctacattaacactttccctccggatgcaaggcatccacatctaattcctaatattcccaaaccattcagagatcatctagagaactattgtttagaattgaaaaaactagctgtcacgataattggtcgtatggagaaagctctaaagataaaaagcaacgaacttgtcgagttttttgaagatatatatcaaggaatgaggatgaattactatcccccatgtccccagccagagcatgtcattggactcaaacctcattctgattctggtgcccttactatccttctccaagtcaatgaagtggaaggccttcaaatcagaaaagacggaatgtggattcctatcaagcccctctctgatgcttttgtcgtcaacattggagacatgttggagatattaactaacggaatttaccgaagcattgaacatcgagcaacaatcaattcaatgaatgagaggatatcaattgctacatttcatagggctcaaatgagcaaaattctaggtccaacaccagaccttattactgctgaaagacctgctttgttcaaaagaattagggcagttgattaccttaatggattcatgtcacgtgagctacaaggaaaatcatgtatagattttgtaaggattcaaaatgatattggtaaataa

SEQ ID NO: 6 - protein, 354 aa, Medicago truncatula
MAMKGTSLLVPSVQELVKQPITNIPERYLHPNQDPIAVSNTSSLPQVPVIDLHKLLSDDATELQNFDHACRGWGFFQLINHGVNTPIVENMKIGVEQFLKLPMEEKKKFWQTPNDVQGFGQLFVVSDEQKLEWADMFYINTFPPDARHPHLIPNIPKPFRDHLENYCLELKKLAVTIIGRMEKALKIKSNELVEFFEDIYQGMRMNYYPPCPQPEHVIGLKPHSDSGALTILLQVNEVEGLQIRKDGMWIPIKPLSDAFVVNIGDMLEILTNGIYRSIEHRATINSMNERISIATFHRAQMSKILGPTPDLITAERPALFKRIRAVDYLNGFMSRELQGKSCIDFVRIQNDIGK

SEQ ID NO: 7 - DNA, 1077 bp, Glycine max
atggacccggaaatagtaaagaagctaggaagctctcttctggtgccttctgtgcaggaattagcaaagcaaggcatcaccaaagtcccagaacgatatgttcgacccaatgaagacccatgtgttgaatatgacacaacatcgttgccacaagtcccagttatcgacctgagcaagttgttgtctgaagatgatgctgctgaactagagaagctggatcatgcgtgcaaagaatggggtttctttcagttgatcaatcacggagttaatccttgtttggtggaatatatgaagaaaaatgtgcaagagctcttcaatcttccacatgaagagaagaagttattgtggcagaaaccaggagaaatggagggatttggtcaaatgtttgttgtatcagaggaacacaaattagaatgggcagatttattctacatttccactcttccatcatacgcaagacacccccacttgtttcctaatattccccgacaattcagggataatctagagaaatattctttagaattgaaaaaactttgcatcctaatctttgaatttatgacaaaagctcttaagatccaacccaacgaattgctagatttttttgaagagggagggcaagcaatgaggatgaattattaccctccatgtccccaaccagaacaagtcattggacttaatcctcattctgatgctggtgcccttacaatccttctccaagtcaatgaaatggatggccttcaaataagaaaagatggaatgtggattcctatcaaacccctctctaatgcttttgtcatcaatgttggagacatgctggagataatgaccaatggtatttaccggagcattgaacataaagctacagttaactcagagaaggaaaggatttccgttgctacatttcatagtccaagattgaccgcagttataggtccagcacaaagcctcatcactcctgaaagacctgcaactttcaatagcattagtgtagaagatttcttcaaagggtacttttcacgcgagctgcaagggaagtcatacattgatgtcatgagaatccaaaatgggttatag

SEQ ID NO: 8 - protein, 358 aa, Glycine max
MDPEIVKKLGSSLLVPSVQELAKQGITKVPERYVRPNEDPCVEYDTTSLPQVPVIDLSKLLSEDDAAELEKLDHACKEWGFFQLINHGVNPCLVEYMKKNVQELFNLPHEEKKLLWQKPGEMEGFGQMFVVSEEHKLEWADLFYISTLPSYARHPHLFPNIPRQFRDNLEKYSLELKKLCILIFEFMTKALKIQPNELLDFFEEGGQAMRMNYYPPCPQPEQVIGLNPHSDAGALTILLQVNEMDGLQIRKDGMWIPIKPLSNAFVINVGDMLEIMTNGIYRSIEHKATVNSEKERISVATFHSPRLTAVIGPAQSLITPERPATFNSISVEDFFKGYFSRELQGKSYIDVMRIQNGL

SEQ ID NO: 9 - DNA, 1065 bp, Glycine max
atggattcccaaatgacgaagcttggaacctctctattggttccttctgttcacgagctagctaagcaacccatgaccaaagttccagaacgatatgttcgtctaaaccaagatcctgttgtatctgacacaatctctctaccacaagtcccagtcattgatctgaacaaattgttttctgaagatggaactgaggtagagaagctaaaccaagcatgcaaagaatggggtttcttccagcttataaatcatggggtcaaccctttattggtgcaaaatgtgaagataggtgttcaagagttcttcggtcttcaaatggaagagaagaggaaactttggcaaaaacaaggagagttggaggggtatggtcaaatgtttgttgtatctgaggaacaaaagttagaatgggcagatatattctacattaatacgctaccatcatgcgcaaggaatccccacatatttgctagtattccccaaccattcagaaatgatctagagagctattctttagaattgggaaaacttagcatcgcaatcattaaacttatttcaaaagctcttgagatcaacactaatgaattgctagagttatttgaagatttaagtcagtcaatgaggatgaattgctaccctccatgtccccaaccagaacatgtcattggccttaatcctcattctgatgctggtgctctcaccatcctcctccaagtcaacgaaatggaaggcctgcaaataaggaaagatggaatgtggattcctattaaacctctctctaatgcttttgtcatcaatgttggagatattctggagatattgactaatggaatttatagaagcgttgaacaccgagcaacaatcaacgcagagaaggagagaatttctattgctacatttcacaggcctcaaatgaacagaattgtaggtccaacgccaagccttgttactcctgaaaggcctgcactgtttaaaagaattggtgtagcagattactatagaggctatttttcacgcgagttacgaggaaaatcatacattgatgttattaaaataaaaaatacatag

SEQ ID NO: 10 - protein, 354 aa, Glycine max
MDSQMTKLGTSLLVPSVHELAKQPMTKVPERYVRLNQDPVVSDTISLPQVPVIDLNKLFSEDGTEVEKLNQACKEWGFFQLINHGVNPLLVQNVKIGVQEFFGLQMEEKRKLWQKQGELEGYGQMFVVSEEQKLEWADIFYINTLPSCARNPHIFASIPQPFRNDLESYSLELGKLSIAIIKLISKALEINTNELLELFEDLSQSMRMNCYPPCPQPEHVIGLNPHSDAGALTILLQVNEMEGLQIRKDGMWIPIKPLSNAFVINVGDILEILTNGIYRSVEHRATINAEKERISIATFHRPQMNRIVGPTPSLVTPERPALFKRIGVADYYRGYFSRELRGKSYIDVIKIKNT

SEQ ID NO: 11 - DNA, 1065 bp, Medicago truncatula
atgtctaacttaggaacctcattattggttccttatgtccaagagctcgcaaagcagcccaacatagaagttccagaacaatatcttcaaccaaatcaagaccctattaatgtatcaaatacatcttccttacaacaagttccagtcattgacctaaataaattgttatcagaagatgcaactgagctagaaaaccttgatcaagcatgtaaagaatggggattcttccagttgattaatcacggagtcgatcatttattggtggaaaatgtgaagataggggttcaagagttcttaagtcttccggtggaagagaagaagaaattacggcaaactccagaagatatgcaggggtttggtcaattgtttgttgtatccgagaatcaaaagttagaatgggcggatttattctacactacaactcttccttcttacgcaaggaatacgcgcttatttccgaatattcctcaaccattcagagataatttagagacctattgtttagaattgcaaaatgtttgcatcacaatcataaaacatatggcaaaagctcttaaagttgaaccaaatgaaatactcgagttatttgaagacggaggtcaacaaatgaggatgaattactatcctccttgtccccaaccagaaaatgtcattggactcaatcctcattctgatgcgggtgccttaaccatccttctacaagccaatgacatcgaaggtctccaaatacgaaaagatggacagtggatttctgttaaaccactcaccgatgcttttgtcataaacgttggagacattttggagataatcaccaacggaatttatcggagtatcgaacatcgagcaacagttaactcggagaaagagaggatttccgtagcagcatttcacagaccccaaataagcaaagttataggtccaacaccaacacttgttactcctgaaaggcctgcattattcaaaaaacttactgtggaagattactgcaaagcattcttttcacgtaagctacaaggaaagtcatgccttgatattatgagaatccaaaatgagaatgatatgtga

SEQ ID NO: 12 - protein, 354 aa, Medicago truncatula
MSNLGTSLLVPYVQELAKQPNIEVPEQYLQPNQDPINVSNTSSLQQVPVIDLNKLLSEDATELENLDQACKEWGFFQLINHGVDHLLVENVKIGVQEFLSLPVEEKKKLRQTPEDMQGFGQLFVVSENQKLEWADLFYTTTLPSYARNTRLFPNIPQPFRDNLETYCLELQNVCITIIKHMAKALKVEPNEILELFEDGGQQMRMNYYPPCPQPENVIGLNPHSDAGALTILLQANDIEGLQIRKDGQWISVKPLTDAFVINVGDILEIITNGIYRSIEHRATVNSEKERISVAAFHRPQISKVIGPTPTLVTPERPALFKKLTVEDYCKAFFSRKLQGKSCLDIMRIQNENDM

SEQ ID NO: 13 - DNA, 1053 bp, Glycine max
atgtccaagtatggaacatctctattggtgccttctgttcatgaacttgtgaagcaacctatcaccaaagttccagatcaatatcttcatccaaatcaagatcctcctgatatatctaacacaaccttaccacaagttccagtcatcgaccttagtaaattgttatctgaagatgtcactgagctagagaagctagacgatgcatgcaaggaatgggggttcttccagctgattaatcatggagtcaacccttcaatggtggaaaacgtgaagagagatgttcaagagtttttaaatcttccaatggaaaagaagaagcaattttggcaaatcccagatgaattggagggttttggccagttgtttgttgtatctgaggatcaaaagctagaatgggcagatatgttcttcattcacactcttccaataaacgcaaggaatcttcgcttatttcctaactttcctcaaccattgagggataatatagagaactattcttcgcaattgaaaaagctttgcctcacaatcattgagcgtatggcgatggctcttaagattgaatcgaatgaactgctagattacgtgtttgaagatgtatttcaaacaatgagatggacttactaccctccatgccctcaaccagaaaatgtcattggaataaatcctcattcagatgcttgtgctcttaccattctactccaagccaatgaaacagaaggcctacaaataaaaaaagatggaaattggatccctgtcaaacccttgcctaatgcttttgtcatcaacgttggagacattttggagatattgaccaatgggatttaccggagcatcgagcatcgagcaactattaacaaagagaaagagagaatttccgttgccacatttcacaggcctctaatgaacaaagttataggtccaacacccagccttgttacttctgaaagagctgcagtgttcaaaagaattgctgttgaagattattacaaagcatacttttctcgcgggctaaaaggaaaatcatgccttgatttgatcagggtccaaaaatga

SEQ ID NO: 14 - protein, 350 aa, Glycine max
MSKYGTSLLVPSVHELVKQPITKVPDQYLHPNQDPPDISNTTLPQVPVIDLSKLLSEDVTELEKLDDACKEWGFFQLINHGVNPSMVENVKRDVQEFLNLPMEKKKQFWQIPDELEGFGQLFVVSEDQKLEWADMFFIHTLPINARNLRLFPNFPQPLRDNIENYSSQLKKLCLTIIERMAMALKIESNELLDYVFEDVFQTMRWTYYPPCPQPENVIGINPHSDACALTILLQANETEGLQIKKDGNWIPVKPLPNAFVINVGDILEILTNGIYRSIEHRATINKEKERISVATFHRPLMNKVIGPTPSLVTSERAAVFKRIAVEDYYKAYFSRGLKGKSCLDLIRVQK

SEQ ID NO: 15 - DNA, 453 bp, Medicago truncatula
atggacagaaacaattctcgcagacaacttgctgcaatgaaacagtccctctttgatcaaggacatcttgatgaacagtttattcaacttgaagaattgcaagatgatgctaatcctaattttgctgaggaaattgtcactctttactaccgcgattcatctaggcttatctctagctcggaacaaacactggagaggagtccactggatttcaacaagctggacacaatcatgcaccagtttaaaggaagcagctcaagcattggggccaaaaaggtgaaagcagaatcaactctaattagggaatattgcaggacacgaaatgcagaacgatgcaggaagagctttcaacaaatgaagaaagaatatgtagcattgagaaagagacttgaaacttattttcaattggctaggcaagctgggccaatggatagagcatgtcgccctaagtaa

SEQ ID NO: 16 - protein, 150 aa, Medicago truncatula
MDRNNSRRQLAAMKQSLFDQGHLDEQFIQLEELQDDANPNFAEEIVTLYYRDSSRLISSSEQTLERSPLDFNKLDTIMHQFKGSSSSIGAKKVKAESTLIREYCRTRNAERCRKSFQQMKKEYVALRKRLETYFQLARQAGPMDRACRPK

SEQ ID NO: 17 - DNA, 453 bp, Medicago truncatula
atggacagaaacaattctcgcagacaacttgctgcaatgaaacagtccctctttgatcaaggacatcttgatgaacagtttattcaacttgaagaattgcaagatgatgctaatcctaattttgttgaggaacttgtcactctttactaccgtgattcatctaggcttatctctagcttggaacaaacactggagaggagtccactggatttcaacaagctggacacaatcatgcaccagtttaaaggaagcagctcaagcattggggccaaaaaggtgaaagcagaatcaactctaattagggaatattgcaggacaggaaatgcagaaggatgcaggaagagctttcaacaaatgaagaaagaatatgtagcattgagaaagagacttgaaacttattttcaattggctaggcaagctgggccaatggatagagcatgtcgccctaagtaa

SEQ ID NO: 18 - protein, 150 aa, Medicago truncatula
MDRNNSRRQLAAMKQSLFDQGHLDEQFIQLEELQDDANPNFVEELVTLYYRDSSRLISSLEQTLERSPLDFNKLDTIMHQFKGSSSSIGAKKVKAESTLIREYCRTGNAEGCRKSFQQMKKEYVALRKRLETYFQLARQAGPMDRACRPK

SEQ ID NO: 19 - DNA, 456 bp, Glycine max
atggacagaaaccaatcccgcaggcaggttgccgcaatgaaacagtccctctttgatcaggggttcctggacgaacagtttatccaactggaagaattgcaggatgatgcaaatcctaactttgttgaggaaatcgtgactcttcactaccgtgattcgtcacggcttatctcaagcatagagcaggctctcaaagagaggaaccctctggatttcaacaagctggacacacttatgcatcagttcaaaggaagcagttcaagcataggagccaaaaaggtgaaagcagagtgcaatctgttcagggaatattgcaggacaggaaatgcagaaggatgcatgaggagcttccaacaactgaagagagaatatgctgcactgagaaagaaacttgaagcttattttcagttggctaggcaagctggaccccaggaaacagcatgtcgctccaaatga

SEQ ID NO: 20 - protein, 151 aa, Glycine max
MDRNQSRRQVAAMKQSLFDQGFLDEQFIQLEELQDDANPNFVEEIVTLHYRDSSRLISSIEQALKERNPLDFNKLDTLMHQFKGSSSSIGAKKVKAECNLFREYCRTGNAEGCMRSFQQLKREYAALRKKLEAYFQLARQAGPQETACRSK

SEQ ID NO: 21 - DNA, 456 bp, Glycine max
atggacagaaaccaatcccgcaggcaggttgctgcaatgaaacagtccctctttgatcaggggttacttgatgaacagtttatccaactggaagaattgcaggatgatgcaaatcctaactttgttgaagaaattgtgactcttcattaccgtgattcgtcgcggcttatctcaagcatagagcaggctctcaaagagaggaaccctctggatttcaacaagctggacacacttatgcatcagttcaaaggaagcagctcaagcataggagccaaaaaggtgaaaacagagtgtaatctgttcagagaatattgcagggcaggaaatgcagaaggatgcatgaggagcttccaacaactgaagagagaatatgcagcactgagaaagaaacttgaagcttattttcaggtgactagacaagctggaccccaggaaacagcatgtcgctccaaatga

SEQ ID NO: 22 - protein, 151 aa, Glycine max
MDRNQSRRQVAAMKQSLFDQGLLDEQFIQLEELQDDANPNFVEEIVTLHYRDSSRLISSIEQALKERNPLDFNKLDTLMHQFKGSSSSIGAKKVKTECNLFREYCRAGNAEGCMRSFQQLKREYAALRKKLEAYFQVTRQAGPQETACRSK

SEQ ID NO: 23 - DNA, 453 bp, Vigna unguiculata
atggacaaaatccattccctcaggcaagtagctgctatcaaacactccctcttcgatcagggatatttggatgagcaatttatccaactggaagaattgcaggatgatgctaatcctaactttgttgaggaaattgttactctttactatcgtgattcctctaggcttatcacaaacttggatcaaacactggaaaggaaccctctggatttcaacaaactggacacaattatgcaccagtttaaaggaagcagttcaagcattggagcaaaaaaggtggaagcagaatgcactctgtttagggaatattgcagggcaagaaatggagaaggatgcaggaggagcttccaacaaatgaagaaagaatatgcaacactgagaaaaaagcttgaaacatattttcagatggcaaggcaagctgggcccaaagagacagcatttcggcccaagtaa

SEQ ID NO: 24 - protein, 150 aa, Vigna unguiculata
MDKIHSLRQVAAIKHSLFDQGYLDEQFIQLEELQDDANPNFVEEIVTLYYRDSSRLITNLDQTLERNPLDFNKLDTIMHQFKGSSSSIGAKKVEAECTLFREYCRARNGEGCRRSFQQMKKEYATLRKKLETYFQMARQAGPKETAFRPK

SEQ ID NO: 25 - DNA, 675 bp, Populus trichocarpa
atggagagaaaccagttgcgtaggcaggttgctctcacaaggcagtctctttttgatcagggatttcttgatgaacaatttatccagcttgaggaactccaagatgatgcaaaccctaactttgtggaggaagtagtctcattgcactacagggattcagctagacttatcagtaacatagagaaagcattggagaagaatcctcttgactttaataagttggatggctatatgcatcaattcaagggaagcagctcaagcattggggccaaaaaggtgaaagctgagtgcactctctttagggaatattgcaaggcaggaaatggagaaggatgcatgaggacattccaacaaatcaagaaagaatatgcaacgctgaaaaggaagcttgagacttattttcagttagcaaggcaagccgggcctgctgacactgcatgtcggcccaattgtgtcaaatatgggtatggtttatttccaagctggactctagtccaggctcagttttgttctcaagtcccagcactcagcagggatcgcaaaaagcctaacgccggcctatatccagaagaaagcctatattcggtgggaatggagtgctggtcctaccggagctatacatccaatctactctggattcagtggaatatctctcaatcaataataagaaattaa

SEQ ID NO: 26 - protein, 224 aa, Populus trichocarpa
MERNQLRRQVALTRQSLFDQGFLDEQFIQLEELQDDANPNFVEEVVSLHYRDSARLISNIEKALEKNPLDFNKLDGYMHQFKGSSSSIGAKKVKAECTLFREYCKAGNGEGCMRTFQQIKKEYATLKRKLETYFQLARQAGPADTACRPNCVKYGYGLFPSWTLVQAQFCSQVPALSRDRKKPNAGLYPEESLYSVGMECWSYRSYTSNLLWIQWNISQSIIRN

SEQ ID NO: 27 - DNA, 1593 bp, Glycine max
atggactcttcttcttcatcacctccaaacagcaccaacaacaactccctcgctttctctctttccaatcactttcccaacccttcttcctctcccctttctctcttccactccttcacctatccatctctctctctcacaggcagcaacacggtggacgcaccgcctgagcccaccgctggagcaggaccgaccaacctctccatattcaccggcggccccaagttcgaggactttctgggcggttccgccgcaacagccaccaccgtcgcgtgtgcaccgccacagcttccgcagttctccaccgacaacaacaaccacctatacgattcggagctgaagtcaacaatagccgcgtgcttccctcgcgccttggccgccgaacaaagcaccgaaccgcaaaaaccatcccccaagaaaaccgtcgacaccttcgggcaacgcacctccatctaccgcggcgtgacccgacatagatggactgggagatacgaagctcatctatgggacaatagttgcagaagggaaggtcaaagcaggaaaggaaggcaagtttacttgggtggttatgacaaggaggataaggcagccagagcttatgatctcgcagctctcaagtactggggtccaactaccaccactaactttcctatttccaactatgagaaggaactggaggagatgaagaacatgactaggcaagagtttgttgcttctcttcgtaggaagagcagtggtttctctagaggggcctctatatacagaggagtaacgagacaccaccagcatggccgatggcaggcgagaataggcagagttgccggaaacaaagacctctaccttggcactttcagcacccaagaagaagctgctgaggcctatgacattgctgctatcaaattcaggggattaaatgcagtaacaaactttgacatgagtcgctacgacgtgaagagcattgcaaatagtactcttcctattggtggtttatctggcaagaacaagaactccacagattctgcatctgagagcaaaagccatgagccaagccaatccgatggagatccatcatcggcttcatcggtgacctttgcatcacagcaacaaccttcaagctccaacttaagctttgccatacccattaagcaagacccttcagattactggtccatcttggggtaccataatactccccttgacaacagtggcatcaggaacactactagtactgttactacaactacttttccatcctccaacaatggcactgctagtagtttgacacccttcaacatggagttctcaagtgccccctcaagtaccggcagcgataacaatgccgcgtttttcagtggaggaggcatctttgttcagcaacaaactagtcatggtcatggaaatgcaagcagtggttcctcctcttcttctttaagctgttcaatcccattcgccacgcccatattttctctaaatagcaatactagttatgagagcagtgctggttatggaaactggattggacctaccctgcacacattccaatcccatgcaaaaccaagtctctttcaaacgccaatatttggaatggaatga

SEQ ID NO: 28 - protein, 530 aa, Glycine max
MDSSSSSPPNSTNNNSLAFSLSNHFPNPSSSPLSLFHSFTYPSLSLTGSNTVDAPPEPTAGAGPTNLSIFTGGPKFEDFLGGSAATATTVACAPPQLPQFSTDNNNHLYDSELKSTIAACFPRALAAEQSTEPQKPSPKKTVDTFGQRTSIYRGVTRHRWTGRYEAHLWDNSCRREGQSRKGRQVYLGGYDKEDKAARAYDLAALKYWGPTTTTNFPISNYEKELEEMKNMTRQEFVASLRRKSSGFSRGASIYRGVTRHHQHGRWQARIGRVAGNKDLYLGTFSTQEEAAEAYDIAAIKFRGLNAVTNFDMSRYDVKSIANSTLPIGGLSGKNKNSTDSASESKSHEPSQSDGDPSSASSVTFASQQQPSSSNLSFAIPIKQDPSDYWSILGYHNTPLDNSGIRNTTSTVTTTTFPSSNNGTASSLTPFNMEFSSAPSSTGSDNNAAFFSGGGIFVQQQTSHGHGNASSGSSSSSLSCSIPFATPIFSLNSNTSYESSAGYGNWIGPTLHTFQSHAKPSLFQTPIFGME

SEQ ID NO: 29 - DNA, 1530 bp, Medicago truncatula
atggacaaatcctcttcttcacctcctacaaacactaacaacacctccctcgctttctctctttccaataataactttcctaatccttcacactcttcttcctctcacctctctcttttccactccttcacaccttatccttcctctataataccaccatctctcaccctcacaggaagcaacaaccccgtggaagcttcaccagaggcaacagacggaggaaccaccaacctctccatattcaccggcggccacaagtttgaggacttcttaggaagctccgttgcaccaacaagaaccgctgccgcaacgtgtgcaccgacgcagcttcagcagttttccaccgacaatgatgtatataattctgagctcaagaaaacaatagccgcttgctttcctggtggctaccccaccgaaccaaactccgaacctcaaaagccttctcctaaaaaaactgtcgataccttcggacaacgcacctccatctatcgcggtgtcacgagacatagatggacggggagatatgaagcacatctatgggacaatagttgcagaagggaaggacaaagtaggaaaggaagacaaggtggctatgataaggaagagaaagcagctagagcttatgatcttgctgctctcaagtactggggtccaacgaccactaccaactttccaatttccaactatgagaaggagattgatgacatgaagaacatgactagacaagaatttgttgcttctctacgaaggaagagtagtggtttctcaagaggagcatcaatttacagaggggtaacaagacaccaccagcatggccgatggcaggcaagaataggcagagttgccggaaacaaagacctctaccttggcactttcagcactcaagaagaagctgcagaagcgtatgatattgcagcaatcaaatttagaggactaaatgcagtaacaaattttgacatgagtcggtacgacgtaaagagtattgcaaattgttctcttcccataggaggtttatcaaacaagaacaataaaaattccacagattgtgtatcagaaaccaaaatcaatgaaccaattcaatccgacgaaatagatcatccatcttcaacttcgtcagcaacaactttaagcttcgccttacctattaaacaagacccttctaccgattattggtccaatattctcggcttccataataatcctagtgctgttactaccactaccataccattcaacatggatttttctgctcatgtaccttcaaataccaacagtgataatccccacaatgcagcatttttcagtggatcaggtatatttgttcagcaacaaaatatgaacggaagcagtggttccaattcttcatcttcgtcgtcagcttcaacgagttcaattccattcgcaacgccaatattttctttaaatagtaatagtagtagttatggaaatggaaataactggattggacacacattccaaacacatgcaaagccaagtctatttcaaacgccaatatttggaatggaatga

SEQ ID NO: 30 - protein, 509 aa, Medicago truncatula
MDKSSSSPPTNTNNTSLAFSLSNNNFPNPSHSSSSHLSLFHSFTPYPSSIIPPSLTLTGSNNPVEASPEATDGGTTNLSIFTGGHKFEDFLGSSVAPTRTAAATCAPTQLQQFSTDNDVYNSELKKTIAACFPGGYPTEPNSEPQKPSPKKTVDTFGQRTSIYRGVTRHRWTGRYEAHLWDNSCRREGQSRKGRQGGYDKEEKAARAYDLAALKYWGPTTTTNFPISNYEKEIDDMKNMTRQEFVASLRRKSSGFSRGASIYRGVTRHHQHGRWQARIGRVAGNKDLYLGTFSTQEEAAEAYDIAAIKFRGLNAVTNFDMSRYDVKSIANCSLPIGGLSNKNNKNSTDCVSETKINEPIQSDEIDHPSSTSSATTLSFALPIKQDPSTDYWSNILGFHNNPSAVTTTTIPFNMDFSAHVPSNTNSDNPHNAAFFSGSGIFVQQQNMNGSSGSNSSSSSSASTSSIPFATPIFSLNSNSSSYGNGNNWIGHTFQTHAKPSLFQTPIFGME

SEQ ID NO: 31 - DNA, 1629 bp, Populus trichocarpa
atggattctagttctcatcagaactggctaggtttctctctttccaaccatcatcacatgaataataccatcaacatccctacctcttctgattcctctcacctctgtctcttcgaagcctttaacaccaccactacctcagcacaagaagtaaacgcagtggtggctgctggtagagctacagacatctctttatttacagcttctggaccaaaacttgaggactttcttggtggttgcacctctacatcaccatcacaaacaccacaacagcagccactgtgtggtcagttctctactgagacacccgtgactaccaccgctactgctttatctgacagtactagttctgagatatatgactccgagctcaaaactatagctgctagcttccttcgtggttttgcctccactgatcatcaaaaaatcgatagtacccaaaaacatcaacaacttcttgttcaggccgaacatgcaccaaagaaaacagttgaaacttttggccaacgtacttcaatctatcgaggagtcaccaggcatagatggactggcagatatgaagcccatttgtgggataatagttgcagaagggaagggcagagtagaaaaggaagacaaggtggctatgacaaggaggagaaagcagccagagcttatgatcttgcagctctcaagtactggggtccgaccaccacaacaaactttccggtttctaactatgagaaggaaatagaagggatgaagcacatgacaaggcaagagtttgttgcgtcacttcgaagaaaaagtagtggtttctctagaggagcatcgatttacagaggagtgacgaggcaccatcaacacggtcggtggcaagcaaggatcggaagagttgctggcaacaaagatctctatcttggcacctttagcacgcaggaggaagctgccgaagcttatgacattgcagcgatcaaatttagaggcctaaatgctgtaaccaactttgacatgagtcgctatgacgtaaaaaacatagctaatagcaatcttcccattggaggaatatccggcaagtcaaaaaattcctcagaatccgcttctgatagcaaaagcattgatggaagccgatcggacgatcgagatctctcctcagcatcctcggtaacctttgcttctcagcctgcaacttctactcttagctttgccatacccatcaaacaagacccgtcagattattggaccaatatccttggataccaaaacaccactactatgaacaatgccaagaacagtagcagcagcattgttgatccaagcactttacttcaatcttccacaagcggtcctgcttttcaaagcccaacagttttcaaaatggacttcaatgcaaattcatctgtcaatgaaagcaataacaatgggctattattcaatggtggttatacacagcagcagataagtggtattggtacatcatcaccaagttcaaatatcccatttgctacacccatcgccttccatagtaatggtaatagttacgagggcaacccaagttatagtagctggattgctcaacctttgcattctttccaatctgcaaagcccaaactctcagtgtatcagactcccatttttggaattgaatga

SEQ ID NO: 32 - protein, 542 aa, Populus trichocarpa
MDSSSHQNWLGFSLSNHHHMNNTINIPTSSDSSHLCLFEAFNTTTTSAQEVNAVVAAGRATDISLFTASGPKLEDFLGGCTSTSPSQTPQQQPLCGQFSTETPVTTTATALSDSTSSEIYDSELKTIAASFLRGFASTDHQKIDSTQKHQQLLVQAEHAPKKTVETFGQRTSIYRGVTRHRWTGRYEAHLWDNSCRREGQSRKGRQGGYDKEEKAARAYDLAALKYWGPTTTTNFPVSNYEKEIEGMKHMTRQEFVASLRRKSSGFSRGASIYRGVTRHHQHGRWQARIGRVAGNKDLYLGTFSTQEEAAEAYDIAAIKFRGLNAVTNFDMSRYDVKNIANSNLPIGGISGKSKNSSESASDSKSIDGSRSDDRDLSSASSVTFASQPATSTLSFAIPIKQDPSDYWTNILGYQNTTTMNNAKNSSSSIVDPSTLLQSSTSGPAFQSPTVFKMDFNANSSVNESNNNGLLFNGGYTQQQISGIGTSSPSSNIPFATPIAFHSNGNSYEGNPSYSSWIAQPLHSFQSAKPKLSVYQTPIFGIE

SEQ ID NO: 33 - DNA, 1623 bp, Populus trichocarpa
atggattctacttctcatcagaactggctcggtttctctctttccaaccatcatcacatgaataatattaacatcccttcctcttctgattcctctaacctctgtctctttgaagcctttaacactacacccaccaccactacctcagcacaagaagataatgcagtagctgcctgtagaccaactgacatctctttatttaccacttctggaccaaaactagaggacttccttggttgttgcaccactacaccgccatcacaacagccactgggtggtcagttctcggctgagacacccgggactacggctactactgtatctgacaatagtagttctgagatatatgattctgagctcaaaactatagctgctagcttccttcgtggttattcctctaatgatcaccaacaaaccggtagttctcaaaaacatcaacagcttcttgtgcagtctgaacatgcaccaaagaaaactgttgatacttttggccaacgtacttcaatttatcgaggagtcaccaggcatagatggactggtagatatgaagcccatttgtgggataatagttgcagaagggaaggccaaagtagaaaaggaaggcaaggtggctatgacaaggaggagaaagcagctagagcttatgatcttgcagctctcaagtactggggtccgaccaccactacaaactttccggtttctaactatgagaaggaaatagaagggatgaagcacatgacaaggcaagagtttgttgcgtcacttcgaagaaaaagtagtggtttctctagaggagcatcgatttacagaggagtgacgaggcaccatcaacacggtcggtggcaagcaaggatcggaagagttgctggcaacaaagatctctatcttggcacctttagcacgcaggaggaagctgccgaagcttatgacattgcagcgatcaaatttagaggcctaaatgctgtaaccaactttgacatgagtcgctatgacgtaaaaagcatagctaacagcaatcttcccattggaggaatatctggcaaatcaaagaattcatcagaatcagtttctgatagcaaaagcattgatgtaagccgatcggacgatcgggatctctcctcggcgtcctcggtaacctttgcttctcagcctacaacctctactcttagctttgccatgcccatcaaacaagacccatcagattattggacaaatatccttggataccaaaactccaccaccacgatgaataataccaagaacagtagcagcattgtcgcaccaagcactttacttcaatcttccacaagttttcatgcttttcaaggtccaacagctttcagtatggacttcaacacgaattcttcagtcaatgaaagcaacaacagtgggctattattcaatggtggttatatacagcaacagagtggtggtgatggtattagtacatcatcatcaagttcgagtattccatttgctacacccattgccttgcatggtaatggaagcagttacgagggtaactcaagttatggcagctggatttctcaatctctgcattctttccaatctgcaaagcccaacctctcagtgtaccagacacctatttttggaatggaatga

SEQ ID NO: 34 - protein, 540 aa, Populus trichocarpa
MDSTSHQNWLGFSLSNHHHMNNINIPSSSDSSNLCLFEAFNTTPTTTTSAQEDNAVAACRPTDISLFTTSGPKLEDFLGCCTTTPPSQQPLGGQFSAETPGTTATTVSDNSSSEIYDSELKTIAASFLRGYSSNDHQQTGSSQKHQQLLVQSEHAPKKTVDTFGQRTSIYRGVTRHRWTGRYEAHLWDNSCRREGQSRKGRQGGYDKEEKAARAYDLAALKYWGPTTTTNFPVSNYEKEIEGMKHMTRQEFVASLRRKSSGFSRGASIYRGVTRHHQHGRWQARIGRVAGNKDLYLGTFSTQEEAAEAYDIAAIKFRGLNAVTNFDMSRYDVKSIANSNLPIGGISGKSKNSSESVSDSKSIDVSRSDDRDLSSASSVTFASQPTTSTLSFAMPIKQDPSDYWTNILGYQNSTTTMNNTKNSSSIVAPSTLLQSSTSFHAFQGPTAFSMDFNTNSSVNESNNSGLLFNGGYIQQQSGGDGISTSSSSSSIPFATPIALHGNGSSYEGNSSYGSWISQSLHSFQSAKPNLSVYQTPIFGME

SEQ ID NO: 35 - DNA, 1506 bp, Vitis vinifera
atggcttctaatcacaattggctcgccttctctctctccaacaactctctcccttccgactcctcccaactctgccttttcgaagccctctccgcctcaacccatcacaatgggggtgttgatagagagcagggttttccggatcgtggtggcagtgacctcatcggcggccccaagctggaggacttcctcggtggaggtggaggtggaggtggagcgggtgcaggtctcgatcagttctcggctaaaactccggcgggtttgtctgatgctgagatatacgagtccgagctgaagaccatagctgcgagcttcttacgtgggttttcttcagagcaaagcgaggctcagaagcagctggcgctgacgccggagccgtcgccgaagaagtccgtcgacaccttcggccaacggacctccatttacagaggagttacacgacatagatggacagggagatatgaagcccatctctgggacaacagctgcagaagagaaggacagagccggaagggacgacaagtttatttgggtgggtatgataaggaagagaaggcagctagagcttatgatctcgccgctcttaagtactggggtccgaccaccaccactaacttccctgtttctaactatgagaaagaacttgagaatatgaagaacatgactaggcaagagtttgttgcttcactcagaaggaaaagctctggattctcgagaggagcctctatttacagaggagtcaccagacaccatcagcatgggagatggcaagccagaataggaagagttgctgggaacaaagatctctatcttggaacattcagcactcaagaagaagcagccgaagcctatgacattgctgcaatcaaattcagaggcctaaatgcagtaaccaacttcgacatgagccgatacgatgtcaaaagcattgccaacagcaatctccccatcggaggagcaataaccactggcaaaccaaaaacatctccctctgattctgcttccgatagtggaagtcgccggtctgaggagcaggttcaggttctctctggcccatcaaatacactcattttttccaaacccctcaagcaagatcaccatcaatcggttgattactggtccgtactggggtaccaggcctccacattgaacagtgttgtgaagaaccccatattggaatcctcctttccatttcagacttctagcaacttgaccatggatttctccacagcttctgcaggcttcctctgcaatggtggtgggtacattcaacaacaacagagcaacagtagtagcagcactgcttcaagttcaaactcaatcccatatgcaacacccgttaatggtttaaacagcagtactagctatgaggtctctgggtacaacagctgggctgctgcgactgctgccgctgctgctgctgctgctgcaccatctcttcattcattccaagctgcaaagcccagtctctcagtgttccaaaccccgatatttggcatggaatga

SEQ ID NO: 36 - protein, 501 aa, Vitis vinifera
MASNHNWLAFSLSNNSLPSDSSQLCLFEALSASTHHNGGVDREQGFPDRGGSDLIGGPKLEDFLGGGGGGGGAGAGLDQFSAKTPAGLSDAEIYESELKTIAASFLRGFSSEQSEAQKQLALTPEPSPKKSVDTFGQRTSIYRGVTRHRWTGRYEAHLWDNSCRREGQSRKGRQVYLGGYDKEEKAARAYDLAALKYWGPTTTTNFPVSNYEKELENMKNMTRQEFVASLRRKSSGFSRGASIYRGVTRHHQHGRWQARIGRVAGNKDLYLGTFSTQEEAAEAYDIAAIKFRGLNAVTNFDMSRYDVKSIANSNLPIGGAITTGKPKTSPSDSASDSGSRRSEEQVQVLSGPSNTLIFSKPLKQDHHQSVDYWSVLGYQASTLNSVVKNPILESSFPFQTSSNLTMDFSTASAGFLCNGGGYIQQQQSNSSSSTASSSNSIPYATPVNGLNSSTSYEVSGYNSWAAATAAAAAAAAAPSLHSFQAAKPSLSVFQTPIFGME

SEQ ID NO: 37 - DNA, 1446 bp, Glycine max
atggagctgctatggcacaacggccaggtcgtggtgcagagtcagaaccaacgttctctgagaaaacttccgccggtgaccaactcccacgacgcctctcccgccggtccttcaatgacgagagagattcgaccgctggtggagaatttcaatcagcatctcttcatgcacgaaggcgaaatggcttcgtggcttcactatcctatcgacgacgacgagcctgccttcatgcaaaccctcggccacacgtctcagctcacggagctccggccgatgtcggcgaatccacggcctccgattccgcccccgagaaggccggaacagaggacgccgaacttcgcgtacttctcgaggcacaacacgagagccgcggagccgagcgtgaaggccgccgcgagggaatccacggtggtggattcgtgcgatacggaggcggcggcttcgagagtttcggagacagtaaggagcgcggcggaaggaggtgccggcgttgcggcgccttcgacgagtgccggcggtggaaggagcacgatgatgtacgatttgacgatgacgtcatcgcctggcggttcgagtagctgcgacgagccggttcaggtagcggcggcggaggaggatcggaagcggaagggacgggaggcggaggaatgggagtgtcagagcgagttacaaattccctgtaccctggtatacgctaacgtgcgctgggtgagtgacgtgggtttacgtgagcactccccgcgctgctgcatttatttcggtgctgtggccttgtgctcttttggtacggtcatcactttcaccgtcgtggctgcgcacgtgcaagcaaagaagcaagtttgtggatcaacatctacaaagagatcccgtgctgccgaggtccataatctctctgagaggaggcgtcgcgatcggattaatgaaaagatgaaagctctgcaagaacttatacctcgttgcaataagtcggacaaagcttcaatgctggatgaagcaatttcgtacttgaagtcattgcagttacaagtgcagatgatgtccatgggatgtggaatggtacctgtgatgtttcctggaatccagcagtacatgccagcaatgggaatgggggttggaatgggtatgggtatggaaatgggaatgaacagacctgtaatgccgtttcccaatatgttacctggttcggctttgccagcggcaactgcagctgctgctcatttgggaccaagaatgcaagcagcaaatcagtcagataataatatggtcacgtcagctggtccacctgatccaaatcagtcacgtatcccaaacttcactgatccttatcaacaatatcttggtccccaccagatgcagttccaattaattcagaatcaggcaatgaaccaaccaaatgttagcaagccaagtaacaatgggggccctgcgaatccagaaaaccattag

SEQ ID NO: 38 - protein, 481 aa, Glycine max
MELLWHNGQVVVQSQNQRSLRKLPPVTNSHDASPAGPSMTREIRPLVENFNQHLFMHEGEMASWLHYPIDDDEPAFMQTLGHTSQLTELRPMSANPRPPIPPPRRPEQRTPNFAYFSRHNTRAAEPSVKAAARESTVVDSCDTEAAASRVSETVRSAAEGGAGVAAPSTSAGGGRSTMMYDLTMTSSPGGSSSCDEPVQVAAAEEDRKRKGREAEEWECQSELQIPCTLVYANVRWVSDVGLREHSPRCCIYFGAVALCSFGTVITFTVVAAHVQAKKQVCGSTSTKRSRAAEVHNLSERRRRDRINEKMKALQELIPRCNKSDKASMLDEAISYLKSLQLQVQMMSMGCGMVPVMFPGIQQYMPAMGMGVGMGMGMEMGMNRPVMPFPNMLPGSALPAATAAAAHLGPRMQAANQSDNNMVTSAGPPDPNQSRIPNFTDPYQQYLGPHQMQFQLIQNQAMNQPNVSKPSNNGGPANPENH

SEQ ID NO: 39 - DNA, 519 bp, Glycine max
atggatgatgttgatgagatgaagagtactaagaagggaaaagggaataagaaaggtggtgggctgataaccaaaacatgggagcgatgcaagtccataggaagaagccgcaaggaggcaagttctaattccctcaatactaatactaataccatgagaagcaaatcatggccaaaccgtaaccgggcggaaaacaagaacaagaacaaaaacagtactatcgtggcccccgaaggttgcttctcggtgtatgttggaccccaaatgcaaaggtttgtcatcaaaaccgagtacgcaaaccaccccttgttcaagatgttgctcgaagaagctgaatctgagtatggttacaacagccaaggccctttggcccttccctgccacgttgatgtcttctacaaggtcttgatggaaatggattctgatgaaactcatggcagctgtgcctgtgttaagcgttctccctctgcttatcaacttcttcgcacttcccctatgctttcaataaaccacttctga

SEQ ID NO: 40 - protein, 172 aa, Glycine max
MDDVDEMKSTKKGKGNKKGGGLITKTWERCKSIGRSRKEASSNSLNTNTNTMRSKSWPNRNRAENKNKNKNSTIVAPEGCFSVYVGPQMQRFVIKTEYANHPLFKMLLEEAESEYGYNSQGPLALPCHVDVFYKVLMEMDSDETHGSCACVKRSPSAYQLLRTSPMLSINHF

SEQ ID NO: 41 - DNA, 1128 bp, Glycine max
atggttggcaaggagagtttgagaactcatgaggtcttgaactttagctttgagaagcttatgatggttgactgtggtggaaatagtgggaaaggagtgaactttaaagcgggggtgatcacagaatggaaggatattccagttgagcttttgatgcagattttgtcacttgtggatgatcaaacggttatgatagcttctgaagtttgtcgtgggtggagagaggcaatttgctttggcctgactcggttatcactctcatggtgtagcaagaacatgaataatttggtcctatccctagctcctaaattcacaaaattacagactttaatccttcgtcaagacaagcctcaactagaggacaatgctgttgaaactatttcaaatttttgtcatgacctccaaatcttggacctcagcaaaagtttcaagcttaccgatcattcgttgtatgccatagcccttggttgtcaggatcttacaaaactgaacatcagtggttgttcagcctttagtgacaatgctctggcttacctggccagtttttgtagaaagctgaaagttttgaatctctgtggatgtgttaaagctgcatctgatactgcgttacaggcaattgggcattactgcaatcagttacagtttttgaaccttggatggtgtgaaaatgtcagcgatgttggagtgatgagtttagcatatggctgccgtgatcttagaacactcgatttatgtggttgtgtccttataacagatgacagtgtaattgccttggcaaacagatgtcctcatctgaggtcccttggactttacttctgccaaaacatcacagacagggcaatgtattccttggcacaaagcaaagtgaacaacaggatgtggggttctatgaaaggtggtggaaataatgatgataatgatgatggactaagaactttgaacattagccaatgcacagcactcaccccttctgctgtgcaagctgtgtgtgactcatgcccttccctccacacctgttctggaagacactcactcatcatgagtggttgtctgaatctgacttctgtgcattgtgcttgtgctggccaagcacaccgtgctttcactctcccacatgcagctcattga

SEQ ID NO: 42 - protein, 375 aa, Glycine max
MVGKESLRTHEVLNFSFEKLMMVDCGGNSGKGVNFKAGVITEWKDIPVELLMQILSLVDDQTVMIASEVCRGWREAICFGLTRLSLSWCSKNMNNLVLSLAPKFTKLQTLILRQDKPQLEDNAVETISNFCHDLQILDLSKSFKLTDHSLYAIALGCQDLTKLNISGCSAFSDNALAYLASFCRKLKVLNLCGCVKAASDTALQAIGHYCNQLQFLNLGWCENVSDVGVMSLAYGCRDLRTLDLCGCVLITDDSVIALANRCPHLRSLGLYFCQNITDRAMYSLAQSKVNNRMWGSMKGGGNNDDNDDGLRTLNISQCTALTPSAVQAVCDSCPSLHTCSGRHSLIMSGCLNLTSVHCACAGQAHRAFTLPHAAH

SEQ ID NO: 43 - DNA, 1107 bp, Medicago truncatula
atggttgggaaggataatttaaagactgaggatttgaatttgttttttgagaatcttatgatggttgctggtggtgagaaaggagggaacatgaaggttggtgtgatcactgaatggaaggatattcctgttgagcttttgatgcagattctgtctcttgtggatgatcagactgtgattcgagtttctggtgtttgtcgtggttggagagattctatttactttggccttgctcgtttatcactctcttggtgtaacaagaacatgaacaacttggtcctatctcttgttcccaagtttgcaaaactgcaaactctaatccttcgtcaagataagccacaactggacgacaatgttgttggcactattgcaaatttctgtcatgacctgcagattcttgacctcagcaaaagtttcaagctcactgatcgctcactgtacgctatcgctcatggttgccgcgatcttacaaaactaaatatcagtggatgttcagcatttagtgacaatgctctggcctaccttgctggtttctgtagaaaactgaaagttctaaatctctgtggatgtgttagagcagcttctgatactgcacttcaggctattggacactactgcaatcagttgcagtctttgaaccttggatggtgtgacaaagttggtgatgttggagtgatgagtctagcatatggttgtcctgatctaaggacagttgatttgtgtggctgtgtctatataacagatgatagtgtgattgctttggcaaacggatgccctcacctgaggtcccttggactgtacttctgcaagaacataacagacaatgcaatgtattcattggcacaaagcaaagtgaagaacaggatgtggggatctgtgaaaggaggaaatgatgaagatggactaaggacattgaacatcagccagtgcacttcactcactccttcagctgttcaggcggtttgtgactcatctcctgctctccacacatgctcagggaggcattcactcatcatgagtggctgtctcaatttgaccgaagtccattgtgcgtgtgctggtcatgcacaccgtgcaatgaacgctttcccccatcctgctcattaa

SEQ ID NO: 44 - protein, 368 aa, Medicago truncatula
MVGKDNLKTEDLNLFFENLMMVAGGEKGGNMKVGVITEWKDIPVELLMQILSLVDDQTVIRVSGVCRGWRDSIYFGLARLSLSWCNKNMNNLVLSLVPKFAKLQTLILRQDKPQLDDNVVGTIANFCHDLQILDLSKSFKLTDRSLYAIAHGCRDLTKLNISGCSAFSDNALAYLAGFCRKLKVLNLCGCVRAASDTALQAIGHYCNQLQSLNLGWCDKVGDVGVMSLAYGCPDLRTVDLCGCVYITDDSVIALANGCPHLRSLGLYFCKNITDNAMYSLAQSKVKNRMWGSVKGGNDEDGLRTLNISQCTSLTPSAVQAVCDSSPALHTCSGRHSLIMSGCLNLTEVHCACAGHAHRAMNAFPHPAH

SEQ ID NO: 45 - DNA, 1107 bp, Medicago truncatula
atggttgggaaggataatttaaagactgaggatttgaatttgttttttgagaatcttatgatggttgctggtggtgagaaaggagggaacatgaaggttggtgtgatcactgaatggaaggatattcctgttgagcttttgatgcagattctgtctcttgtggatgatcagactgtgattcgagtttctggtgtttgtcgtggttggagagattctatttactttggccttgctcgtttatcactctcttggtgtaacaagaacatgaacaacttggtcctatctcttgttcccaagtttgcaaaactgcaaactctaatccttcgtcaagataagccacaactggacgacaatgttgttggcactattgcaaatttctgtcatgacctgcagattcttgacctcagcaaaagtttcaagctcactgaccactcactgtacgctatcgctcatggttgccgcgatcttacaaaactaaatatcagtggatgttcagcatttagtgacaatgctctggcctaccttgctggtttctgtagaaaactgaaagttctaaatctctgtggatgtgttagagcagcttctgatactgcacttcaggctattggacactactgcaatcagttgcagtctttgaaccttggatggtgtgacaaagttggtgatgttggagtgatgagtctagcgtatggttgtcctgatctaaggacagttgatttgtgtggctgtgtctatataacagatgatagtgtgattgctttggcaaacggatgccctcacctgaggtcccttggactgtacttctgcaagaacataacagacaatgcaatgtattcattggcacaaagcaaagtgaagaacaggatgtggggatctgtgaaaggaggaaatgatgaagatggactaaggacattgaacatcagccagtgcacttcactcactccttcagctgttcaggcggtttgtgactcatctcctgctctccacacatgctcagggaggcattcactcatcatgagtggctgtctcaatttgaccgaagtccattgtgcgtgtgctggtcatgcacaccgtgcaatgaacgctttcccccatcctgctcattaa

SEQ ID NO: 46 - protein, 368 aa, Medicago truncatula
MVGKDNLKTEDLNLFFENLMMVAGGEKGGNMKVGVITEWKDIPVELLMQILSLVDDQTVIRVSGVCRGWRDSIYFGLARLSLSWCNKNMNNLVLSLVPKFAKLQTLILRQDKPQLDDNVVGTIANFCHDLQILDLSKSFKLTDHSLYAIAHGCRDLTKLNISGCSAFSDNALAYLAGFCRKLKVLNLCGCVRAASDTALQAIGHYCNQLQSLNLGWCDKVGDVGVMSLAYGCPDLRTVDLCGCVYITDDSVIALANGCPHLRSLGLYFCKNITDNAMYSLAQSKVKNRMWGSVKGGNDEDGLRTLNISQCTSLTPSAVQAVCDSSPALHTCSGRHSLIMSGCLNLTEVHCACAGHAHRAMNAFPHPAH

SEQ ID NO: 47 - DNA, 1116 bp, Glycine max
atggttggcaaggagagtttgagaactgaggtcttgaacttgagctttgagaagcttatgatggttgaatgtggtggaaatagtgggaaaggagtgaacattaaagctggtgtgatcacggagtggaaggatattccagttgagcttttgatgcaaattttgtcacttgtggatgatcaaacggttatcatagcttctgaagtgtgtcgtgggtggagagaggcaatttgctttggccttactcggttatcactctcatggtgtagcaagaacatgaataacttggtcctatccctttctcctaaattcacaaaactgcagactttaatccttcgtcaagacaagcctcaactagaggacaatgctgttgaaactatagcaaatttctgtcatgacctccaaatcttggacctcagcaaaagtttcaagcttactgatcgctcattgtatgccgtagcccttggttgtcgggatcttacaaaactgaacatcagtggttgttcagcctttagtgacaatgctctggcttacctggccagtttttgtagaaagctgaaagttttgaatctctgtggatgtgttaaagctgcatctgacactgcgttacaggcaattgggcattactgcaatcagttacagtttttgaaccttgggtggtgtgaaaatgtcagtgatgttggagtgatgagtttaacatatggctgccctgatcttagaacacttgacttatgtggttgtgtccttataacagatgacagtgtaattgtcttggcaaacagatgtcctcatctgaggtcccttgggctgtactactgccaaagcataacagacaaggcaatgtactctttggcacaaagcaaattgaacaacagggtgtgggggtctgtgaaaggtggtggaaatgatgatgatggactaagaactctgaacattagccaatgcacagcactcaccccttctgctgtgcaggctgtgtgtgactcatgcccttccctccacacctgttctggaagacactcactcatcatgagtggttgtctgaacctgacttctgtgcattgtgtttgtgctggccaagcacaccgtgctatcactctccctcatgcagctcattga

SEQ ID NO: 48 - protein, 371 aa, Glycine max
MVGKESLRTEVLNLSFEKLMMVECGGNSGKGVNIKAGVITEWKDIPVELLMQILSLVDDQTVIIASEVCRGWREAICFGLTRLSLSWCSKNMNNLVLSLSPKFTKLQTLILRQDKPQLEDNAVETIANFCHDLQILDLSKSFKLTDRSLYAVALGCRDLTKLNISGCSAFSDNALAYLASFCRKLKVLNLCGCVKAASDTALQAIGHYCNQLQFLNLGWCENVSDVGVMSLTYGCPDLRTLDLCGCVLITDDSVIVLANRCPHLRSLGLYYCQSITDKAMYSLAQSKLNNRVWGSVKGGGNDDDGLRTLNISQCTALTPSAVQAVCDSCPSLHTCSGRHSLIMSGCLNLTSVHCVCAGQAHRAITLPHAAH

SEQ ID NO: 49 - DNA, 990 bp, Glycine max
atgaattcaaacgtgcactcaatttattacgtgcctaagctagcagactcgagttatatgacaaatggatatttggagactgccatcgattggttacctggcatcaaagaaattttgttgagagatttcccaagcttcttcagaaccatagacccacatgatattatgctgcaagttttgcaggaagagtgtgggagagctaagcatgcttctgcaatcattttgaacacatttgaggctttagagcatgatgttttggaggcactatcgtccatgttgcctcctgtgtaccccattggtcccttaactttactcttgaatcatgtcacggatgaggacttgaaaacaattgggtcgaacctttggaaggaagatagagagtgtctaaaatggcttgacaccaatgaacccaagtcagtaatttatgtgaactttgggagcatcacagtcatgactaatcatcaattgattgagtttgcttggggacttgctaacagtggcaaaacctttctgtgggtcattaggcctgatcttgtggacgaaaacacgattcttccttacgagtttgtgttggagactaaagatagaggtcaattgtcaggttggtgtccccaagaggaagtgctagcacaccctgcaattggagggttcttgacacacagtggttggaattcaacaattgagagtttgtgcaatggagtgccaatgatatgttggcctttctttgcggagcaaccgactaattgtcgattctgttgcaaggaatggggtgttgggatgcagatagaaggtgatgttacgagggatagagttgagaggcttgtgagggagttgatggaggggcagaaggggaaagagttgaccatgaaagccttggaatggaagaaactggcagaagatgccaccattctcaaagagggctcttcatttcttaattatgataatatggttcgtcaagttcttttgagtgacaacttgaaatcaacttaa

SEQ ID NO: 50 - protein, 329 aa, Glycine max
MNSNVHSIYYVPKLADSSYMTNGYLETAIDWLPGIKEILLRDFPSFFRTIDPHDIMLQVLQEECGRAKHASAIILNTFEALEHDVLEALSSMLPPVYPIGPLTLLLNHVTDEDLKTIGSNLWKEDRECLKWLDTNEPKSVIYVNFGSITVMTNHQLIEFAWGLANSGKTFLWVIRPDLVDENTILPYEFVLETKDRGQLSGWCPQEEVLAHPAIGGFLTHSGWNSTIESLCNGVPMICWPFFAEQPTNCRFCCKEWGVGMQIEGDVTRDRVERLVRELMEGQKGKELTMKALEWKKLAEDATILKEGSSFLNYDNMVRQVLLSDNLKST

SEQ ID NO: 51 - DNA, 1419 bp, Glycine max
atggattcagttgcattaaatggcaaaagcaatgataagcctcttcacgttgccatgctcccatggctggccatgggtcacatatacccttacttcgaagtcgccaagattcttgctcaaaagggtcactttgttaccttcataaacagccccaaaaacattgaccgcatgcccaaaaccccgaaacacttagaaccattcatcaaactcgtgaagctacccttgccaaaaatagagcatctcccagaaggcgcagagagcacaatggacattccgtccaaaaaaaactgtttcctcaagaaagcttacgagggcctacaatacgccgtttccaagttgctgaaaacgtcaaaccctgactgggttttgtatgacttcgcagctgcttgggtcataccaatagccaagagctacaacattccttgtgctcactacaacattaccccagctttcaacaaagtctttttcgatccaccaaaggacaaaatgaaggattactcgctcgcaagcatatgtggtcctcccacgtggcttcctttcaccacaacgatccatatcaggccttacgagtttttgagagcatacgaaggtaccaaagacgaggagacaggtgaaagggcttcttttgatctcaacaaggcatattcaagctgtgacctttttcttctaagaacctccagagagcttgaaggagactggttggattatcttgctggtaattacaaggttcctgtggttccagttgggttgcttccaccatccatgcagataagggacgttgaagaggaagacaataaccctgattgggtcagaatcaaggattggttagacacacaagagtcgtcatccgtggtttatattgggtttgggagcgagttgaagctgagtcaggaggacctcactgagttggcacatggcattgagctttcaaatttgcctttcttttgggcattgaagaacctgaaagaaggtgtgcttgagttgccagaagggtttgaagaaagaaccaaggaacgtggcattgtttggaagacttgggcaccccagcttaagatcttggctcatggagcaattggagggtgcatgagtcactgtggctctggctctgtgattgagaaggttcattttgggcacgtgcttgtgactctgccttatttgcttgaccaatgtctgttttcgagggtgctggaggaaaagcaagtggctgttgaggttccaaggagcgagaaagatgggtcttttactagggtcgatgtggctaagacattgagatttgcgattgtggatgaggaagggagtgctctaagagagaatgccaaggagatgggcaaggttttcagttccgaagaacttcataataagtatattcaagatttcattgatgcgcttcagaagtataggattccttccgctagctaa

SEQ ID NO: 52 - protein, 472 aa, Glycine max
MDSVALNGKSNDKPLHVAMLPWLAMGHIYPYFEVAKILAQKGHFVTFINSPKNIDRMPKTPKHLEPFIKLVKLPLPKIEHLPEGAESTMDIPSKKNCFLKKAYEGLQYAVSKLLKTSNPDWVLYDFAAAWVIPIAKSYNIPCAHYNITPAFNKVFFDPPKDKMKDYSLASICGPPTWLPFTTTIHIRPYEFLRAYEGTKDEETGERASFDLNKAYSSCDLFLLRTSRELEGDWLDYLAGNYKVPVVPVGLLPPSMQIRDVEEEDNNPDWVRIKDWLDTQESSSVVYIGFGSELKLSQEDLTELAHGIELSNLPFFWALKNLKEGVLELPEGFEERTKERGIVWKTWAPQLKILAHGAIGGCMSHCGSGSVIEKVHFGHVLVTLPYLLDQCLFSRVLEEKQVAVEVPRSEKDGSFTRVDVAKTLRFAIVDEEGSALRENAKEMGKVFSSEELHNKYIQDFIDALQKYRIPSAS

SEQ ID NO: 53 - DNA, 1395 bp, Glycine max
atgcctctacacattgcaatgctcccgtggctcgcagtgggtcatgtgaacccttatttggagctgtcaaagattcttgctcaaaagggacactttgttacctttataagcactccaaaaaacattgatggcatgcccaaaatcccggaaaccttacagccatccatcaaattggtgaggctacccttgccacacacagatcatcatcatcatctcccagaagatgctgagagcacaatggacattccctcaaacaaaagctattacctcaagctggcttacgaggctctccaaggccctgtatcggagttgctcaaaacttcaaagcccgattgggttttttacgatttcgcaaccgagtggttaccaccaatagccaagagcctcaacattccctgtgcacactacaacttaaccgcagcgtggaacaaagtgttcattgatccacccaaggattatcaactgaacaactcaattacgcttcaagacatgtgtctccctcccacgtggcttcctttcaccacaaccgttcatctcaggcctcacgaaatcagaagagcaacaagttctatcaaagattcggacacaggacgcatggcaaattttgatctcagaaaggcatattccagctgcgacatgtttcttctcagaacatgcagagaactcgaaggagaatggttggattatcttgctcacaagtacaaggttcctgtggttccggtagggttggttccaccatccattcagataagggatgttgaagaggaagacaataaccctgactgggtcaaaattaaggattggttggacaaacaagagtcatcatccgtggtttatatagggtttgggagcgagttgaggctgagtcagcaggacgtcaccgagttggctcatggtattgagctttctgggttgcgtttcttttgggctttgaggaacctgcagaaagaggatttgccacatgggtttgaggagagaaccaaggaacgtggcattgtttggaagagttgggcaccccagattaagatcttaggccatgcagctattggaggatgcatcactcactgtggtaccaattctctcgttgagatgcttaattttgggcatgtgcttgtgactcttccctatttgctagaccaagctttgttttcgagggtgctagaggaaaagaaagtgggtattgaggtgccaaggagcgagaaagatgggtcctttactcgagacgatgtggccaagacattaaagttggcaatagtagatgaggaagggagtgattataggaagaatgccaaagaaatgggcaaggttttcagttccacggatcttcatagtcgatacattgacgactgcattgttgctcttcaaaagtacaagactcctaattccaactgttaa

SEQ ID NO: 54 - protein, 464 aa, Glycine max
MPLHIAMLPWLAVGHVNPYLELSKILAQKGHFVTFISTPKNIDGMPKIPETLQPSIKLVRLPLPHTDHHHHLPEDAESTMDIPSNKSYYLKLAYEALQGPVSELLKTSKPDWVFYDFATEWLPPIAKSLNIPCAHYNLTAAWNKVFIDPPKDYQLNNSITLQDMCLPPTWLPFTTTVHLRPHEIRRATSSIKDSDTGRMANFDLRKAYSSCDMFLLRTCRELEGEWLDYLAHKYKVPVVPVGLVPPSIQIRDVEEEDNNPDWVKIKDWLDKQESSSVVYIGFGSELRLSQQDVTELAHGIELSGLRFFWALRNLQKEDLPHGFEERTKERGIVWKSWAPQIKILGHAAIGGCITHCGTNSLVEMLNFGHVLVTLPYLLDQALFSRVLEEKKVGIEVPRSEKDGSFTRDDVAKTLKLAIVDEEGSDYRKNAKEMGKVFSSTDLHSRYIDDCIVALQKYKTPNSNC

SEQ ID NO: 55 - DNA, 1431 bp, Medicago truncatula
atgggttccattggcaatggctacaacaacattgatgatgacaagcctcttcatgttgtaatggttccatggctagctatgggacacatatacccttattttgagttggccaagattcttgctcaaaagggtcactatgttaccttcataaacagccctaaaaatattgaccacatgccaaaaacccctaaaattcttcaaccattcataaaattggttaaactatccttaccgcagatagaacaactcccagaaggtgcggagagtactatggacgttccacacaacatgtttggctgccttaagttggcttatgatggtctccaagatgatgttactgagatactcaaaacatcaaaccctgactgggttttctatgattttgctaccgaatggttaccgtcaatagccaaaagtttgaacatcccttgtgctcattataatataataccagcttggaacacatttttcatggatccacctaaggatcaaatcatgatcaagccggatttcaatcctgaagaaatgtgtggcacacaaaactgggttcctttccaaacaaacactcgtttgaaaccttatgagattattagaacaatatcagattttaaagatgattccggaggaatggctcgtttgaatgtcaacaaagtatattcaagttgtgaccttcatctactcagaacatcaagagagcttgaaagtgaatggttggattatatctctgaacaatacaaggctcctgtcgttcttgttgggttgcttccaccatctatgcagataagagacgatgaggaggaagagaaccatcctgattggttgaaaataaaaaaatggctggattcaagagaatcatcttccgttgtttacattggatttgggagcgagttgaggttaacgcaaaaagatctaaccgaattagctcatggaattgagctttctaggttacctttcttttgggctttgaaaaacctaaaaaagggtacacttgagttacctaaaggattcgaagatcgaaccaaagaacgtggaattgtttggaaaacatgggcaccccagcttaagatcttatcccatggagcaattggggggtgtatgagtcattgtggttcaggttcagtcattgagaagctccattttggacatgttcttgttacattgccttatttgctagatcaatgtttgttttcaagagaactagctgaaaagaaagtggccattgaggttccaagaagtgaggaggatgggtcctttactagggactttgtagccctaacattgaggttagcaataatggatgccgaaggaattatttacaggaacaatgcaaaagagatgggcaagattttcagttcaaaagaacttcacaaaaaatacattgaagatttcattgctgctcttcaaaagtatagggttcattctgagaattaa

SEQ ID NO: 56 - protein, 476 aa, Medicago truncatula
MGSIGNGYNNIDDDKPLHVVMVPWLAMGHIYPYFELAKILAQKGHYVTFINSPKNIDHMPKTPKILQPFIKLVKLSLPQIEQLPEGAESTMDVPHNMFGCLKLAYDGLQDDVTEILKTSNPDWVFYDFATEWLPSIAKSLNIPCAHYNIIPAWNTFFMDPPKDQIMIKPDFNPEEMCGTQNWVPFQTNTRLKPYEIIRTISDFKDDSGGMARLNVNKVYSSCDLHLLRTSRELESEWLDYISEQYKAPVVLVGLLPPSMQIRDDEEEENHPDWLKIKKWLDSRESSSVVYIGFGSELRLTQKDLTELAHGIELSRLPFFWALKNLKKGTLELPKGFEDRTKERGIVWKTWAPQLKILSHGAIGGCMSHCGSGSVIEKLHFGHVLVTLPYLLDQCLFSRELAEKKVAIEVPRSEEDGSFTRDFVALTLRLAIMDAEGIIYRNNAKEMGKIFSSKELHKKYIEDFIAALQKYRVHSEN

SEQ ID NO: 57 - DNA, 1434 bp, Lotus japonicus
atgggttcaacttctccaccttcattgaatggcaatggccaagaagataagcctcttcacattgtcatgctcccatggcttgccatgggtcacatatacccttactttgaagtagccaaggttcttgctcaaaagggtcactctgtcaccttcatcaatagtcccaaaaacattgaccgcatgcccaaaactcccaaaagccttgaaccattcatcaacttggtgaggttacccttgccacacattgagcatctcccagaaggagcagagagcaccatggacattcccacaaacaaaggctgtttccttaagttagcctatgaagggcttcaagatgctgttgctgagattcttcaaacttcaaagcctgattgggttctctatgattttgctgctggatggttgccacctatagctaagagcctcaacatcccttgtgctcattacaacataactccagcttggaacaagtgtttctttgatccaccagagcatgtgaagaagtcaaattttagcattgaaaacatttgtggccctcccacttgggttcctttcaaaacaaccattaaactaaggccttatgagttcatgagagcctttgcagctctcaaagatgagtccacaggaaaatcagcttcctttgatctcaagaaagcatattccagctgtgacttgtttcttctcagaacctccagagagcttgaaggggaatggctagattatcttgctgatacctacaaggtgcctgtggttcctgtggggttgcttccaccatccatgcagataagagatgatgatgaggaagagaaaaaccctgactgggttgaaatcaaagcatggttggacacacaagagccttcaactgtggtgtatataggatttgggagtgagctcaagctgagtcagcaagatctcactgagttagctcatggaattaagctttctgggttgcctttcttttgggctttgaagaacttgaaagagggttcacttgagttacctgaggggtttgaggacagaaccaaggaacgtggggttgtttggaaaacatgggcaccccagcttaagatcttggctcatggtgtcattggagggtgcatgagtcactgtggttcaggttctgcaattgagaaggttcattttgggcatgttcttgtgaccctgccttatttgctggatcagtgtctgttttcaagagcattagaagaaaagaaagtggctattgaggtgccaaggaatgaacaagatggatcctttactagggactccgtggcgaagacattgaagttagcaatagtggatgaggaagggagtatctacaggaagaatgccaaagagatgggcaaggttttcagttctgaagatcttcataatcaatacattgaagatttcattgctgctctccaaaagtacagggttcattccaatagttaa

SEQ ID NO: 58 - protein, 477 aa, Lotus japonicus
MGSTSPPSLNGNGQEDKPLHIVMLPWLAMGHIYPYFEVAKVLAQKGHSVTFINSPKNIDRMPKTPKSLEPFINLVRLPLPHIEHLPEGAESTMDIPTNKGCFLKLAYEGLQDAVAEILQTSKPDWVLYDFAAGWLPPIAKSLNIPCAHYNITPAWNKCFFDPPEHVKKSNFSIENICGPPTWVPFKTTIKLRPYEFMRAFAALKDESTGKSASFDLKKAYSSCDLFLLRTSRELEGEWLDYLADTYKVPVVPVGLLPPSMQIRDDDEEEKNPDWVEIKAWLDTQEPSTVVYIGFGSELKLSQQDLTELAHGIKLSGLPFFWALKNLKEGSLELPEGFEDRTKERGVVWKTWAPQLKILAHGVIGGCMSHCGSGSAIEKVHFGHVLVTLPYLLDQCLFSRALEEKKVAIEVPRNEQDGSFTRDSVAKTLKLAIVDEEGSIYRKNAKEMGKVFSSEDLHNQYIEDFIAALQKYRVHSNS

SEQ ID NO: 59 - DNA, 1431 bp, Lotus japonicus
atggattcaactgctccacttccatttagtggcaatggcgaagataaacctcttcatatagtcatgatcccatggctcgccatgggtcacataaccccatactttgcgctggccaaggttcttgctcaaaagggtcacttcatcacattcataaacagcccaaagaacattgatcgcatgcccaaacccccaaaaagccttgaaccattcatcaacttggtgaggttgcccttgccacccattgagcatctcccagaaggtgcagagagcaccatggacatccccactaacaagggatattacctcaagttggcctatgaaggcctccaagatgctgttgctgagatacttcaaacttcaaagcctgattgggttctatatgattttgcagctgactggttgccaccaacagctaagagcctcaacatcccttgtgcccattacaacataaccccagcttggagcaaatgcttctttgatccaccaaaggatcaggtaaagtcatatttcaatcttgaagacatgtgtagcccacccaagtgggttcctttccacacaaccatgagtctaaggccttatgagatcattagagcatttgcagctgtcaaagatgagtctacaggacgatcaattaattatgatcccaacaaagcatattccagcggtgacttgtttcttctcagaagctccagagaacttgaaggagaatggttagattatgttgctgataggtacaagatgcctgtggttcctgttggcttgcttccaccatccttgcaaataagagatgttgaagaggaagacaaacaccccgactgggtgaaaatcaaagcctggttagacacacaagaaccttcatctgtggtgttcataggatttgggagtgagttgaagctgagccagcaagacctcactgagttggcccatggcattgagctttctgggttgcctttcttctgggctttgaagtacctgaaagatggatcacttgagttacctgaggggtttgaggacagaaccaaggatcgtgggattgtttggaaaacatgggcaccccagccgaagatcttagctcatggggtcattgggggatgcatgagtcactcggctgcaggttctgtcattgagatggttaattttgggcacgttcttgtgaccctgccatattatctggaccagtgtctgttttcaagagcattagaagaaaagaaagtgggaatagaggtaccaaggaacgagcaagatggttcctttactagggagtcggtggcgaagacattgaggttagcaatagttgatgaggaagggagtgtctacaggaagaatgccaaagagatgggcaatgttttcagttccaaagttcttcatgatcaatacattgaagattgcattgctgctcttcaaaaatataggcttcattccaatagataa

SEQ ID NO: 60 - protein, 476 aa, Lotus japonicus
MDSTAPLPFSGNGEDKPLHIVMIPWLAMGHITPYFALAKVLAQKGHFITFINSPKNIDRMPKPPKSLEPFINLVRLPLPPIEHLPEGAESTMDIPTNKGYYLKLAYEGLQDAVAEILQTSKPDWVLYDFAADWLPPTAKSLNIPCAHYNITPAWSKCFFDPPKDQVKSYFNLEDMCSPPKWVPFHTTMSLRPYEIIRAFAAVKDESTGRSINYDPNKAYSSGDLFLLRSSRELEGEWLDYVADRYKMPVVPVGLLPPSLQIRDVEEEDKHPDWVKIKAWLDTQEPSSVVFIGFGSELKLSQQDLTELAHGIELSGLPFFWALKYLKDGSLELPEGFEDRTKDRGIVWKTWAPQPKILAHGVIGGCMSHSAAGSVIEMVNFGHVLVTLPYYLDQCLFSRALEEKKVGIEVPRNEQDGSFTRESVAKTLRLAIVDEEGSVYRKNAKEMGNVFSSKVLHDQYIEDCIAALQKYRLHSNR

SEQ ID NO: 61 - DNA, 585 bp, Glycine max
atgtctaatcatcatcacgtagttcagacctcgccaccgaaactattcggtttcccactaacagagcaggaggaattgaattcagatgcagaaagaaaatacgaggttggagaggacagaaaattcagatgccactactgcaaacgagtattcggaaactctcaggccttagggggccaccaaaacgcccacaagaaagagcgtcagagggcgaggcgtttccaaatccacactcatagacgctccattgcagcaccatcttctgcttctgttctcaactcacatgcaataataagatcagtaccttcagttacagttactacgctaccctcctccatttacttacgaggaggccctcctactacttcttctcttctacccatcttccatcctcgtcaacccaataacactaagcacttcccatcacggcccattttgattccttcttccacctaccaccgtactaattgcactccatcctttccttttcagctttatgcctcaccctctgtactgcaatctgctcctaacgtccgtgatttttctgctgagagcagggaagttgatgtccatctcaaattataa

SEQ ID NO: 62 - protein, 194 aa, Glycine max
MSNHHHVVQTSPPKLFGFPLTEQEELNSDAERKYEVGEDRKFRCHYCKRVFGNSQALGGHQNAHKKERQRARRFQIHTHRRSIAAPSSASVLNSHAIIRSVPSVTVTTLPSSIYLRGGPPTTSSLLPIFHPRQPNNTKHFPSRPILIPSSTYHRTNCTPSFPFQLYASPSVLQSAPNVRDFSAESREVDVHLKL

SEQ ID NO: 63 - DNA, 501 bp, Glycine max
atgtctgatgagacctggccaccgaaactatttggtttcccactaacagagcaggaggacagaaaattcagatgccactactgcaaacgagtattcgggaactcgcaggccttaggaggccatcaaaatgcccacaagaaagagcgtcagagggcgaggcgtttccaaatccacactcatagacgcgccattgcagcagcagcatcgatatcttcacatgcaataataacatcagtaccctccatttacttacgaggacaccctcctactcctacttctcttctacccatcttcgatcctcatcgtcaacccaataacactaagcacttcccatcacgccctactttcattccttcttcttccaactaccaccttactaattacactcaatgctttccttttcggctttatgcctcacccactcatgtactgcaatcctctactcctaaggtccctcatttttctgctgagggggaagttgatgtccatctcaaattataa

SEQ ID NO: 64 - protein, 166 aa, Glycine max
MSDETWPPKLFGFPLTEQEDRKFRCHYCKRVFGNSQALGGHQNAHKKERQRARRFQIHTHRRAIAAAASISSHAIITSVPSIYLRGHPPTPTSLLPIFDPHRQPNNTKHFPSRPTFIPSSSNYHLTNYTQCFPFRLYASPTHVLQSSTPKVPHFSAEGEVDVHLKL

SEQ ID NO: 65 - DNA, 1593 bp, Zea mays
atgaaggagtactggacggcgctggcgtcgctgatgggcgcgtttgcgttcctacagggggtggtgcacgcggtattcccggcggagctacgggcggtggtggtgcggctgctgggcaggctcacgcgggctttctccccctactgctacttcgacgtgacggagatggagggcatgagcaccaacgagatctacgacgccgtgcagctctacctcagcagcaccgcggccccggcgtcgggggcgcgcctcagcctgtcgcgccggctcaacgcctcctccttcactttcgggctcgctgccagcgaccgcgtcgtcgacaccttcgcgggggcggccgtcacgtgggagcacgtggtcgcgccgcgccagggccagggcttctcgtggcggccgctccccgaggagaagcgccggttcacgctccgcatccggcgcggcgacagggacaagctgctcccggcgtacctcgaccacatcatcgccgcggctgtcgacatccggcgccgcagccaggaccggatgctttacacgaacgcgcggggcgggagcatggacgcgcgcggcgtgccgtgggaccccgtgccgttcaagcacccgagcacgttcgacacgctggctatggacccggcgcggaaggcggccatcatggccgacctccgggacttcgccgaggggagcgcgttctacgagcgcacggggcgcgcctggaagcgcgggtacctcctgtacggcccgcccggcaccggcaaatcgagcatgatcgcggccatggccaacttcctcggctacgacgtgtacgacctggagctgaccgaggtgagcagcaacgcggagctccggaagctgctgatgaagacgacgtccaagtccatcatcgtgatcgaggacatcgactgctccgtggacctcacgaaccgcgcgggcgcgccgccgaggccgaagccgcgggcgagcatcgacggcgcgatcgagcaggacgggggcgccggcgcggggcggtccatcacgctctccggcctgctcaacttcacggacgggctctggtcgtgctgcggcgcggagcgcatcttcgtgttcaccaccaaccacatcgagaagctggaccctgcgctgctccggtccggccgcatggacatgcacgtcttcatgtcctactgctcgttccaggcgctgaagatcctcctgaggaactacctcggcttccagggcgacgaggagctggaccgcctcagcgacccggcggtcctgcgcgggctcgaggagtgggtggacgccgcggagatcacgccggcggacgtgagcgaggtgctgatcaagaaccgcaggagcggcaaggcggaggcaatgcgggagctcctggacgccctcaaggcccgcgccgagaagcggcgccggggcagcgcctccggcccggccgcggcggggaaggcggccggcggcggcgagaacgaggaggaggaggaggaggaggaggaggaggaggagaagcgggcgctggagagccccaatgaggggaaggagcaggccggcactgacagccgcgggggcggcggacaagacgaggacgcggacggcaagaaacaggtgtga

SEQ ID NO: 66 - protein, 530 aa, Zea mays
MKEYWTALASLMGAFAFLQGVVHAVFPAELRAVVVRLLGRLTRAFSPYCYFDVTEMEGMSTNEIYDAVQLYLSSTAAPASGARLSLSRRLNASSFTFGLAASDRVVDTFAGAAVTWEHVVAPRQGQGFSWRPLPEEKRRFTLRIRRGDRDKLLPAYLDHIIAAAVDIRRRSQDRMLYTNARGGSMDARGVPWDPVPFKHPSTFDTLAMDPARKAAIMADLRDFAEGSAFYERTGRAWKRGYLLYGPPGTGKSSMIAAMANFLGYDVYDLELTEVSSNAELRKLLMKTTSKSIIVIEDIDCSVDLTNRAGAPPRPKPRASIDGAIEQDGGAGAGRSITLSGLLNFTDGLWSCCGAERIFVFTTNHIEKLDPALLRSGRMDMHVFMSYCSFQALKILLRNYLGFQGDEELDRLSDPAVLRGLEEWVDAAEITPADVSEVLIKNRRSGKAEAMRELLDALKARAEKRRRGSASGPAAAGKAAGGGENEEEEEEEEEEEEKRALESPNEGKEQAGTDSRGGGGQDEDADGKKQV

SEQ ID NO: 67 - DNA, 1527 bp, Sorghum bicolor
cgtgctgcgctggcgcggctgctcggcaggctcacgcgcgccttctccccctactgctacttcgacgtgacggagatggaaggcatgagcaccaacgagatctacgacgccgtgcagctgtacctcagcagcaccgccgcgccggcgtccggggcgcgcctcagcctgtcgcgcccgctcaacgcctcctccttcaccttcgggctcgccgccagcgaccgcgtcgtcgacaccttcgccggctgcgccgtcacgtgggagcacgtggtcgcgccgcgccagggccagggcttctcgtggcgcccgctccccgaggagaagcgccgcttcacgctccgcatccgccgcggcgacagggacaagctgctcccggcgtacctcgaccacatcctcgccgcggcggccgacatcaagcgccgcagccaggaccggatgctctacaccaacgcgcggggcggggtcatggactcgcgcggcctgccctgggaccccgtgccgtttaagcacccgagcacgttcgacacgctcgccatggacccggcgcggaaggcggctatcatggccgacctccgggacttcgccgacggcagcgccttctacgagcgcaccggacgcgcatggaagcgcggctacctcctgtatggcccaccgggcaccggcaagtccagcatgatcgcggccatggccaacttcctcggctacgacgtgtacgacctggagctgaccgaggtgagcagcaacgcggagctccgcaagctgctgatgaagacgacgtccaagtccatcatcgtgatcgaggacatcgactgctccgtcgacctcaccaaccgcgcggccgcgccgccgaagccgaagccgaatccgcggccgagcatcaccgtcgacggcgccatggtcaaccaggacgggggcgccggcggggcgggccagtccatcacgctctccggcctgctcaacttcacggacggcctgtggtcgtgctgcggcgcggagcgcatcttcgtgttcaccaccaaccacatcgagaagctggacccggcgctgctccggtcgggccgcatggacatgcacgtcttcatgtcctactgctcgttcccggcgctcaagatcctgctcaagaactacctcggcttccaggacgacgaggagctggaccgcctcagcgacagcgacgccatgcgcgggctggaggagtgggtggacgccgcggagatcacgccggcggacgtgagcgaggtgctgatcaagaaccgtaggagcggcaagacggaggcaatgcaggggctgctggacgaattcagggcccgcgccgagacgcggggccggggcttcagcgacggagctgccgccgcggcggggaacgaggccgccggcggcgacggcgagaacgaggaggaagaggaggaggaggagaaacgggcgctggagagccccaaggaggccgggaaggagcaggacgtgggcggcattgacagctgcgcggacggacaggatgaggaagcggaggagggaaagaaacaggtgtga

SEQ ID NO: 68 - protein, 537 aa, Sorghum bicolor
MEYWAALASLMGAFAFLQGVVHAMFPAELRAALARLLGRLTRAFSPYCYFDVTEMEGMSTNEIYDAVQLYLSSTAAPASGARLSLSRPLNASSFTFGLAASDRVVDTFAGCAVTWEHVVAPRQGQGFSWRPLPEEKRRFTLRIRRGDRDKLLPAYLDHILAAAADIKRRSQDRMLYTNARGGVMDSRGLPWDPVPFKHPSTFDTLAMDPARKAAIMADLRDFADGSAFYERTGRAWKRGYLLYGPPGTGKSSMIAAMANFLGYDVYDLELTEVSSNAELRKLLMKTTSKSIIVIEDIDCSVDLTNRAAAPPKPKPNPRPSITVDGAMVNQDGGAGGAGQSITLSGLLNFTDGLWSCCGAERIFVFTTNHIEKLDPALLRSGRMDMHVFMSYCSFPALKILLKNYLGFQDDEELDRLSDSDAMRGLEEWVDAAEITPADVSEVLIKNRRSGKTEAMQGLLDEFRARAETRGRGFSDGAAAAAGNEAAGGDGENEEEEEEEEKRALESPKEAGKEQDVGGIDSCADGQDEEAEEGKKQV

SEQ ID NO: 69 - DNA, 1752 bp, Glycine max
atgaacttcagaagcggctcctcggatgacaaggacgccgacaacagcagcaccggcgaggcctatttgagcggcgtttcggacgtcatcacgccgctgaggcgcctctcggagcagctcgggtcaatattagatggcgggggggttgactttttctccgacgccaagatcgtggccggagacggccgggaagtggcggtgaaccgggccattctggcggccaggagcggcttcttcaagcatgtcttcgccggtggtggcggctgcgtgctccggctgaaggaagtggccaaggactacaacgtggggttggaggcgcttgggattgttttggcttatttgtatagtggaagagtgaagcctttgcctcaaggtggtgtttgtgtttgtgtggatgatgtgtgttcacattttgggtgtaggcctgctattgatttcttgcttcagcttctctatgcatcctccacttttcagctcaatgaagatcataacatcttgataatcctattcgtaccagataagattattggacatctactagacattcttgaaaaggtggcaatagatgatattttggtggttctatcggtcgcaaatatatgtggcatagtggccgaaagactactagcaaggtgcactgagatgatacttaaatctgatgctgatatcaccactcttgaaaaggcactgcctcaacatttggtgaagcaaatcacagataaacgaatagaactggacctgtacatgcccgaaaacttcaattttcctgacaaacatgtcaacagaatacacagggcactggattcggacgatgtggaactggtaagattgcttttgaaagagggacacacaactcttgatgatgcttatgcactccactatgctgttgcatactgtgatgtgaagaccaccactgagcttcttgatcttggtcttgctgatgtgaaccacaaaaactataggggctatagtgtgctgcatgttgctgcaatgaggaaggagcctaagattatagtttctctcttgactaagggggcacagccttctgatctcactttggatggaagaaaagcccttcagatttcgaaacgtctcaccaaagctgtggattattataagtccacagaggaaggaaaagtttcatgcagtgacaggttgtgcatagagatattggagcaagctgagagaagggagcctttacttggagaggcatctctttctcttgctatggcaggggatgatttgcgcatgaaactgttgtacctagaaaatagagttggactggcgaaagtgttgtttcccatggaagcaaaggttattatggatatttctcaaattgatggcacatctgagtttccatcaacagacatgtactgtccaaatattagtgatcaccagaggacaactgtggacttaaacgatgcacctttcagaatgaaggaagaacatctagttaggttgagagcactgtctagaactgtggagttggggaaacgcttcttccctcgatgctcagaagtgttgaacaagatcatggatgctgatgacctaactcagcttacatgtatgggtgatgacagtcccgaagatcgactaaggaagcgaagaaggtatgtggagctccaagaagttctgaataaggtcttcaatgaggacaaggaggagtttgataggtctgccatgtcatcatcatcctcttctacatcaattggagtagtgagacctaatgccaatctagccatgaagaactag

SEQ ID NO: 70 - protein, 583 aa, Glycine max
MNFRSGSSDDKDADNSSTGEAYLSGVSDVITPLRRLSEQLGSILDGGGVDFFSDAKIVAGDGREVAVNRAILAARSGFFKHVFAGGGGCVLRLKEVAKDYNVGLEALGIVLAYLYSGRVKPLPQGGVCVCVDDVCSHFGCRPAIDFLLQLLYASSTFQLNEDHNILIILFVPDKIIGHLLDILEKVAIDDILVVLSVANICGIVAERLLARCTEMILKSDADITTLEKALPQHLVKQITDKRIELDLYMPENFNFPDKHVNRIHRALDSDDVELVRLLLKEGHTTLDDAYALHYAVAYCDVKTTTELLDLGLADVNHKNYRGYSVLHVAAMRKEPKIIVSLLTKGAQPSDLTLDGRKALQISKRLTKAVDYYKSTEEGKVSCSDRLCIEILEQAERREPLLGEASLSLAMAGDDLRMKLLYLENRVGLAKVLFPMEAKVIMDISQIDGTSEFPSTDMYCPNISDHQRTTVDLNDAPFRMKEEHLVRLRALSRTVELGKRFFPRCSEVLNKIMDADDLTQLTCMGDDSPEDRLRKRRRYVELQEVLNKVFNEDKEEFDRSAMSSSSSSTSIGVVRPNANLAMKN

SEQ ID NO: 71 - DNA, 1752 bp, Medicago truncatula
atgtacttgagaagtggattctctgaatatgacaacgaaatcgacaacaatactagcagtgagttatgctgcacaacagcaccaaattcaaccattactgcttcacaagatgtcatcacattcacacaaccactcaaccgtctctcagacaatctagcttcaatactagatgatacagggtttgacttcttttccgacgcaaagatcatcgccaaagatggtcgagaagtttccgtgcaccgggccattttatcggcgaggagtagtttctttaaggatgtgtttaagggaaagaaagaaacgacgctgcaactgaaggaagttgctaaggattatgatgttgggtttgacgcgcttaacgttgttctgcgttatttgtatagtgagagagttgaggatcatcatttgtctgctaaagatgtttgtgtttgtgtggatgatgattgtttacactttggttgttggccagttgttgatttcatgcttcaacttctctatgcttccttcacctttcaaatttctgaattgcttgctctttaccaagatcatctactagacattcttgacaaaatggcgatagatgacatgttggtggttctttctatagcaaacatttgtggtaaaacagccgataaactactaaaaagatgtacagatataatagttgaatccaatgttgatataaccactcttgaaaaatcactgcctcaatctatagtgaaactagtaacatacaaacgcaaacaactaggtttagacatgtatgaaaccgtaaatcttctagacaaacatgtcaccagaatacatagagctttagattcagatgatgtagaattagtaaggttgcttttgaaagaaggacacactactctagatgaggctcatgcacttcactatgctgttgcatattgtgatgtcaaaaccactaccgagcttcttgatcttggacttgctgatgtaaatcataaaaaccttaggggatacagcgtgctacatgttgctgctaagagaaaagaaccgaaaattattgtatctttgttaacgaagggagctcaaccttctgaactcactatggatggcagaaaagctcttcagatttcaaaacgttgtacgaaagctgtggattattataaatctactgaggaaggaaaagtttcttcgaatgatcgattgtgcatagagatattggagcaagctgagagaagggagcctttacatggggaggcatctctttctcttgctaaggcaggggatgatttgcgtatgaaattgttgtacctggaaaaccgagttggattggcaaaacttttgtttcccatggaagcaaaggttgtgatggatatcactccaattgatggcacatctgaatttactccaaatttgggtggttaccagaggacaactatggacttgaatgaggcaccttttaaaataaaagaggagcatctaattcggatgaaggcattgtctagagcagtggagttagggaaacggttcttccctcgttgctcggaagtgttgaacaagatcatggatgccgatgacctatcgcagcttgcatgcatgggtcatgacagtcctgaagatcgacaagtgaagcgcagacgatacgcggagctacaagaagttcttaataaggtcttccatgaggacaaagaggagtttgataaatctggcatgtcttcatcatcctcatccacgtcaataggaatgccaagggctaataatagtatgatagccatgaatcattga

SEQ ID NO: 72 - protein, 583 aa, Medicago truncatula
MYLRSGFSEYDNEIDNNTSSELCCTTAPNSTITASQDVITFTQPLNRLSDNLASILDDTGFDFFSDAKIIAKDGREVSVHRAILSARSSFFKDVFKGKKETTLQLKEVAKDYDVGFDALNVVLRYLYSERVEDHHLSAKDVCVCVDDDCLHFGCWPVVDFMLQLLYASFTFQISELLALYQDHLLDILDKMAIDDMLVVLSIANICGKTADKLLKRCTDIIVESNVDITTLEKSLPQSIVKLVTYKRKQLGLDMYETVNLLDKHVTRIHRALDSDDVELVRLLLKEGHTTLDEAHALHYAVAYCDVKTTTELLDLGLADVNHKNLRGYSVLHVAAKRKEPKIIVSLLTKGAQPSELTMDGRKALQISKRCTKAVDYYKSTEEGKVSSNDRLCIEILEQAERREPLHGEASLSLAKAGDDLRMKLLYLENRVGLAKLLFPMEAKVVMDITPIDGTSEFTPNLGGYQRTTMDLNEAPFKIKEEHLIRMKALSRAVELGKRFFPRCSEVLNKIMDADDLSQLACMGHDSPEDRQVKRRRYAELQEVLNKVFHEDKEEFDKSGMSSSSSSTSIGMPRANNSMIAMNH

SEQ ID NO: 73 - DNA, 1755 bp, Vitis vinifera
atggactacagagctgctctctccgatgacaacgacttcgacggcagtagcagcatctgctgcatcgcagccaccaccgaatccctttcctcagaggtttctccgccggacatctcggctctcaggcgcctttcggagaatctcgagtcggtgttcgagtcccccgagtttgacttcttcacagacgccaggatcgtggtcgccggaggccgggaggtgccggtgcaccgggccattcttgcggcgaggagcgtgtttttcaaggccgtgttggcgggggcgaggaaggagaaggaggcgaagttcgagctgaaggatttggcgaaggaattcgatgttgggtacgactcgctcgtggcggttttggggtatttgtatagtgggagagttggggcgttgccaaaaggcgtttgtgcgtgcgttgatgatgattgcccccactccgcttgtaggcccgctgtggatttcatggtggaggtgctctatgcttctttcgcttttcagatctctgaattggtcggcctttatcagagacgcctgatggacattcttgacaaggttgcttcagatgacattttagtgattctatcagttgctaatctgtgtggtaaagcagccgatagattgttagcgaggtgtattgacattatcatcaaatctgatgtggatgtcgtgactctggagagggccttgccccaagaaatggtgaaacagattgtggattcgcgattggagcttggctttgaggagcctgaaagcaccaattttcctgacaaacatgtgaagaggatacaccgggctctggattcagatgatgttgaattggtgaggatgctactgaaagagggacatactactctagatgatgcatatgcccttcactatgctgtggcatttggtgatgcaaagaccacaaccgagcttcttgatcttggacttgctgatgttaaccataaaaatcataggggctatactgtgctacacattgctgcaatgcggaaagagcccaagattatagtatcccttcttactaagggagctcggccaactgatatcacgccagacggtagaaatgcactgcaaatagcaaagaggctcaccagggctgtggattaccataagtctaccgaggaaggaaaaccttctcccaaggatcagttgtgcgtagaggtattggagcaagcggaaagacgagatccattgataggggaggcttcgttttctcttgctatagcaggcgatgatctgcgaatgaagctgttgtacctcgaaaatagagttggactagcaaaacttctattccccatggaagctaaagtggcaatggatattgcacaagtggatggcacttctgagtttacactcactgcaatcaggcctaggaatttggctgatgcccagaggacaactgtggacttaaacgaggcgcctttcagaattaaagaggagcacctaaataggctaagagcactgtccaaaactgtggaccttgggaaacgctttttccctagatgctcagaagtactcaacaagatcatggatgccgatgacttatcagatttggcatacctgggcaacggcactacagaggagcgacttctgaagaagaggaggtacaaggaactccaggaccaactgtgcaaggcattcaatgaagacaaagaggagaatgataagtctaggatatcatcctcatcatcatccacatctctagggtttggaaggaataacagtagactctcttgcaagaagtaa

SEQ ID NO: 74 - protein, 584 aa, Vitis vinifera
MDYRAALSDDNDFDGSSSICCIAATTESLSSEVSPPDISALRRLSENLESVFESPEFDFFTDARIVVAGGREVPVHRAILAARSVFFKAVLAGARKEKEAKFELKDLAKEFDVGYDSLVAVLGYLYSGRVGALPKGVCACVDDDCPHSACRPAVDFMVEVLYASFAFQISELVGLYQRRLMDILDKVASDDILVILSVANLCGKAADRLLARCIDIIIKSDVDVVTLERALPQEMVKQIVDSRLELGFEEPESTNFPDKHVKRIHRALDSDDVELVRMLLKEGHTTLDDAYALHYAVAFGDAKTTTELLDLGLADVNHKNHRGYTVLHIAAMRKEPKIIVSLLTKGARPTDITPDGRNALQIAKRLTRAVDYHKSTEEGKPSPKDQLCVEVLEQAERRDPLIGEASFSLAIAGDDLRMKLLYLENRVGLAKLLFPMEAKVAMDIAQVDGTSEFTLTAIRPRNLADAQRTTVDLNEAPFRIKEEHLNRLRALSKTVDLGKRFFPRCSEVLNKIMDADDLSDLAYLGNGTTEERLLKKRRYKELQDQLCKAFNEDKEENDKSRISSSSSSTSLGFGRNNSRLSCKK

SEQ ID NO: 75 - DNA, 1770 bp, Populus trichocarpa
atggataataggattggtttctctgatgacaacgagatcgacaatggaagtagcacttgctgcatagaaactccctcaacttcaaagccttttacaaacccagaaattgtagctcttcaacaactatcaggaaaccttgaagccatctttgactcccaagattttgactactttgctgatgccaaaatcacaagctccaattacaaccgtgaagtcccagttcacagggccattttatctgcgagaagtccttttttcaagagtgttttctcgagtcctgtggctaaagacagaagtggggtagcaaagtttgaattgaaggaattggctaaggactatgatgttggatttgattcacttatgacagtcttgggttatttatattgtgggaaagtgaggccttggcctaaagatgtttgtgcttgtgtggatgatgattgttcccatattgcttgccggcctgcagttgatctcctgactgaggttctttatgcctcttttacattccaggttaatgagttggtggctctttatcagaggcacctattagacattcttgacaaggtttcaactgatgacatcttggtgattctagcagttgcaaacatatgtggtgaagctgccgagagattgctaacaagatgcgttgagattattgtgaaatcaaatgttgatattgttactctggataaagccttgccgcaatacattgtgaagaaaatcatggattctcgtttggaacttggtttgaatgtgcctgagaacagtaacttactagataaacatgtaaagagaatacatcgggctctggactcggatgatgttgagctagttagaatgctactgaaagaggcacatactaatctagatgatgcgcatgcactccactatgctgtttcatactgtgatgcaaagactacaactgagattcttgatcttggattagctgatgttaactgcagaaattcaaggggttacaccgtgctgcatgtcgctgcaatgcggaaagatcctaagattatagtatctctcttaacgaagggagctcggctgtcagatcttactttagatggtaggaaggctctgcagatatcaaaacgacttacaagggccatggattatcataagtcaacagaggaaggaaaagcttctcccaaggagcgcttgtgtatagaaatattggaacaagcagaaaggagggatccattacttggagaggcttctctttctcttgctatggctggtgatgacctgcgaatgaaattgttgtacctcgaaaatagagttggactggcaaaactcctttttcccatggaagcaaaagttgcaatggatattgctcaggttgatggtacttctgagtttccattagctggcatcaggcccagcatcttgtctggggcacaaagaggagctgtggacttgaacgaggcacctttcagaatgcacgaggagcatctaaataggatgagagcactctctagaacagtggaacttgggaaacggtttttcccacgttgctcagacgtactcaacaagatcatggatgctgatgacctatcacagatagcttatttagggaatgaaacttcagaggaacgacttgtgaagagacagagacaccttgaactccaggatgctctaagcaaggcattcaatgaagataaacaagagttcgacaggtctgtcatatcatcttcatcatccaccaagtcaattggcacggcgaggtctaatgggaagcttattgacatgggtggtggtcattag

SEQ ID NO: 76 - protein, 589 aa, Populus trichocarpa
MDNRIGFSDDNEIDNGSSTCCIETPSTSKPFTNPEIVALQQLSGNLEAIFDSQDFDYFADAKITSSNYNREVPVHRAILSARSPFFKSVFSSPVAKDRSGVAKFELKELAKDYDVGFDSLMTVLGYLYCGKVRPWPKDVCACVDDDCSHIACRPAVDLLTEVLYASFTFQVNELVALYQRHLLDILDKVSTDDILVILAVANICGEAAERLLTRCVEIIVKSNVDIVTLDKALPQYIVKKIMDSRLELGLNVPENSNLLDKHVKRIHRALDSDDVELVRMLLKEAHTNLDDAHALHYAVSYCDAKTTTEILDLGLADVNCRNSRGYTVLHVAAMRKDPKIIVSLLTKGARLSDLTLDGRKALQISKRLTRAMDYHKSTEEGKASPKERLCIEILEQAERRDPLLGEASLSLAMAGDDLRMKLLYLENRVGLAKLLFPMEAKVAMDIAQVDGTSEFPLAGIRPSILSGAQRGAVDLNEAPFRMHEEHLNRMRALSRTVELGKRFFPRCSDVLNKIMDADDLSQIAYLGNETSEERLVKRQRHLELQDALSKAFNEDKQEFDRSVISSSSSTKSIGTARSNGKLIDMGGGH

SEQ ID NO: 77 - DNA, 1770 bp, Ricinus communis
atggattataggattggtttctctgatgacaacgaaattgacaatggaagcagcagttgctgtatagaaaccctttcaaatccaaatccaccaattccaaatccagaaatttcagctctccaacagctctcgagaagccttgagtccatcattgaatcattagattttgacttctatgctgatgccaaaattacaatctctgcctctaatagagaggtccctgttcaccgggccattttatcagcgagaagtcctttctttaaggctatgttttcaggttctctgggtaaagagaaaggtgctgtcaagtatgaactgaaggagttgaccaaggattatgatgttgggtttgattctctggtggccgttttgggttatttgtatagtggaaaagtgaggcctttgcctaaaggtgtttgtgtttgtgttgatgaagattgctcccatgttgcttgtaggcctgctgttgatttcatggttgaggtgctatatgcatcttttacttttcaggtgcctgagctagtggctctctatcaaaggcatcttctagacattcttgacaaggttgcaattgatgacatcctggtggttctatctgttgcaaatacgtgtggtaaagctgccgagagattattcacaagatgtatcgagatcattgtgaaatctgatgctgatattgtaactctggataaggccttaccccaacacattgtaaagcaaattacagattctcgctcagagcttggtttggacacacctgaaagcacaggttatcctgataaacatgtaaagagaatacatcgagcgttggactcggatgatgtagaattagttaggatgctgctgaaagaggcacataccaatctggatgatgcacatgcactacattatgctgttgcatattgtgatgcaaagacgacaacggagcttcttgatcttggaattgctgatgtcaactgcagaaactcaaggggctacactgtgcttcatgttgccgcaatgaggaaagagcctaggattattgtaacactcttgacaaagggagctcggccgtcagatctcacttcagatggtcgaaaagcacttcaaatctcgaagcaacttactagagctgcagattactataagtccactgaggaaggaaaagcttctcccaaggaaagattatgcatagagatcttggagcaagcagaaagaagagatccattacatgtagaggcttctctttctcttgctatggctggtgatgacctgcgaatgaaattgttataccttgaaaatagagttggactggcaaaacttctattccctatggaagcgaaagttgcaatggatattgctcaagtggatggtacatatgaatttccactcactaacattgagactaaggctttgtctggtgctcaaaggacaactgtggacttgaacgaggcacctttcaggattcaagaggagcatctgaacaggatgaaagcactgtctagaactgtggaacttggtaaacgctttttccctcggtgttcagaggtactgaaccggatcatggatgcagacgacttatcacagctcgcgtacttaggaaaagatactgtagaagaacgacaccagaagaaacaaagatatatggaactccaggaccttctgagtaaggcatttaatgaggataaacaagagtttgataagtctaacatttcatcctcttcgtcttcgaaatcaatagggatggtgaaatctaatggtaagctccgtaataggaatggaagggcttaa

SEQ ID NO: 78 - protein, 589 aa, Ricinus communis
MDYRIGFSDDNEIDNGSSSCCIETLSNPNPPIPNPEISALQQLSRSLESIIESLDFDFYADAKITISASNREVPVHRAILSARSPFFKAMFSGSLGKEKGAVKYELKELTKDYDVGFDSLVAVLGYLYSGKVRPLPKGVCVCVDEDCSHVACRPAVDFMVEVLYASFTFQVPELVALYQRHLLDILDKVAIDDILVVLSVANTCGKAAERLFTRCIEIIVKSDADIVTLDKALPQHIVKQITDSRSELGLDTPESTGYPDKHVKRIHRALDSDDVELVRMLLKEAHTNLDDAHALHYAVAYCDAKTTTELLDLGIADVNCRNSRGYTVLHVAAMRKEPRIIVTLLTKGARPSDLTSDGRKALQISKQLTRAADYYKSTEEGKASPKERLCIEILEQAERRDPLHVEASLSLAMAGDDLRMKLLYLENRVGLAKLLFPMEAKVAMDIAQVDGTYEFPLTNIETKALSGAQRTTVDLNEAPFRIQEEHLNRMKALSRTVELGKRFFPRCSEVLNRIMDADDLSQLAYLGKDTVEERHQKKQRYMELQDLLSKAFNEDKQEFDKSNISSSSSSKSIGMVKSNGKLRNRNGRA

SEQ ID NO: 79 - DNA, 1776 bp, Gossypium hirsutum
atggatcatagaaatggattttcggatgacaacgaaatcgacaacaacaacagcaccacgtgctgtatcgtccccgccgctgcgacaaccacctccgaaacacttgtttcgtcggaaccgttgaacactccggacgtcgccgctcttcaactcctctccaagaacctcgagtccctttacgaatcgacggattccgattacttttactccgacgcaaaaatcgcgctatcttccggtcgggaggtggccgttcaccgggccattttgttagcgaggagttcggtgtttaagaccgtgttctccgggctgaaagatagtggagctaagtttgagctcaaagagttggctagagactatgagatcggctataattcgcttgttgccgttctggcttacttgtataccggaaaagttaaatcgttgcctaaaggcgtttgcctttgcgtagacgatggttgttcgcacgttggatgtagaccggccgttgatttcattgctgaagttttatatgcagcgttcgtttttcaggtccctgaattaattgctctttatcagagacatctactggacatcatcgactgggtcgccgtaaatgatatcttggtggttctttacattgcaaatatgtgcggcaatgttgccgaaaaattggtgtcaaagtgtgtagaaatcgttgtgaaatccgatgttgatattgtaacactcgacaaagccttgcctcaacccattgtgaaacaaatcattgattcccgtctggaactcagtttagacaagcccgagaacgtaggttttcccgataaacatgtgcggcggattcatcgggcgctggaatcggatgacgtcgagttagttcgaatgctactgaaagagggtcatacgaatttagacgaagcatatgcgcttcactacgctgtggcatactgtgacgcaaagaccacgactgagttgctcgaccttggactcgctgatgttaaccataggaactcgagagggtacacagtgttgcatgttgcggcgatgaggaaagagcctaagattatagtttctcttttaacgaaaggtgcacgaccatccgatctcactatcgatggtagaaaagctcttcaaatatcgaagcggctcacccgggctgccgattactataaatcgaccgaggaaggtaaggcttcgccgaaggacaggttgtgtatagagatactagagcaagctgaaagaagagatccattgcatggtgaagcttctttgtctcttgccatcgctggtgatgatcttcggatgaagttgttgtatctcgaaaatagagttgggctagcgaaacttttattcccaatggaagctaaagttgtgatggatatagctcaagtggatggaacatcggagttcacatttgctaccatcaattccaataaattaaatggtgctcaaacaacagtggacttgaatgaggcacctttcaggattcaagaggagcatttaaatagactcaaagcactttccagaacagtggaactcggtaagcgtttctttccccggtgttccgaagtgttgaacaagatcatggacgcggacgacctatcgcagctagcttgcggagggattgataccgcggaggaacgagtggttaaaaggcagagatacatggaactccaagacgtactaagcaaggcattccatgaagataaagagcagtttgacaggtcagccatctcctcttcatcttcatcaaaatccatagttgtgaccgggcctaaaggtaaagctcactgctacagctaa

SEQ ID NO: 80 - protein, 591 aa, Gossypium hirsutum
MDHRNGFSDDNEIDNNNSTTCCIVPAAATTTSETLVSSEPLNTPDVAALQLLSKNLESLYESTDSDYFYSDAKIALSSGREVAVHRAILLARSSVFKTVFSGLKDSGAKFELKELARDYEIGYNSLVAVLAYLYTGKVKSLPKGVCLCVDDGCSHVGCRPAVDFIAEVLYAAFVFQVPELIALYQRHLLDIIDWVAVNDILVVLYIANMCGNVAEKLVSKCVEIVVKSDVDIVTLDKALPQPIVKQIIDSRLELSLDKPENVGFPDKHVRRIHRALESDDVELVRMLLKEGHTNLDEAYALHYAVAYCDAKTTTELLDLGLADVNHRNSRGYTVLHVAAMRKEPKIIVSLLTKGARPSDLTIDGRKALQISKRLTRAADYYKSTEEGKASPKDRLCIEILEQAERRDPLHGEASLSLAIAGDDLRMKLLYLENRVGLAKLLFPMEAKVVMDIAQVDGTSEFTFATINSNKLNGAQTTVDLNEAPFRIQEEHLNRLKALSRTVELGKRFFPRCSEVLNKIMDADDLSQLACGGIDTAEERVVKRQRYMELQDVLSKAFHEDKEQFDRSAISSSSSSKSIVVTGPKGKAHCYS

SEQ ID NO: 81 - DNA, 1776 bp, Theobroma cacao
atggataacagaaatggcttttcggatgacaacgaaatcgacaacaacagcagcacgtgctgcatcgcggcagcaaccaacagcgaaacgctcgcttcatcagaaccgttgaacactcccgacatagcagctcttcaaattctttccagaaacctcgagtccgttttcgaatcaacggactccgattccttatactccgatgccaaaattgggctttcctcgggccgtgaagtccccgtccaccgcgccattttatcggcgaggagttccgttttcaagaccgtgttttcggggctaaaagatagaggagctaagttcgagctgaaagagttggctagggactatgagattggctatgattctcttgttgcggttctggcttacttgtatagtggaagagtgagatcgttgccgagaggcgtttgcgtttgcgttgacgatgattgctcccacttggcttgcaggccggctgttgatttcgtggctgaagttttatacgcagcttttacttttcaggtctctgaattgatttccctttatcagaggcatcttttagacattatcgacaaggttgaaatggatgatatattggtggttctttatgttgcaaatatgtgcggtaatactgccgaaagactgctggcaaagtgtatagagactcttgtaaaatctgatgttgatattgtaacacttgacaaggccttgccttatcacattgtgaaacaaatcatggattcccgtctggaacttggtttggacaagcctgagaacacaggttttcctgataaacatgtgaagaggatacatcgtgctttggattcggatgatgttgaattagcgagaatgctactgaaagagggtcatactaatttagatgaggcaagtgcacttcactatgccgtggcatactgtgatgcgaagaccacaactgaattacttgaccttggacttgctgatgttaaccgtagaaactcaaggggatacactgtgttgcatgttgcggcaatgaggaaagagcctaagattatagtatctcttttaaccaaaggtgcccggccatctgatctcaccctagatggtaggaaagcttttcagatctcaaagcgactcaccagggctgcagattactatatgtctactgaggaaggaaaggcttctccaaaggaccgtttgtgcgttgagatactggagcaggcagaaagaagagatccattgcttggagaagcttctctttctcttgccatggctggtgatgatctacggatgaaattattgtatcttgaaaatagagttggattggcaaaacttcttttccccatggaagcaaaagttgcgatggacattgctaaagtggatggaacatctgagttcacattagccagcatcaattccaacaaattaaatgatgcccaaagaacaactgtggacttgaatgaggcaccttttagaattcaggaggagcatctaaataggctgaaagcactttctagaacagtggagctcggcaaacgctttttccctcgttgctcagaagtgctgaacaagatcatggacgctgacgacttatctcagctagcatgcggaggaaatgacactccagaggagcgacttgttaaaaagcaaaggtacgtggaactccaagatgtactgagcaaggcattcaatgaggataaagtagagtttgacagatcaaccatctcatcctcttcttcatcaaagtcaataggggtgagcagacctaatggtaagctaactggtagtggtaggggcggttag

SEQ ID NO: 82 - protein, 591 aa, Theobroma cacao
MDNRNGFSDDNEIDNNSSTCCIAAATNSETLASSEPLNTPDIAALQILSRNLESVFESTDSDSLYSDAKIGLSSGREVPVHRAILSARSSVFKTVFSGLKDRGAKFELKELARDYEIGYDSLVAVLAYLYSGRVRSLPRGVCVCVDDDCSHLACRPAVDFVAEVLYAAFTFQVSELISLYQRHLLDIIDKVEMDDILVVLYVANMCGNTAERLLAKCIETLVKSDVDIVTLDKALPYHIVKQIMDSRLELGLDKPENTGFPDKHVKRIHRALDSDDVELARMLLKEGHTNLDEASALHYAVAYCDAKTTTELLDLGLADVNRRNSRGYTVLHVAAMRKEPKIIVSLLTKGARPSDLTLDGRKAFQISKRLTRAADYYMSTEEGKASPKDRLCVEILEQAERRDPLLGEASLSLAMAGDDLRMKLLYLENRVGLAKLLFPMEAKVAMDIAKVDGTSEFTLASINSNKLNDAQRTTVDLNEAPFRIQEEHLNRLKALSRTVELGKRFFPRCSEVLNKIMDADDLSQLACGGNDTPEERLVKKQRYVELQDVLSKAFNEDKVEFDRSTISSSSSSKSIGVSRPNGKLTGSGRGG

SEQ ID NO: 83 - DNA, 1999 bp, Arabidopsis thaliana
gtagtgcccttcatggataccaaaagagaaaatttgatttagtgcatacatataacaatataacgccgcataataatactgtataaaacagtcatgtaacgatatgacagcagtaatacagttccaagagacgttataatcgtatgcaatcatatgcttgcgtagattttccaacagttttgtttcgttgataggaggaactcaacactctagggtagtgattggtagacactattagcacaaaaaatattaattttactctgatgtttaccaaaaaagttaccaatcaaatatttaagagatcgtactcttccacggcgactctaaaaaccaaagatataggttagactcataactactttataaagaaaatgtttaacgataactaccgagatctaataaataaaccttcattttcaagtatattatatttgcttcttttgtttatatatcaaaccaagttctggtttataaaaatattagataaaactcgtctaaataggtaggtgtaaaataaaattttaaatttttatcgataatatttaaaatttgaaaagttaataatgatccacacattttttctaatatttaatttagtaatttttgtattaaataaaatttcaatcatatacattcgatttttctatacattttaactatctatttctgcataataaactgtattttcattttatacgcttcatcttatggatgatatttaaattttaaatagtaattcatacactttttaatatttaatttagtattttcttaaatccaaattttaatcttacaatttaaatatctactttaacataatacaaatacaatttaatttcattgtattaaattcaaatataatttgattataataaaatacaatttaattctaaaaagtccatcttagattttaattttcctttttagttttgaaaattaaaaatttaaatttattagatatatatgttactttttcagttttcctatttatttaagaaaaaaatattttttaacacatgtcaacttgtaaacaatagactgaacacgtcattttatattatgtttagttttgaaaattaaagttaattaaatatttatatttcttttttttagcttttctaattatttttaaaatagtaaatatttttaatacaaatcaatatctgaacaatagatttgatacataacataatcctataaattattaacttggaaaacgatagtttatataataaaattattttcttaagttctctaaccataacaattaaactatattttagcgaagaaaagaagagaataccgagagaacgcaacttgcactaaaagctaccactttggcaaatcactcatttatattattatatactatcacctcaattcaatcgaaacctcaaaataacactaatatatacacaaagaaacaacagaataacaccgaagaatataggtttaggaaaatccagaatttgttgagactaaagagatcaaattttcgatacaaggttttgctcaatttgtattttcataataaaattctttatttcaccatagacttacatgattagtttttcttttaataaaaaaaaacacgcgacatgaaaattatattatctcagtgttgtcgaatttgaatttgaattttgagttaaatactacacatttgttgacaacttattaaactttacaagtctgctacaaatattgtcaaatatttactaattaatggaccaaaatcctctaacttgcaaatttgtatctacatcaacttaaaaattaggaatatgcgacccaaaaaaaaaaaaactaggaataataataaaaaaatggaatgatgtggaggaagctctttactctttgagaggaagtttataaattgaccacacatttagtctattatcatcacatgtattaagacttgacaacttgtctttctcacaccaaacccctctcctctgtttcataacatctgctctttcttttttttcctaagccccta

SEQ ID NO: 84 - DNA, 325 bp, Petroselinum crispum
aattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctc

SEQ ID NO: 85 - DNA, 1052 bp, Artificial sequence
  synthetic
gtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcga

(19) United States
(12) Patent Application Publication
(10) Pub. No.: US 2014/0026256 A1
(43) Pub. Date: Jan. 23, 2014

(54) Nematode-Resistant Transgenic Plants

(71) Applicants: Bonnie McCaig, Durham, NC (US); Aaron Wiig, Durham, NC (US); Steven Hill, Cary, NC (US)

(72) Inventors: Bonnie McCaig, Durham, NC (US); Aaron Wiig, Durham, NC (US); Steven Hill, Cary, NC (US)

(73) Assignee: BASF Plant Science Company GmbH

(21) Appl. No.: 13/991,626

(22) PCT Filed: Dec. 19, 2011
(86) PCT No.: PCT/EP2011/073158

§ 371 Date: Aug. 12, 2013

Related U.S. Application Data
(60) Provisional application No. 61/424,866, filed on Dec. 20, 2010.

(51) Int. Cl. C12N 15/82

(52) U.S. Cl. CPC C12N 15/8285
USPC 800/279; 800/298; 435/320.1

(57) ABSTRACT

The invention provides nematode-resistant transgenic plants and seed produced by expression of polynucleotides encoding certain plant polypeptides. The invention also provides methods of producing soybean cyst nematode-resistant transgenic plants in which those plant polynucleotides are expressed and expression vectors for use in such methods.

### FIELD OF THE INVENTION

The invention relates to enhancement of agricultural productivity through use of nematode-resistant transgenic plants and seeds, and methods of making such plants and seeds.

### BACKGROUND OF THE INVENTION

Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore parasitic on a large number of crop species. The cyst- and lesion-forming nematode species have a more limited host range, but still cause considerable losses in susceptible crops.

Parasitic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. Nematode infestation, however, can cause significant yield losses without any obvious above-ground disease symptoms. The primary causes of yield reduction are due to underground root damage. Roots infected by SCN are dwarfed or stunted. Nematode infestation also can decrease the number of nitrogen-fixing nodules on the roots, and may make the roots more susceptible to attacks by other soil-borne plant nematodes.

The nematode life cycle has three major stages: egg, juvenile, and adult. The life cycle varies between species of nematodes. The life cycle of SCN is similar to the life cycles of other plant parasitic nematodes. The SCN life cycle can usually be completed in 24 to 30 days under optimum conditions, whereas other species can take as long as a year, or longer, to complete the life cycle. When temperature and moisture levels become favorable in the spring, juveniles hatch from eggs in the soil. Only nematodes in the juvenile developmental stage are capable of infecting soybean roots.

After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN, migrate out of the root into the soil and fertilize the adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years.

A nematode can move through the soil only a few inches per year on its own power. However, nematode infestation can spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. For example, U.S. Pat. Nos. 5,589,622 and 5,824,876 are directed to the identification of plant genes expressed specifically in or adjacent to the feeding site of the plant after attachment by the nematode. A number of approaches involve transformation of plants with double-stranded RNA capable of inhibiting essential nematode genes. Other agricultural biotechnology approaches propose to over-express genes that encode proteins that are toxic to nematodes.

To date, no genetically modified plant comprising a transgene capable of conferring nematode resistance has been deregulated in any country. Accordingly, a need continues to exist to identify safe and effective compositions and methods for controlling plant parasitic nematodes using agricultural biotechnology.

### SUMMARY OF THE INVENTION

The present inventors have discovered that transgenic overexpression of certain plant polynucleotides can render plants resistant to parasitic nematodes. Accordingly, the present invention provides transgenic plants and seeds, and methods to overcome, or at least alleviate, nematode infestation of valuable agricultural crops.

In one embodiment, the invention provides a nematode-resistant transgenic plant transformed with an expression vector comprising an isolated polynucleotide encoding a polypeptide selected from the group consisting of a) a transferase comprising amino acids 1 to 448 of SEQ ID NO:2; b) a senescence related oxidoreductase having at least 69% global sequence identity to SEQ ID NO:4; c) a histidine phosphotransfer kinase/transferase having at least 73% global sequence identity to SEQ ID NO:16; d) an AP2/EREBP polypeptide comprising a first conserved domain which is at least 94% identical to a domain comprising amino acids 138 to 253 of SEQ ID NO:28 and a second conserved domain which is 100% identical to a DNA binding motif comprising amino acids 252 to 303 of SEQ ID NO:28; e) a basic helix loop helix polypeptide comprising amino acids 1 to 481 of SEQ ID NO:38; f) an auxin inducible polypeptide comprising amino acids 1 to 172 of SEQ ID NO:40; g) an F box and LRR polypeptide having at least 85% global sequence identity to SEQ ID NO:42; h) a glucosyl transferase comprising amino acids 1 to 329 of SEQ ID NO:50; i) a glucosyl transferase having at least 72% global sequence identity to SEQ ID NO:52; j) a zinc finger polypeptide selected from the group consisting of SEQ ID NO:62 and SEQ ID NO:64; k) an AAA ATPase selected from the group consisting of SEQ ID NO:66 and SEQ ID NO:68; and l) a polypeptide comprising a BTB/POZ domain and an ankyrin repeat domain and having at least 67% global sequence identity to SEQ ID NO:70.

Another embodiment of the invention provides a seed produced by the transgenic plant described above. The seed is true breeding for a transgene comprising at least one polynucleotide encoding a polypeptide selected from the group consisting of a) a transferase comprising amino acids 1 to 448 of SEQ ID NO:2; b) a senescence related oxidoreductase having at least 69% global sequence identity to SEQ ID NO:4; c) a histidine phosphotransfer kinase/transferase having at least 73% global sequence identity to SEQ ID NO:16; d) an AP2/EREBP polypeptide comprising a first conserved domain which is at least 94% identical to a domain comprising amino acids 138 to 253 of SEQ ID NO:28 and a second conserved domain which is 100% identical to a DNA binding motif comprising amino acids 252 to 303 of SEQ ID NO:28; e) a basic helix loop helix polypeptide comprising amino acids 1 to 481 of SEQ ID NO:38; f) an auxin inducible polypeptide comprising amino acids 1 to 172 of SEQ ID NO:40; g) an F box and LRR polypeptide having at least 85% global sequence identity to SEQ ID NO:42; h) a glucosyl transferase comprising amino acids 1 to 329 of SEQ ID NO:50; i) a glucosyl transferase having at least 72% global sequence identity to SEQ ID NO:52; j) a zinc finger polypeptide selected from the group consisting of SEQ ID NO:62 and SEQ ID NO:64; k) an AAA ATPase selected from the group consisting of SEQ ID NO:66 and SEQ ID NO:68; and l) a polypeptide comprising a BTB/POZ domain and an ankyrin repeat domain and having at least 67% global sequence identity to SEQ ID NO:70, wherein the transgene confers increased nematode resistance to the plant grown from the transgenic seed.

Another embodiment of the invention relates to an expression vector comprising a promoter operably linked to a polynucleotide encoding at least one polypeptide selected from the group consisting of a) a transferase comprising amino acids 1 to 448 of SEQ ID NO:2; b) a senescence related oxidoreductase having at least 69% global sequence identity to SEQ ID NO:4; c) a histidine phosphotransfer kinase/transferase having at least 73% global sequence identity to SEQ ID NO:16; d) an AP2/EREBP polypeptide comprising a first conserved domain which is at least 94% identical to a domain comprising amino acids 138 to 253 of SEQ ID NO:28 and a second conserved domain which is 100% identical to a DNA binding motif comprising amino acids 252 to 303 of SEQ ID NO:28; e) a basic helix loop helix polypeptide comprising amino acids 1 to 481 of SEQ ID NO:38; f) an auxin inducible polypeptide comprising amino acids 1 to 172 of SEQ ID NO:40; g) an F box and LRR polypeptide having at least 85% global sequence identity to SEQ ID NO:42; h) a glucosyl transferase comprising amino acids 1 to 329 of SEQ ID NO:50; i) a glucosyl transferase having at least 72% global sequence identity to SEQ ID NO:52; j) a zinc finger polypeptide selected from the group consisting of SEQ ID NO:62 and SEQ ID NO:64; k) an AAA ATPase selected from the group consisting of SEQ ID NO:66 and SEQ ID NO:68; and l) a polypeptide comprising a BTB/POZ domain and an ankyrin repeat domain and having at least 67% global sequence identity to SEQ ID NO:70. Preferably, the promoter is a constitutive promoter. More preferably, the promoter is capable of specifically directing expression in plant roots. Most preferably, the promoter is capable of specifically directing expression in a syncytia site of a plant infected with nematodes.

In another embodiment, the invention provides a method of producing a nematode-resistant transgenic plant, wherein the method comprises the steps of: a) transforming a wild type plant cell with an expression vector comprising a promoter operably linked to a polynucleotide encoding a polypeptide selected from the group consisting of i) a transferase comprising amino acids 1 to 448 of SEQ ID NO:2; ii) a senescence related oxidoreductase having at least 69% global sequence identity to SEQ ID NO:4; iii) a histidine phosphotransfer kinase/transferase having at least 73% global sequence identity to SEQ ID NO:16; iv) an AP2/EREBP polypeptide comprising a first conserved domain which is at least 94% identical to a domain comprising amino acids 138 to 253 of SEQ ID NO:28 and a second conserved domain which is 100% identical to a DNA binding motif comprising amino acids 252 to 303 of SEQ ID NO:28; v) a basic helix loop helix polypeptide comprising amino acids 1 to 481 of SEQ ID NO:38; vi) an auxin inducible polypeptide comprising amino acids 1 to 172 of SEQ ID NO:40; vii) an F box and LRR polypeptide having at least 85% global sequence identity to SEQ ID NO:42; viii) a glucosyl transferase comprising amino acids 1 to 329 of SEQ ID NO:50; ix) a glucosyl transferase having at least 72% global sequence identity to SEQ ID NO:52; x) a zinc finger polypeptide selected from the group consisting of SEQ ID NO:62 and SEQ ID NO:64; xi) an AAA ATPase selected from the group consisting of SEQ ID NO:66 and SEQ ID NO:68; and xii) a polypeptide comprising a BTB/POZ domain and an ankyrin repeat domain and having at least 67% global sequence identity to SEQ ID NO:70; b) regenerating transgenic plants from the transformed plant cell; and c) selecting transgenic plants for increased nematode resistance as compared to a control plant of the same species.

### BRIEF DESCRIPTION OF THE DRAWINGS

FIG. 1 shows the table of SEQ ID NOs assigned to corresponding genes and promoters.

FIG. 2 shows an amino acid alignment of exemplary GmSRG1 genes. The alignment is performed in Vector NTI software suite (gap opening penalty=10, gap extension penalty=0.05, gap separation penalty=8).

FIG. 3 shows an amino acid alignment of exemplary MtHPT4 genes. The alignment is performed in Vector NTI software suite (gap opening penalty=10, gap extension penalty=0.05, gap separation penalty=8).

FIG. 4a-4b shows an amino acid alignment of exemplary GmEREBP1 genes. The alignment is performed in Vector NTI software suite (gap opening penalty=10, gap extension penalty=0.05, gap separation penalty=8).

FIG. 5 shows an amino acid alignment of exemplary F-box/LRR-repeat genes. The alignment is performed in Vector NTI software suite (gap opening penalty=10, gap extension penalty=0.05, gap separation penalty=8).

FIG. 6a-6b shows an amino acid alignment of exemplary genes GmAC30GT genes. The alignment is performed in Vector NTI software suite (gap opening penalty=10, gap extension penalty=0.05, gap separation penalty=8).

FIG. 7 shows an amino acid alignment of exemplary zinc finger genes. The alignment is performed in Vector NTI software suite (gap opening penalty=10, gap extension penalty=0.05, gap separation penalty=8).

FIG. 8 shows an amino acid alignment of exemplary ZmAAA ATPase genes. The alignment is performed in Vector NTI software suite (gap opening penalty=10, gap extension penalty=0.05, gap separation penalty=8).

FIG. 9a, 9b, 9c shows an amino acid alignment of exemplary GmNPR1-like genes. The alignment is performed in Vector NTI software suite (gap opening penalty=10, gap extension penalty=0.05, gap separation penalty=8).

### DETAILED DESCRIPTION OF THE PREFERRED EMBODIMENTS

The present invention may be understood more readily by reference to the following detailed description and the examples included herein. Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used.

As used herein, the word “or” means any one member of a particular list and also includes any combination of members of that list.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like. The transgenic plant of the invention may be male sterile or male fertile, and may further include transgenes other than those that comprise the isolated polynucleotides described herein.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation.

Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

The term “gene” is used broadly to refer to any segment of nucleic acid associated with a biological function. Thus, genes include introns and exons as in genomic sequence, or just the coding sequences as in cDNAs and/or the regulatory sequences required for their expression. For example, gene refers to a nucleic acid fragment that expresses mRNA or functional RNA, or encodes a specific protein, and which includes regulatory sequences.

The terms “polypeptide” and “protein” are used interchangeably herein to refer to a polymer of consecutive amino acid residues.

The terms “operably linked” and “in operative association with” are interchangeable and as used herein refer to the association of isolated polynucleotides on a single nucleic acid fragment so that the function of one isolated polynucleotide is affected by the other isolated polynucleotide. For example, a regulatory DNA is said to be “operably linked to” a DNA that expresses an RNA or encodes a polypeptide if the two DNAs are situated such that the regulatory DNA affects the expression of the coding DNA.

The term “promoter” as used herein refers to a DNA sequence which, when ligated to a nucleotide sequence of interest, is capable of controlling the transcription of the nucleotide sequence of interest into mRNA. A promoter is typically, though not necessarily, located 5′ (e.g., upstream) of a nucleotide of interest (e.g., proximal to the transcriptional start site of a structural gene) whose transcription into mRNA it controls, and provides a site for specific binding by RNA polymerase and other transcription factors for initiation of transcription.

The term “transcription regulatory element” as used herein refers to a polynucleotide that is capable of regulating the transcription of an operably linked polynucleotide. It includes, but not limited to, promoters, enhancers, introns, 5′ UTRs, and 3′ UTRs.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. In the present specification, “plasmid” and “vector” can be used interchangeably as the plasmid is the most commonly used form of vector. A vector can be a binary vector or a T-DNA that comprises the left border and the right border and may include a gene of interest in between. The term “expression vector” is interchangeable with the term “transgene” as used herein and means a vector capable of directing expression of a particular nucleotide in an appropriate host cell. The expression of the nucleotide can be over-expression. An expression vector comprises a regulatory nucleic acid element operably linked to a nucleic acid of interest, which is—optionally—operably linked to a termination signal and/or other regulatory element.

The term “homologs” as used herein refers to a gene related to a second gene by descent from a common ancestral DNA sequence. The term “homologs” may apply to the relationship between genes separated by the event of speciation (e.g., orthologs) or to the relationship between genes separated by the event of genetic duplication (e.g., paralogs). Homologs may be described herein in terms of percent of global sequence identity (i.e., sequence identity across the entire length of the polynucleotide or polypeptide) to a polynucleotide or polypeptide which has been shown to confer nematode resistance to a transgenic plant when transformed into a wild type plant of the same species which does not contain the transgene. Alternatively homogs may be described herein in terms of percent identity to a conserved domain within a polypeptide that confers nematode resistance to a plant. Sequence identity may be determined by any of the publicly available computer programs commonly used by those of skill in biotechnology, for example, the Vector NTI 9.0 (PC) software suite available from Invitrogen, Carlsbad, Calif.)

As used herein, the term “orthologs” refers to genes from different species, but that have evolved from a common ancestral gene by speciation. Orthologs retain the same function in the course of evolution. Orthologs encode proteins having the same or similar functions. As used herein, the term “paralogs” refers to genes that are related by duplication within a genome. Paralogs usually have different functions or new functions, but these functions may be related.

The term “conserved region” or “conserved domain” as used herein refers to a region in heterologous polynucleotide or polypeptide sequences where there is a relatively high degree of sequence identity between the distinct sequences. The “conserved region” can be identified, for example, from the multiple sequence alignment using the Clustal W algorithm.

The term “cell” or “plant cell” as used herein refers to single cell, and also includes a population of cells. The population may be a pure population comprising one cell type. Likewise, the population may comprise more than one cell type. A plant cell within the meaning of the invention may be isolated (e.g., in suspension culture) or comprised in a plant tissue, plant organ or plant at any developmental stage.

The term “true breeding” as used herein refers to a variety of plant for a particular trait if it is genetically homozygous for that trait to the extent that, when the true-breeding variety is self-pollinated, a significant amount of independent segregation of the trait among the progeny is not observed.

The term “null segregant” as used herein refers to a progeny (or lines derived from the progeny) of a transgenic plant that does not contain the transgene due to Mendelian segregation.

The term “wild type” as used herein refers to a plant cell, seed, plant component, plant tissue, plant organ, or whole plant that has not been genetically modified or treated in an experimental sense.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

The term “syncytia site” as used herein refers to the feeding site formed in plant roots after nematode infestation. The site is used as a source of nutrients for the nematodes. A syncytium is the feeding site for cyst nematodes and giant cells are the feeding sites of root knot nematodes.

Crop plants and corresponding parasitic nematodes are listed in Index of Plant Diseases in the United States (U.S. Dept. of Agriculture Handbook No. 165, 1960); Distribution of Plant-Parasitic Nematode Species in North America (Society of Nematologists, 1985); and Fungi on Plants and Plant Products in the United States (American Phytopathological Society, 1989). For example, plant parasitic nematodes that are targeted by the present invention include, without limitation, cyst nematodes and root-knot nematodes. Specific plant parasitic nematodes which are targeted by the present invention include, without limitation, Heterodera glycines, Heterodera schachtii, Heterodera avenae, Heterodera oryzae, Heterodera cajani, Heterodera trifolii, Globodera pallida, G. rostochiensis, or Globodera tabacum, Meloidogyne incognita, M. arenaria, M. hapla, M. javanica, M. naasi, M. exigua, Ditylenchus dipsaci, Ditylenchus angustus, Radopholus similis, Radopholus citrophilus, Helicotylenchus multicinctus, Pratylenchus coffeae, Pratylenchus brachyurus, Pratylenchus vulnus, Paratylenchus curvitatus, Paratylenchus zeae, Rotylenchulus reniformis, Paratrichodorus anemones, Paratrichodorus minor, Paratrichodorus christiei, Anguina tritici, Bidera avenae, Subanguina radicicola, Hoplolaimus seinhorsti, Hoplolaimus Columbus, Hoplolaimus galeatus, Tylenchulus semipenetrans, Hemicycliophora arenaria, Rhadinaphelenchus cocophilus, Belonolaimus longicaudatus, Trichodorus primitivus, Nacobbus aberrans, Aphelenchoides besseyi, Hemicriconemoides kanayaensis, Tylenchorhynchus claytoni, Xiphinema americanum, Cacopaurus pestis, Heterodera zeae, Heterodera filipjevi and the like.

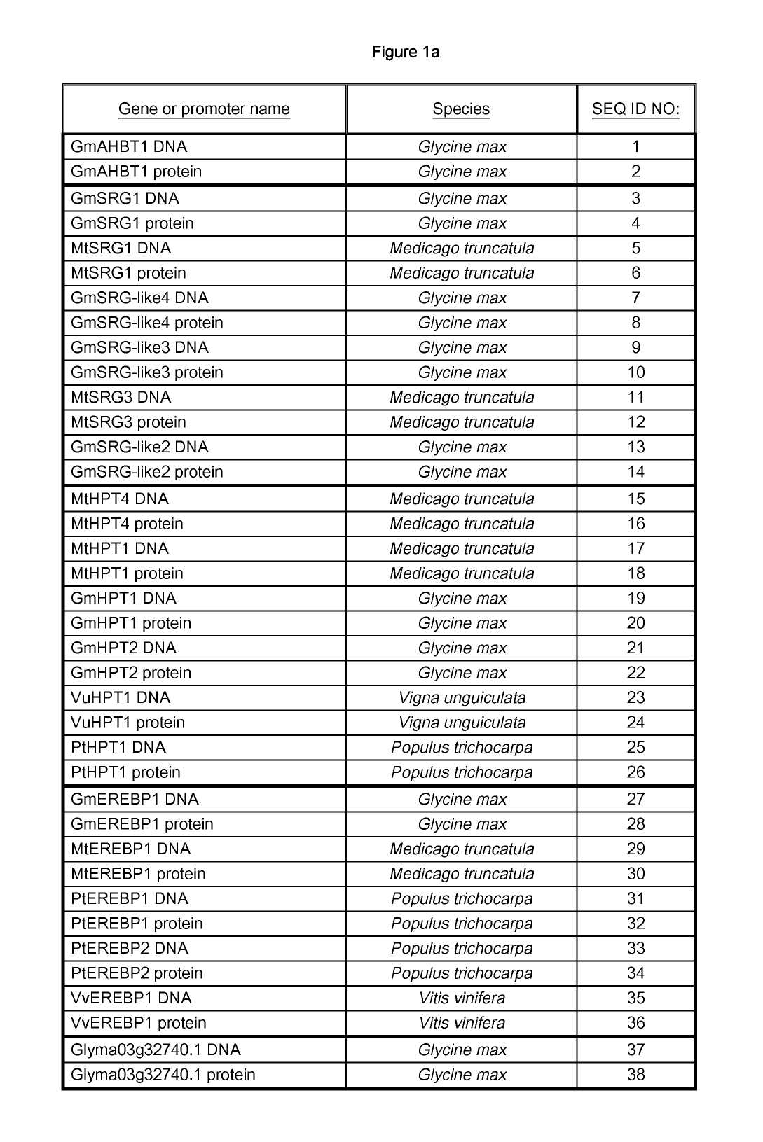

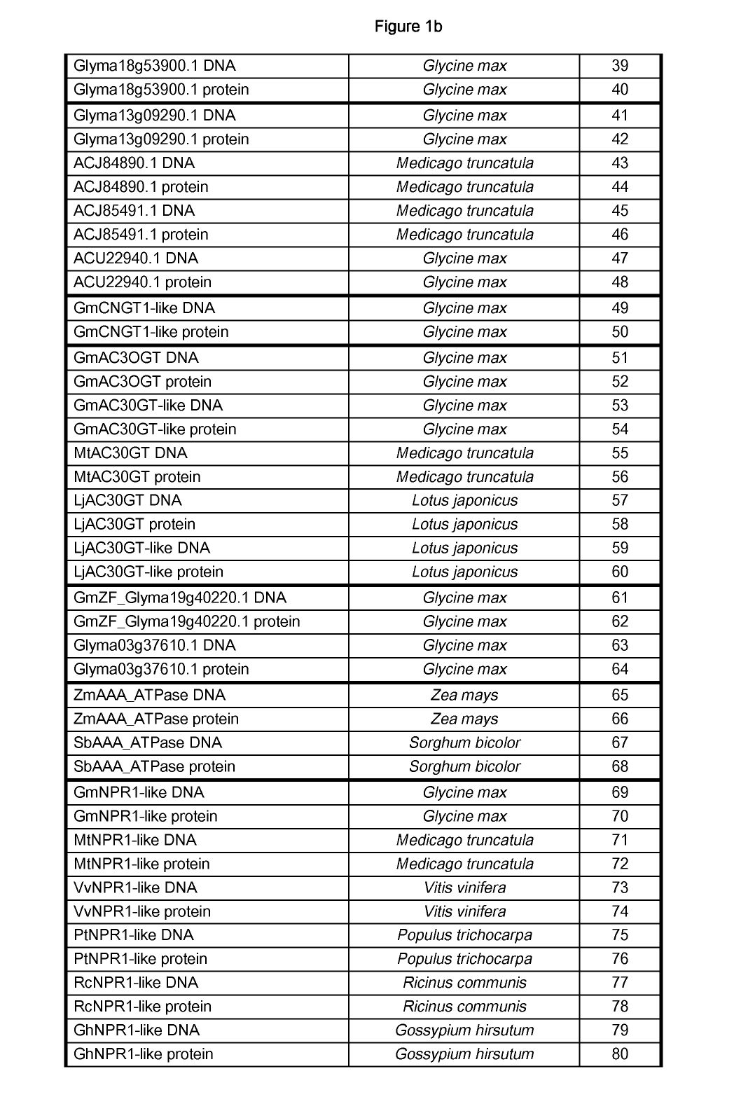

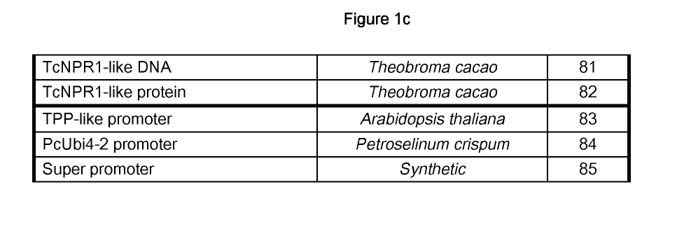

In one embodiment, the invention provides a nematode-resistant transgenic plant transformed with an expression vector comprising an isolated polynucleotide that encodes the transferase set forth in SEQ ID NO:2. The gene designated GmAHBT1 (SEQ ID NO:1) in FIG. 1 encodes a transferase protein from Glycine max, containing the conserved pfam02458 domain found in the gene superfamily that includes anthranilate N-hydroxycinnamoyl/benzoyltransferase (AHBT), shikimate O-hydroxycinnamoyltransferase and deacetylvindoline 4-O-acetyltransferase. Transferases in this gene family are involved in the secondary metabolism of a wide range of compounds, including monolignols, phytoalexins and alkaloids. As described in Examples 1 and 2 below, transgenic soybean root lines expressing the transferase polynucleotide encoding the polypeptide comprising amino acids 1 to 448 of SEQ ID NO:2 demonstrated increased resistance to nematode infection as compared to control lines.

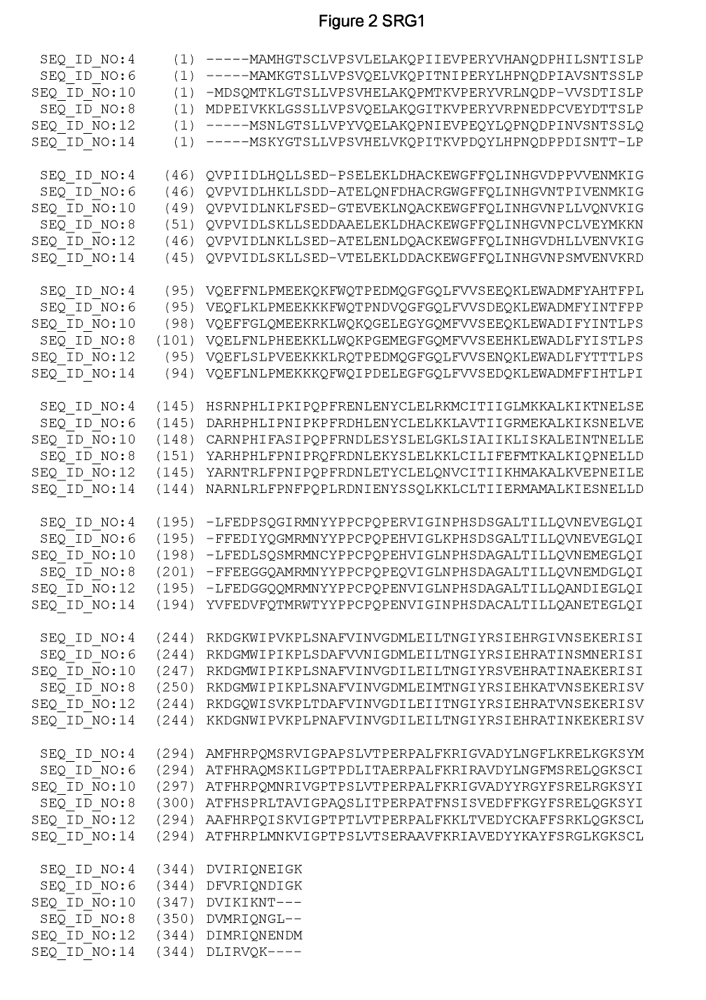

In another embodiment, the invention provides a nematode-resistant transgenic plant transformed with an expression vector comprising an isolated polynucleotide that encodes a senescence related oxidoreductase having at least 69% global sequence identity to the polypeptide set forth in SEQ ID NO:4. The gene designated GmSRG1 (SEQ ID NO:4) in FIG. 1 is a G. max gene that belongs to the 2OG-Fe(II) oxygenase superfamily. It contains the conserved pfam03171 domain characteristic of 2OG-Fe(II) oxygenase enzymes, such as 2-oxoglutarate-dependent dioxygenase, gibberellin 2-oxidase and flavonol synthase. GmSRG1 has sequence similarity to SRG1 from Arabidopsis thaliana, a senescence-associated oxidoreductase. As described in Examples 1 and 2 below, transgenic soybean root lines expressing the G. max senescence related oxidoreductase polynucleotide having SEQ ID NO:3 demonstrated increased resistance to nematode infection as compared to control lines. Several homologs of the senescence-associated oxidoreductase of SEQ ID NO:4 have been identified and described in Example 3, and an amino acid alignment of those homologs, which are suitable for use in this embodiment is set forth in FIG. 2. Any polynucleotide encoding a protein having at least 69% global sequence identity to SEQ ID NO:4 is suitable for producing a nematode-resistant transgenic plant in accordance with this embodiment. For example, polynucleotides encoding the senescence-associated oxidoreductases of SEQ ID NO:6, SEQ ID NO:8, SEQ ID NO:10, SEQ ID NO:12, or SEQ ID NO:14 may be transformed into a wild-type plant to produce a nematode-resistant transgenic plant. Alternatively, any polynucleotide encoding a senescence-associated oxidoreductase which comprises a first domain having at least 78% sequence identity to amino acids 44 to 83 of SEQ ID NO:4; a second domain having at least 86% sequence identity to amino acids 118 to 138 of SEQ ID NO:4; and a third domain having at least 79% sequence identity to amino acids 196 to 297 of SEQ ID NO:4 may be transformed into a wild-type plant to produce a nematode-resistant transgenic plant.

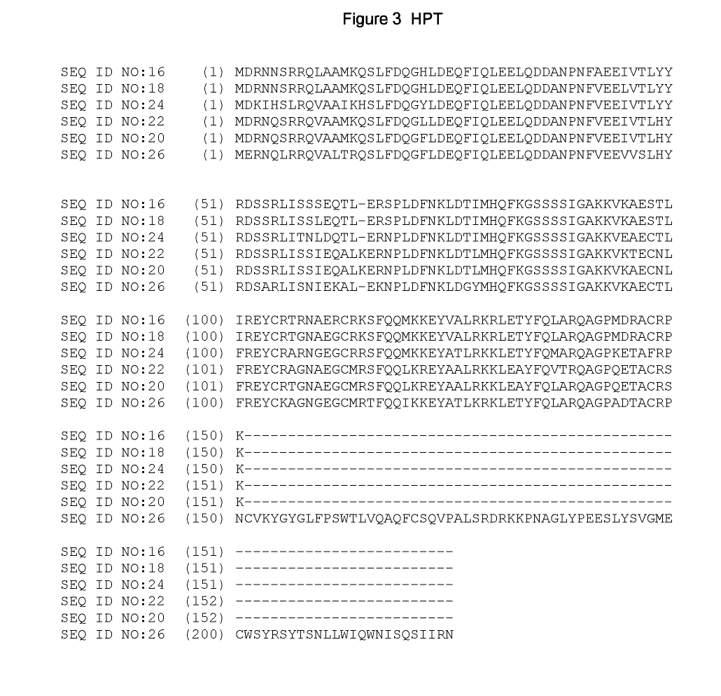

In another embodiment, the invention provides a nematode-resistant transgenic plant transformed with an expression vector comprising an isolated polynucleotide that encodes a histidine phosphotransfer kinase/transferase having at least 73% global sequence identity to the histidine phosphotransfer kinase/transferase set forth in SEQ ID NO:16: The gene designated MtHPT4 (SEQ ID NO:15) in FIG. 1 is a Medicago truncatula gene that belongs to the histidine phosphotransfer kinase/transferase gene family, which are components of multistep phosphorelay pathways. As described in Examples 1 and 2 below, transgenic soybean root lines expressing the M. truncatula histidine phosphotransfer kinase/transferase polynucleotide having SEQ ID NO:15 demonstrated increased resistance to nematode infection as compared to control lines. Several homologs of the histidine phosphotransfer kinase/transferase of SEQ ID NO:16 have been identified and described in Example 3, and an amino acid alignment of those homologs, which are suitable for use in this embodiment is set forth in FIG. 3. Any polynucleotide encoding a histidine phosphotransfer kinase/transferase having at least 73% global sequence identity to the protein of SEQ ID NO:16 is suitable for producing a nematode-resistant transgenic plant in accordance with this embodiment. For example, polynucleotides encoding the histidine phosphotransfer kinase/transferases of SEQ ID NO:18, SEQ ID NO:20, SEQ ID NO:22, SEQ ID NO:24 or SEQ ID NO:26 may be transformed into a wild-type plant to produce a nematode-resistant transgenic plant. Alternatively, any polynucleotide encoding a histidine phosphotransfer kinase/transferase comprising a first domain having at least 93% sequence identity to amino acids 16 to 44 of SEQ ID NO:16 and a second domain having at least 80% sequence identity to amino acids 51 to 100 of SEQ ID NO:16 may be transformed into a wild-type plant to produce a nematode-resistant transgenic plant.

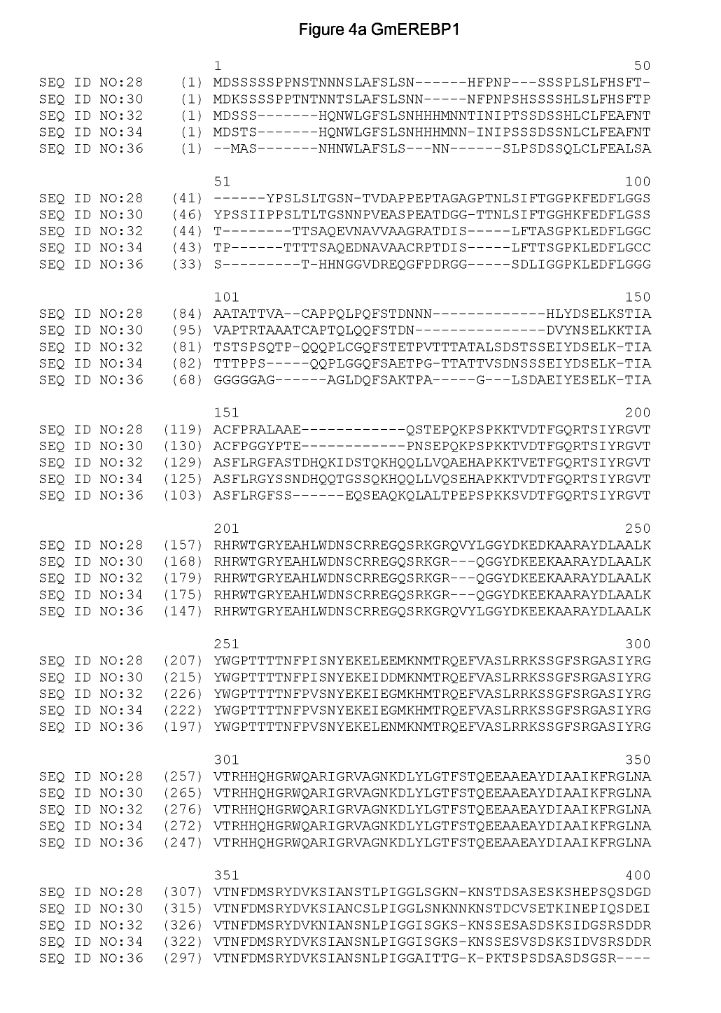

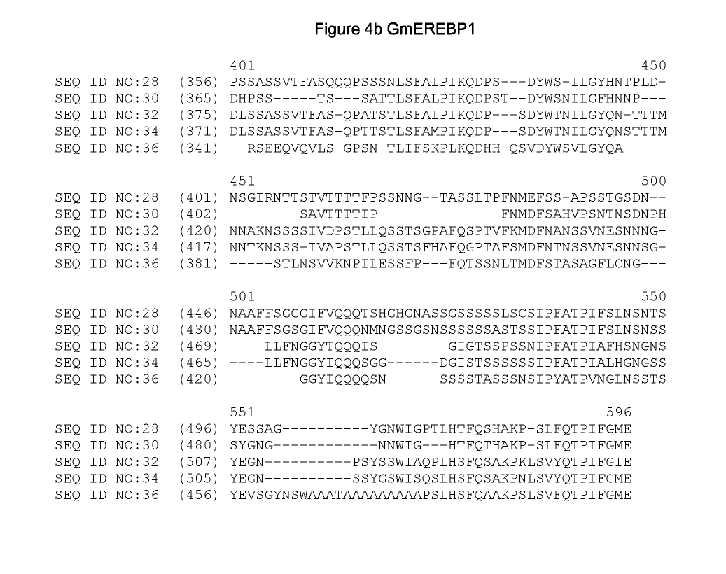

In another embodiment, the invention provides a transgenic plant transformed with an expression vector comprising an isolated polynucleotide that encodes a AP2/EREBP transcription factor comprising a first conserved domain which is at least 94% identical to a domain comprising amino acids 138 to 253 of SEQ ID NO:28 and a second conserved domain which is 100% identical to a DNA binding motif comprising amino acids 252 to 303 of SEQ ID NO:28 The gene designated GmEREBP1 (SEQ ID NO:27) in FIG. 1 encodes an AP2-domain containing protein from G. max. The AP2 proteins are a large family of DNA binding transcription factors that regulate the expression of other genes. AP2 domain containing proteins studied in plants have been implicated in a wide range of cellular processes including development, stress response, and hormone response. The GmEREBP1 protein of SEQ ID NO:28 has homology to a family of Ethylene Response Element Binding Proteins (EREBP) involved with response to the plant hormone ethylene. As described in Examples 1 and 2 below, transgenic soybean root lines expressing the G. max AP2/EREBP transcription factor polynucleotide having SEQ ID NO:27 demonstrated increased resistance to nematode infection as compared to control lines. Several homologs of the GmEREBP1 protein of SEQ ID NO:28 have been identified and described in Example 3, and an alignment of those homologs, which are suitable for use in this embodiment, is set forth in FIG. 4. Any polynucleotide encoding a AP2/EREBP transcription factor comprising a first conserved domain which is at least 94% identical to a domain comprising amino acids 138 to 253 of SEQ ID NO:28 and a second conserved domain which is 100% identical to a DNA binding motif comprising amino acids 252 to 303 of SEQ ID NO:28 may be transformed into a wild-type plant to produce a nematode-resistant transgenic plant. For example, polynucleotides encoding the AP2/EREBP transcription factors of SEQ ID NO:30, SEQ ID NO:32, SEQ ID NO:34 or SEQ ID NO:36 may be transformed into a wild-type plant to produce a nematode-resistant transgenic plant.

In another embodiment, the invention provides a transgenic plant transformed with an expression vector comprising an isolated polynucleotide that encodes a basic helix loop helix polypeptide comprising amino acids 1 to 481 of SEQ ID NO:38. The gene designated Glyma03g32740.1 (SEQ ID NO:38) in FIG. 1 is a basic Helix Loop Helix (bHLH) E-box binding domain containing protein from G. max. The bHLH proteins are a large family of transcription factors that regulate expression of other genes. Glyma03g32740.1 contains a putative E-box binding domain which specifically binds the hexanucleotide sequence 5-CANNTG-3. As described in Examples 1 and 2 below, transgenic soybean root lines expressing the G. max basic helix loop helix polynucleotide having SEQ ID NO:37 demonstrated increased resistance to nematode infection as compared to control lines.

In another embodiment, the invention provides a transgenic plant transformed with an expression vector comprising an isolated polynucleotide that encodes an auxin inducible polypeptide comprising amino acids 1 to 172 of SEQ ID NO:40. The gene designated Glyma18g53900.1 (SEQ ID NO:40) in FIG. 1 is a member of the auxin inducible protein family from Glycine max. These small genes are expressed in response to auxin treatment and have no identified function. As described in Examples 1 and 2 below, transgenic soybean root lines expressing the G. max auxin inducible polynucleotide having SEQ ID NO:39 demonstrated increased resistance to nematode infection as compared to control lines.

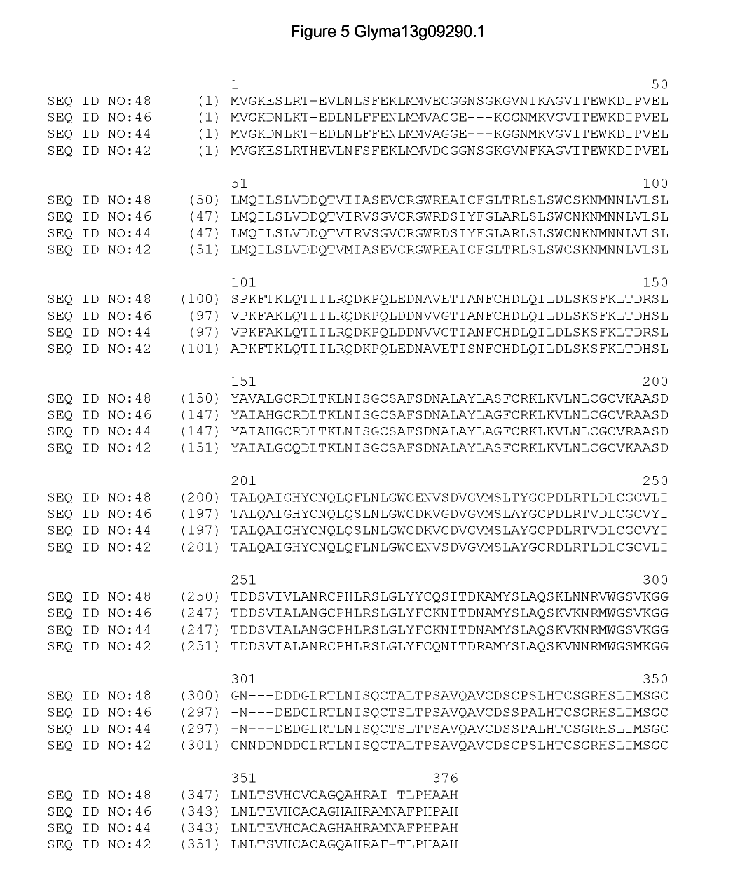

In another embodiment, the invention provides a transgenic plant transformed with an expression vector comprising an isolated polynucleotide that encodes an F box LRR polypeptide having at least 85% global sequence identity to the F box LRR polypeptide set forth in SEQ ID NO:42. The gene designated Glyma13g09290.1 (SEQ ID NO:42) in FIG. 1 is an F-box domain and Leucine-Rich-Repeat (LRR) domain containing protein from G. max. As described in Examples 1 and 2 below, transgenic soybean root lines expressing the G. max F box LRR polynucleotide having SEQ ID NO:41 demonstrated increased resistance to nematode infection as compared to control lines. Several homologs of the F box LRR polypeptide of SEQ ID NO:42 have been identified and described in Example 3, and an alignment of exemplary F box LRRs suitable for use in this embodiment is set forth in FIG. 5. Any polynucleotide encoding a F box LRR polypeptide having at least 85% global sequence identity to the polypeptide of SEQ ID NO:42 may be used as described herein to produce a nematode-resistant transgenic plant. For example, polynucleotides encoding the F box LRR polypeptides of SEQ ID NO:44, SEQ ID NO:46 or SEQ ID NO:48 may be transformed into a wild-type plant to produce a nematode-resistant transgenic plant. Alternatively, an F box LRR polypeptide comprising a first domain having at least at least 89% sequence identity to amino acids 38 to 214 of SEQ ID NO:42 and a second domain having at least 94% sequence identity to amino acids 308 to 354 of SEQ ID NO:42 may be transformed into a wild-type plant to produce a nematode-resistant transgenic plant.

In another embodiment, the invention provides a transgenic plant transformed with an expression vector comprising an isolated polynucleotide that encodes a glucosyl transferase polypeptide comprising amino acids 1 to 329 of SEQ ID NO:50. The gene designated GmCNGT1-like (SEQ ID NO:50) in FIG. 1 encodes a glucosyl transferase containing protein from G. max. Although the specific function of this protein is unknown, GmCNGT1-like has some homology to cytokinin-N-glucosyl transferase proteins which convert cytokinin compounds into an inactive storage form. As described in Examples 1 and 2 below, transgenic soybean root lines expressing the G. max glucosyl transferase polynucleotide having SEQ ID NO:49 demonstrated increased resistance to nematode infection as compared to control lines.

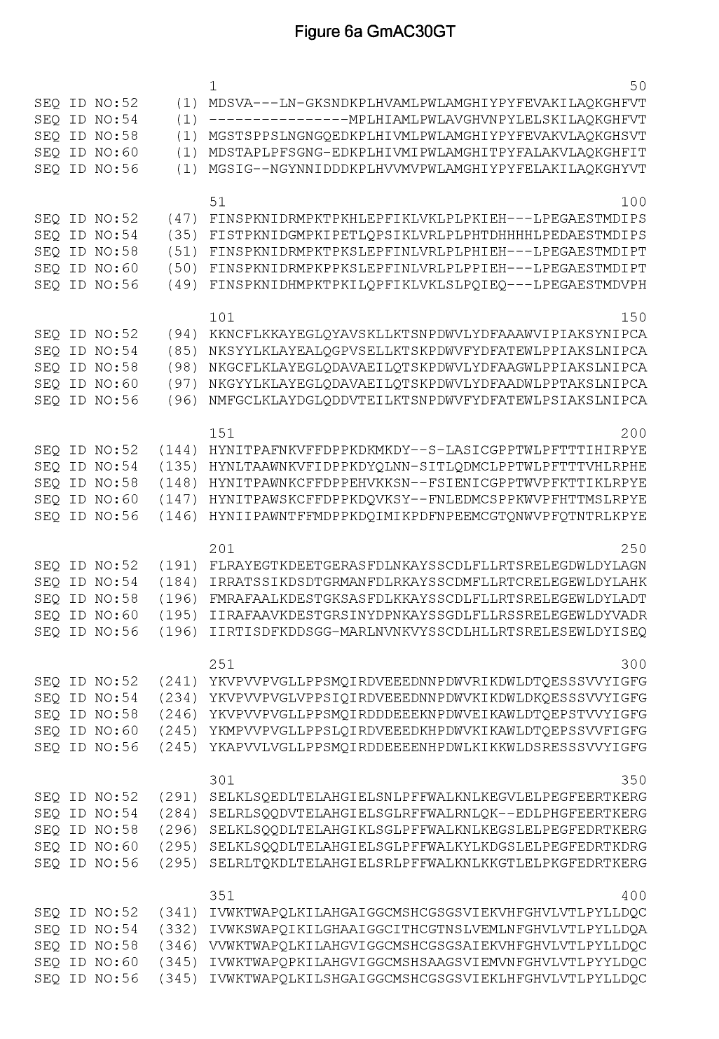

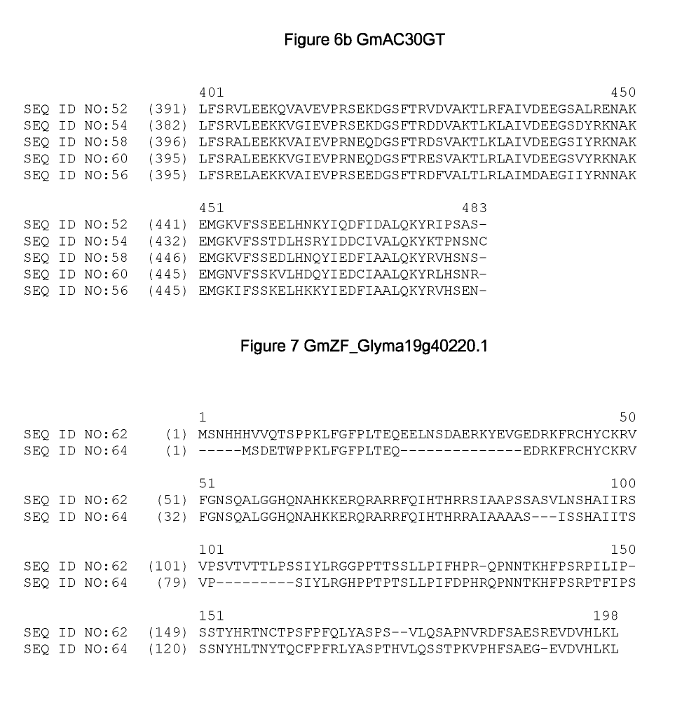

In another embodiment, the invention provides a transgenic plant transformed with an expression vector comprising an isolated polynucleotide that encodes an glucosyl transferase having at least 72% global sequence identity to the glucosyl transferase set forth in SEQ ID NO:52. The gene designated GmAC30GT (SEQ ID NO:51) in FIG. 1 encodes a UDP-glucosyl transferase containing protein (SEQ ID NO:52) from G. max. Although the specific function of the protein represented by SEQ ID NO:52 is unknown, GmAC30GT1 is homologous to anthocyanidin-3-O-glucosyl transferases involved with flavonoid biosynthesis. As described in Examples 1 and 2 below, transgenic soybean root lines expressing the G. max glucosyl transferase polynucleotide having SEQ ID NO:51 demonstrated increased resistance to nematode infection as compared to control lines. Several homologs of the glucosyl transferase of SEQ ID NO:52 have been identified and described in Example 3, and an alignment of exemplary glucosyl transferases suitable for use in this embodiment is set forth in FIG. 6. Any polynucleotide encoding a glucosyl transferase having at least 72% global sequence identity to the polypeptide of SEQ ID NO:52 may be used as described herein to produce a nematode-resistant transgenic plant. For example, a polynucleotide encoding any of the glucosyl transferases of SEQ ID NO:54, SEQ ID NO:56, SEQ ID NO:58 or SEQ ID NO:60 may be transformed into a wild-type plant to produce a nematode-resistant transgenic plant. Alternatively, a glucosyl transferase polypeptide comprising a first domain having at least 73% sequence identity to amino acids 19 to 161 of SEQ ID NO:52 a second domain having at least 83% sequence identity to amino acids 241 to 322 of SEQ ID NO:52; and a third domain having at least 77% sequence identity to amino acids 376 to 466 of SEQ ID NO:52 may be transformed into a wild-type plant to produce a nematode-resistant transgenic plant.

In another embodiment, the invention provides a transgenic plant transformed with an expression vector comprising an isolated polynucleotide that encodes a zinc finger selected from the group consisting of SEQ ID NO:62 and SEQ ID NO:64. The gene designated GmZF_Glyma19g40220.1 (SEQ ID NO:61) in FIG. 1 is a C2H2 type zinc finger containing protein from G. max. Zinc finger proteins, depending on their specific structure, are involved with a variety of cellular processes including DNA binding, protein-protein interactions, zinc binding, and RNA binding. The specific function of the GmZF_Glyma19g40220.1 polypeptide(SEQ ID NO:62) is unknown. As described in Examples 1 and 2 below, transgenic soybean root lines expressing the G. max zinc finger polynucleotide having SEQ ID NO:61 demonstrated increased resistance to nematode infection as compared to control lines. An alignment of SEQ ID NO:62 and SEQ ID NO:64 is set forth in FIG. 7. A polynucleotide encoding either of SEQ ID NO:62 or SEQ ID NO:64 may be used as described herein to produce a nematode-resistant transgenic plant.

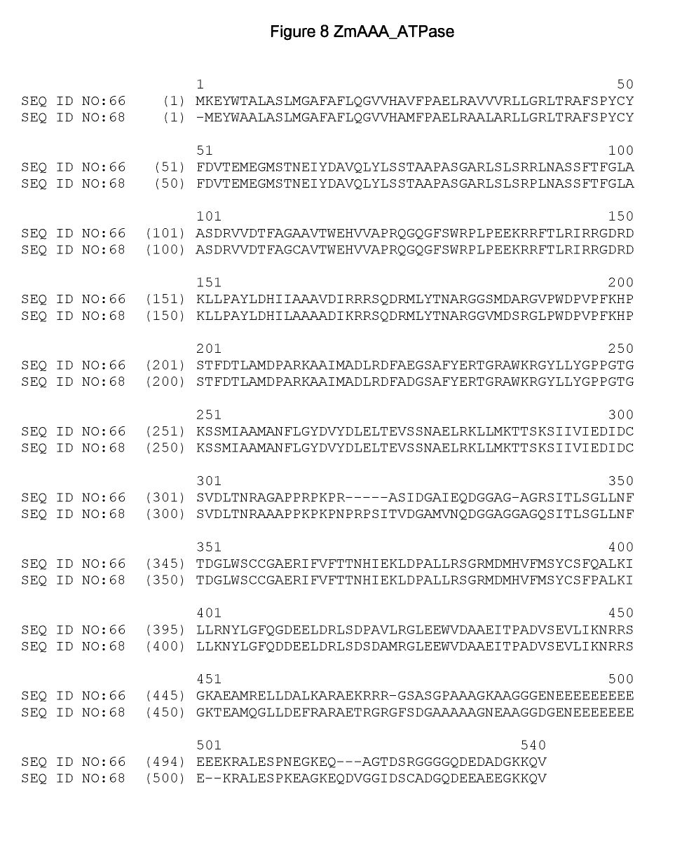

In another embodiment, the invention provides a transgenic plant transformed with an expression vector comprising an isolated polynucleotide that encodes an AAA ATPase selected from the group consisting of SEQ ID NO:66 and SEQ ID NO:68. The gene designated ZmAAA_ATPase (SEQ ID NO:65) in FIG. 1 encodes a Zea mays polypeptide (SEQ ID NO:62) containing a domain homologous to an AAA domain (ATPases Associated with diverse cellular Activities). Proteins containing this domain are involved in a variety of cellular processes including regulation of gene expression, protein modification, protein degradation, signal transduction, and other activities. The specific function of the ZmAAA_ATPase represented by SEQ ID NO:62 is unknown. As described in Examples 1 and 2 below, transgenic soybean root lines expressing the Z. mays AAA ATPase polynucleotide having SEQ ID NO:65 demonstrated increased resistance to nematode infection as compared to control lines. An alignment of SEQ ID NO:66 and SEQ ID NO:68 is set forth in FIG. 8. A polynucleotide encoding either of SEQ ID NO:66 or SEQ ID NO:68 may be transformed into a wild-type plant to produce a nematode-resistant transgenic plant.

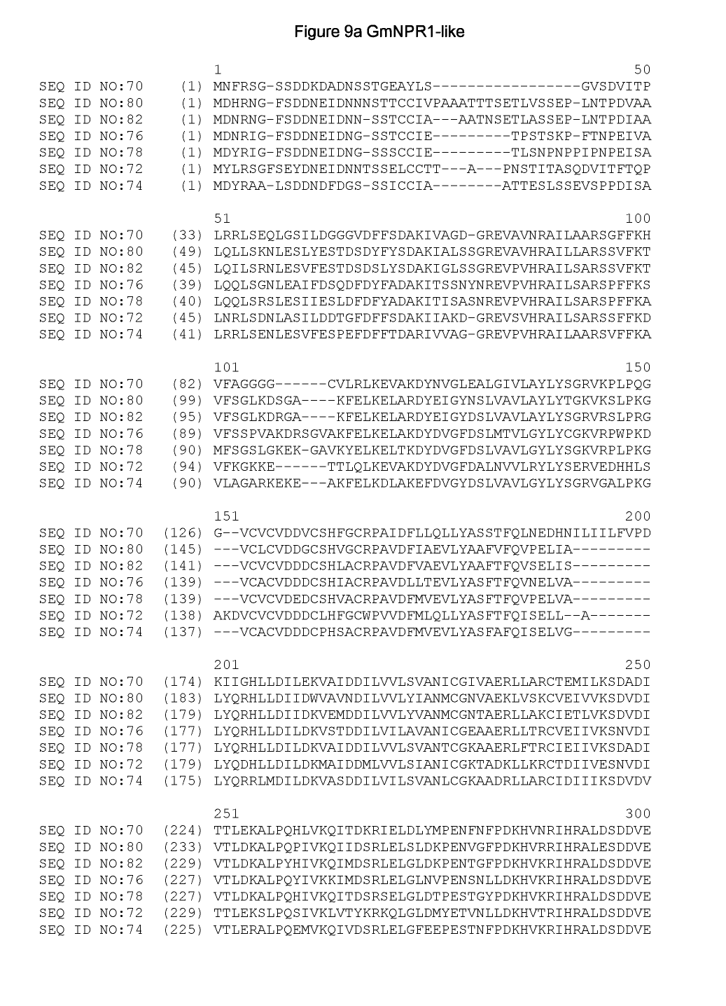

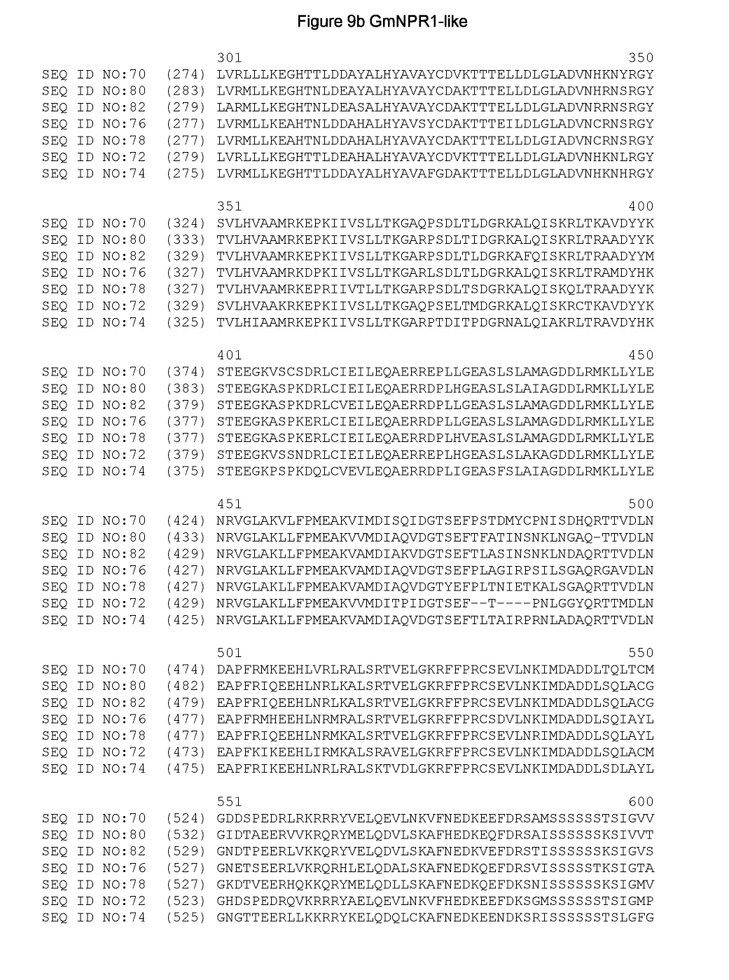

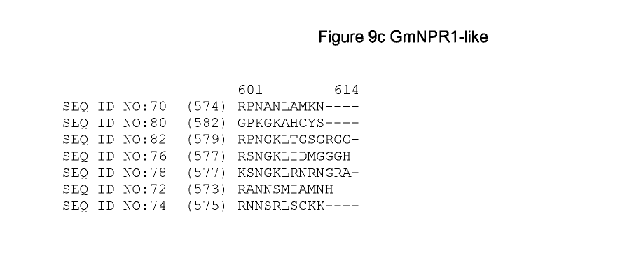

In another embodiment, the invention provides a transgenic plant transformed with an expression vector comprising an isolated polynucleotide that encodes a polypeptide comprising a BTB/POZ domain and an ankyrin repeat domain and having at least 67% global sequence identity to SEQ ID NO:70. The gene designated GmNPR1-like (SEQ ID NO:69) in FIG. 1 encodes a G. max polypeptide containing a BTB/POZ domain and an ankyrin repeat domain. BTB/POZ domains are responsible for protein interactions. Proteins containing the BTB/POZ domain have the potential to self-interact as well as interact with proteins not containing the domain. Proteins containing the BTB/POZ domain are involved with a variety of cellular functions. Ankyrin repeat domains mediate protein-protein interactions and are one of the most common domains found in proteins in nature. The GmNPR1-like gene has low homology to the Arabidopsis NPR1 gene, which is a key regulator of salicylic acid (SA) mediated plant defense signaling. As described in Examples 1 and 2 below, transgenic soybean root lines expressing the polynucleotide of SEQ ID NO:69 demonstrated increased resistance to nematode infection as compared to control lines. Several homologs of the polypeptide of SEQ ID NO:70 have been identified and described in Example 3, and an alignment of those homologs, which are suitable for use in this embodiment, is set forth in FIG. 9. Any polynucleotide encoding a polypeptide comprising a BTB/POZ domain and an ankyrin repeat domain and having at least 67% global sequence identity to the proteins of SEQ ID NO:70 may be used as described herein to produce a nematode-resistant transgenic plant. For example, a polynucleotide encoding a polypeptide selected from the group consisting of SEQ ID NO:72, SEQ ID NO:74, SEQ ID NO:76, SEQ ID NO:78, SEQ ID NO:80 or SEQ ID NO:82 may be transformed into a wild-type plant to produce a nematode-resistant transgenic plant. Alternatively, a polypeptide comprising a first domain having at least 86% sequence identity to amino acids 257 to 346 of SEQ ID NO:70, a second domain having at least 86% sequence identity to amino acids 386 to 443 of SEQ ID NO:70, and a third domain having at least 83% sequence identity to amino acids 470 to 517 of SEQ ID NO:70 may be transformed into a wild-type plant to produce a nematode-resistant transgenic plant.

In accordance with the invention, the plant may be selected from the group consisting of monocotyledonous plants and dicotyledonous plants. The plant can be from a genus selected from the group consisting of maize, wheat, rice, barley, oat, rye, sorghum, banana, and ryegrass. The plant can be from a genus selected from the group consisting of pea, alfalfa, soybean, carrot, celery, tomato, potato, cotton, tobacco, pepper, oilseed rape, sugar beet, cabbage, cauliflower, broccoli, lettuce and Arabidopsis thaliana.

The present invention also provides a plant, seed and parts from such a plant, and progeny plants from such a plant, including hybrids and inbreds. The invention also provides a method of plant breeding, e.g., to prepare a crossed fertile transgenic plant. The method comprises crossing a fertile transgenic plant comprising a particular expression vector of the invention with itself or with a second plant, e.g., one lacking the particular expression vector, to prepare the seed of a crossed fertile transgenic plant comprising the particular expression vector. The seed is then planted to obtain a crossed fertile transgenic plant. The plant may be a monocot. The crossed fertile transgenic plant may have the particular expression vector inherited through a female parent or through a male parent. The second plant may be an inbred plant. The crossed fertile transgenic may be a hybrid. Also included within the present invention are seeds of any of these crossed fertile transgenic plants.

The transgenic plants of the invention may be crossed with similar transgenic plants or with transgenic plants lacking the nucleic acids of the invention or with non-transgenic plants, using known methods of plant breeding, to prepare seeds. Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The crossed fertile transgenic plant may have the particular expression cassette inherited through a female parent or through a male parent. The second plant may be an inbred plant. The crossed fertile transgenic may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. In accordance with the invention, nematode resistance may be enhanced by stacking the genes disclosed herein with each other or with other genes or expression vectors capable of conferring some level of nematode resistance. These stacked combinations can be created by any method, including but not limited to cross breeding plants by conventional methods or by genetic transformation. If the traits are stacked by genetic transformation, the stacked genes can be combined sequentially or simultaneously in any order. For example if two genes are to be introduced, the two sequences can be contained in separate transformation cassettes or on the same transformation cassette. The expression of the sequences can be driven by the same or different promoters.

Another embodiment of the invention relates to an expression vector comprising a promoter operably linked to one or more polynucleotides of the invention, wherein expression of the polynucleotide confers increased nematode resistance to a transgenic plant. In one embodiment, the transcription regulatory element is a promoter capable of regulating constitutive expression of an operably linked polynucleotide. A “constitutive promoter” refers to a promoter that is able to express the open reading frame or the regulatory element that it controls in all or nearly all of the plant tissues during all or nearly all developmental stages of the plant. Constitutive promoters include, but are not limited to, the 35S CaMV promoter from plant viruses (Franck et al., Cell 21:285-294, 1980), the Nos promoter (An G. at al., The Plant Cell 3:225-233, 1990), the ubiquitin promoter (Christensen et al., Plant Mol. Biol. 12:619-632, 1992 and 18:581-8, 1991), the MAS promoter (Velten et al., EMBO J. 3:2723-30, 1984), the maize H3 histone promoter (Lepetit et al., Mol Gen. Genet 231:276-85, 1992), the ALS promoter (WO96/30530), the 19S CaMV promoter (U.S. Pat. No. 5,352,605), the super-promoter (U.S. Pat. No. 5,955,646), the figwort mosaic virus promoter (U.S. Pat. No. 6,051,753), the rice actin promoter (U.S. Pat. No. 5,641,876), and the Rubisco small subunit promoter (U.S. Pat. No. 4,962,028).

In another embodiment, the transcription regulatory element is a regulated promoter. A “regulated promoter” refers to a promoter that directs gene expression not constitutively, but in a temporally and/or spatially manner, and includes both tissue-specific and inducible promoters. Different promoters may direct the expression of a gene or regulatory element in different tissues or cell types, or at different stages of development, or in response to different environmental conditions.

A “tissue-specific promoter” or “tissue-preferred promoter” refers to a regulated promoter that is not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of sequence. Suitable promoters include the napin-gene promoter from rapeseed (U.S. Pat. No. 5,608,152), the USP-promoter from Vicia faba (Baeumlein et al., Mol Gen Genet. 225(3):459-67, 1991), the oleosin-promoter from Arabidopsis (WO 98/45461), the phaseolin-promoter from Phaseolus vulgaris (U.S. Pat. No. 5,504,200), the Bce4-promoter from Brassica (WO 91/13980) or the legumin B4 promoter (LeB4; Baeumlein et al., Plant Journal, 2(2):233-9, 1992) as well as promoters conferring seed specific expression in monocot plants like maize, barley, wheat, rye, rice, etc. Suitable promoters to note are the Ipt2 or Ipt1-gene promoter from barley (WO 95/15389 and WO 95/23230) or those described in WO 99/16890 (promoters from the barley hordein-gene, rice glutelin gene, rice oryzin gene, rice prolamin gene, wheat gliadin gene, wheat glutelin gene, maize zein gene, oat glutelin gene, Sorghum kasirin-gene and rye secalin gene). Promoters suitable for preferential expression in plant root tissues include, for example, the promoter derived from corn nicotianamine synthase gene (US 20030131377) and rice RCC3 promoter (U.S. Ser. No. 11/075,113). Suitable promoter for preferential expression in plant green tissues include the promoters from genes such as maize aldolase gene FDA (US 20040216189), aldolase and pyruvate orthophosphate dikinase (PPDK) (Taniguchi et. al., Plant Cell Physiol. 41(1):42-48, 2000).

Inducible promoters” refer to those regulated promoters that can be turned on in one or more cell types by an external stimulus, for example, a chemical, light, hormone, stress, or a nematode such as nematodes. Chemically inducible promoters are especially suitable if gene expression is wanted to occur in a time specific manner. Examples of such promoters are a salicylic acid inducible promoter (WO 95/19443), a tetracycline inducible promoter (Gatz et al., Plant J. 2:397-404, 1992), the light-inducible promoter from the small subunit of Ribulose-1,5-bis-phosphate carboxylase (ssRUBISCO), and an ethanol inducible promoter (WO 93/21334). Also, suitable promoters responding to biotic or abiotic stress conditions are those such as the nematode inducible PRP1-gene promoter (Ward et al., Plant. Mol. Biol. 22:361-366, 1993), the heat inducible hsp80-promoter from tomato (U.S. Pat. No. 5,187,267), cold inducible alpha-amylase promoter from potato (WO 96/12814), the drought-inducible promoter of maize (Busk et. al., Plant J. 11:1285-1295, 1997), the cold, drought, and high salt inducible promoter from potato (Kirch, Plant Mol. Biol. 33:897-909, 1997) or the RD29A promoter from Arabidopsis (Yamaguchi-Shinozalei et. al., Mol. Gen. Genet. 236:331-340, 1993), many cold inducible promoters such as cor15a promoter from Arabidopsis (Genbank Accession No U01377), blt101 and blt4.8 from barley (Genbank Accession Nos AJ310994 and U63993), wcs120 from wheat (Genbank Accession No AF031235), mlip15 from corn (Genbank Accession No D26563), bn115 from Brassica (Genbank Accession No U01377), and the wound-inducible pinII-promoter (European Patent No. 375091).

Of particular utility in the present invention are syncytia site preferred, or nematode feeding site induced, promoters, including, but not limited to promoters from the Mtn3-like promoter disclosed in WO 2008/095887, the Mtn21-like promoter disclosed in WO 2007/096275, the peroxidase-like promoter disclosed in WO 2008/077892, the trehalose-6-phosphate phosphatase-like promoter disclosed in WO 2008/071726 and the At5g12170-like promoter disclosed in WO 2008/095888. All of the forgoing applications are incorporated herein by reference.

Yet another embodiment of the invention relates to a method of producing a nematode-resistant transgenic plant, wherein the method comprises the steps of: a) transforming a wild-type plant with an expression vector comprising a polynucleotide encoding a; and c) selecting transgenic plants for increased nematode resistance.

A variety of methods for introducing polynucleotides into the genome of plants and for the regeneration of plants from plant tissues or plant cells are known in, for example, Plant Molecular Biology and Biotechnology (CRC Press, Boca Raton, Fla.), chapter 6/7, pp. 71-119 (1993); White F F (1993) Vectors for Gene Transfer in Higher Plants; Transgenic Plants, vol. 1, Engineering and Utilization, Ed.: Kung and Wu R, Academic Press, 15-38; Jenes B et al. (1993) Techniques for Gene Transfer; Transgenic Plants, vol. 1, Engineering and Utilization, Ed.: Kung and R. Wu, Academic Press, pp. 128-143; Potrykus (1991) Annu Rev Plant Physiol Plant Molec Biol 42:205-225; Halford N G, Shewry P R (2000) Br Med Bull 56(1):62-73.

Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm M E et al., Bio/Technology. 8(9):833-9, 1990; Gordon-Kamm et al. Plant Cell 2:603, 1990), electroporation, incubation of dry embryos in DNA-comprising solution, and microinjection. In the case of these direct transformation methods, the plasmids used need not meet any particular requirements. Simple plasmids, such as those of the pUC series, pBR322, M13 mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229. The Agrobacterium-mediated transformation is best suited to dicotyledonous plants but has also been adapted to monocotyledonous plants. The transformation of plants by Agrobacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225.

The nucleotides described herein can be directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit high expression levels. In one embodiment, the nucleotides are inserted into a plastid targeting vector and transformed into the plastid genome of a desired plant host. Plants homoplasmic for plastid genomes containing the nucleotide sequences are obtained, and are preferentially capable of high expression of the nucleotides.

Plastid transformation technology is for example extensively described in U.S. Pat. NOs. 5,451,513, 5,545,817, 5,545,818, and 5,877,462 in WO 95/16783 and WO 97/32977, and in McBride et al. (1994) PNAS 91, 7301-7305.

The transgenic plants of the invention may be used in a method of controlling infestation of a crop by a plant nematode, which comprises the step of growing said crop from seeds comprising an expression vector comprising a promoter operably linked to a polynucleotide encoding at least one polynucleotide encoding a polypeptide selected from the group consisting of a) a transferase comprising amino acids 1 to 448 of SEQ ID NO:2; b) a senescence related oxidoreductase having at least 69% global sequence identity to SEQ ID NO:4; c) a histidine phosphotransfer kinase/transferase having at least 73% global sequence identity to SEQ ID NO:16; d) an AP2/EREBP polypeptide comprising a first conserved domain which is at least 94% identical to a domain comprising amino acids 138 to 253 of SEQ ID NO:28 and a second conserved domain which is 100% identical to a DNA binding motif comprising amino acids 252 to 303 of SEQ ID NO:28; e) a basic helix loop helix polypeptide comprising amino acids 1 to 481 of SEQ ID NO:38; f) an auxin inducible polypeptide comprising amino acids 1 to 172 of SEQ ID NO:40; g) an F box and LRR polypeptide having at least 85% global sequence identity to SEQ ID NO:42; h) a glucosyl transferase comprising amino acids 1 to 329 of SEQ ID NO:50; i) a glucosyl transferase having at least 72% global sequence identity to SEQ ID NO:52; j) a zinc finger polypeptide selected from the group consisting of SEQ ID NO:62 and SEQ ID NO:64; k) an AAA ATPase selected from the group consisting of SEQ ID NO:66 and SEQ ID NO:68; and l) a polypeptide comprising a BTB/POZ domain and an ankyrin repeat domain and having at least 67% global sequence identity to SEQ ID NO:70, wherein the expression vector is stably integrated into the genomes of the seeds.

The invention is further illustrated by the following examples, which are not to be construed in any way as imposing limitations upon the scope thereof.

### Example 1

### Vector Construction

PCR was used to isolate DNA fragments used to construct the binary vectors described in Table 1 and discussed in Example 2. The PCR products were cloned into TOPO pCR2.1 vectors (Invitrogen, Carlsbad, Calif.), and inserts were confirmed by sequencing. Open reading frames described by the genes designated GmAHBT1 (SEQ ID NO:1), GmSRG1 (SEQ ID NO:3), MtHPT4 (SEQ ID NO:15), GmEREBP1 (SEQ ID NO:27), Glyma03g32740.1 (SEQ ID NO:37), Glyma18g53900.1 (SEQ ID NO:39), Glyma13g09290.1 (SEQ ID NO:41), GmCNGT1-like (SEQ ID NO:49), GmAC30GT (SEQ ID NO:51), GmZF_Glyma19g40220.1 (SEQ ID NO:61) and ZmAAA_ATPase (SEQ ID NO:65) were isolated using this method.

The GmNPR1-like gene (SEQ ID NO:69) was synthesized to construct the binary vectors described in Table 1 and discussed in Example 2 and Example 3. The synthesized DNA sequence was cloned into a TOPO pCR2.1 vector (Invitrogen, Carlsbad, Calif.), and the insert was confirmed by sequencing.

The cloned GmSRG1 (SEQ ID NO:3), GmZF_Glyma19g40220.1 (SEQ ID NO:59) and GmNPR1-like (SEQ ID NO:69) genes were sequenced and individually subcloned into a plant expression vector containing a TPP promoter from A. thaliana (WO 2008/071726; p-AtTPP promoter (SEQ ID NO:83) in FIG. 1). The cloned GmAHBT1 (SEQ ID NO:1) was sequenced and individually subcloned into a plant expression vector containing a Ubiquitin promoter from parsley (WO 03/102198; p-PcUbi4-2 promoter (SEQ ID NO:84) in FIG. 1). The cloned GmSRG1 (SEQ ID NO:3), MtHPT4 (SEQ ID NO:15), GmEREBP1 (SEQ ID NO:27), Glyma03g32740.1 (SEQ ID NO:37), Glyma18g53900.1 (SEQ ID NO:39), Glyma13g09290.1 (SEQ ID NO:41), GmCNGT1-like (SEQ ID NO:49), GmAC30GT (SEQ ID NO:51) and GmZF_Glyma19g40220.1 (SEQ ID NO:61) and ZmAAA_ATPase (SEQ ID NO:65) genes were sequenced and individually subcloned into a plant expression vector containing the SUPER promoter (U.S. Pat. No. 5,955,646) (SEQ ID NO:85 in FIG. 1). The selection marker for transformation was the mutated form of the acetohydroxy acid synthase (AHAS) selection gene (also referred to as AHAS2) from Arabidopsis thaliana (Sathasivan et al., Plant Phys. 97:1044-50, 1991), conferring resistance to the herbicide ARSENAL (Imazapyr, BASF Corporation, Mount Olive, N.J.). The expression of AHAS2 was driven by a ubiquitin promoter from parsley (WO 03/102198) (SEQ ID NO:84). Table 1 describes the constructs containing GmAHBT1, GmSRG1, MtHPT4, GmEREBP1, Glyma03g32740.1, Glyma18g53900.1, Glyma13g09290.1, GmCNGT1-like, GmAC30GT, GmZF_Glyma19g40220.1, ZmAAA_ATPase and GmNPR1-like genes.

TABLE 1

| | | | Gene SEQ |
|---|---|---|---|
| Vector Name | Promoter Name | Gene Name | ID NO: |
| RTP4221-1 | PcUbi4-2 | GmAHBT1 | 1 |
| RTP1897-1 | AtTPP | GmSRG1 | 3 |
| RTP3859-1 | Super | GmSRG1 | 3 |
| RTP5960-3 | Super | MtHPT4 | 15 |
| RTP2771-1 | Super | GmEREBP1 | 27 |
| RTP5834-1 | Super | Glyma03g32740.1 | 37 |
| RTP5848-1 | Super | Glyma18g53900.1 | 39 |
| RTP5958-1 | Super | Glyma13g09290.1 | 41 |
| RTP3857-2 | Super | GmCNGT1-like | 49 |
| RTP2830-1 | Super | GmAC30GT | 51 |
| RTP4931-1 | Super | GmZF_Glyma19g40220.1 | 61 |
| RTP4932-1 | AtTPP | GmZF_Glyma19g40220.1 | 61 |
| RTP4453-1 | Super | ZmAAA_ATPase | 65 |
| RTP4926-1 | AtTPP | GmNPR1-like | 69 |

### Example 2

### Nematode Bioassay

A bioassay to assess nematode resistance conferred by the polynucleotides described herein was performed using a rooted plant assay system disclosed in commonly owned copending U.S. Pat. Pub. 2008/0153102. Transgenic roots were generated after transformation with the binary vectors described in Example 1. Multiple transgenic root lines were sub-cultured and inoculated with surface-decontaminated race 3 SCN second stage juveniles (J2) at the level of about 500 J2/well. Four weeks after nematode inoculation, the cyst number in each well was counted. For each transformation construct, the number of cysts per line was calculated to determine the average cyst count and standard error for the construct. The cyst count values for each transformation construct was compared to the cyst count values of an empty vector control tested in parallel to determine if the construct tested results in a reduction in cyst count. Rooted explant cultures transformed with vectors RTP4221-1, RTP1897-1, RTP3859-1, RTP5960-3, RTP2771-1, RTP5834-1, RTP5848-1, RTP5958-1, RTP3857-2, RTP2830-1, RTP4931-1, RTP4932-1, RTP4453-1 and RTP4926-1 exhibited a general trend of reduced cyst numbers and female index relative to the known susceptible variety, Williams82.

### Example 3

### Homolog Identification and Description

As disclosed in Example 2, expressing a GmSRG1 transcript contained in vectors RTP1897-1 or RTP3859-1 results in reduced cyst counts when operably linked to a Super or AtTPP promoter and expressed in soybean roots. As disclosed in Example 1, the transcript contains an open reading frame with DNA sequences disclosed as SEQ ID NO:3 and the amino acid sequences disclosed as SEQ ID NO:4. The amino acid sequences described by SEQ ID NO:4 were used to identify similar genes from soybean and other plant species described by SEQ ID NO: 6, 8, 10, 12, and 14 with corresponding DNA open reading frame sequences described by SEQ ID NO:5, 7, 9, 11, 13. The amino acid alignment to SEQ ID NO:4 is shown in FIG. 2. The global percent identity between SEQ ID NO:4 and SEQ ID NO:6 is 75%, the global percent identity between SEQ ID NO:4 and SEQ ID NO:8 is 69%, the global percent identity between SEQ ID NO:4 and SEQ ID NO:10 is 73%, the global percent identity between SEQ ID NO:4 and SEQ ID NO:12 is 72%, and the global percent identity between SEQ ID NO:4 and SEQ ID NO:14 is 69%. Based on the amino acid alignment in FIG. 2, there are three regions of high amino acid similarity among SEQ ID NO:4, 6, 8, 10, 12 and 14. The first conserved domain, corresponding to the region between amino acid 44 through amino acid 83 in SEQ ID NO:4, is 100% identical between SEQ ID NO:4 and SEQ ID NO:6, 88% identical between SEQ ID NO:4 and SEQ ID NO:8, 78% identical between SEQ ID NO:4 and SEQ ID NO:10, 80% identical between SEQ ID NO:4 and SEQ ID NO:12 and 85% identical between SEQ ID NO:4 and SEQ ID NO:14. The second conserved domain, corresponding to the region between amino acid 118 through amino acid 138 in SEQ ID NO:4, is 95% identical between SEQ ID NO:4 and SEQ ID NO:6, 86% identical between SEQ ID NO:4 and SEQ ID NO:8, 86% identical between SEQ ID NO:4 and SEQ ID NO:10, 90% identical between SEQ ID NO:4 and SEQ ID NO:12 and 90% identical between SEQ ID NO:4 and SEQ ID NO:14. The third conserved domain, corresponding to the region between amino acid 196 through amino acid 297 in SEQ ID NO:4, is 83% identical between SEQ ID NO:4 and SEQ ID NO:6, 82% identical between SEQ ID NO:4 and SEQ ID NO:8, 83% identical between SEQ ID NO:4 and SEQ ID NO:10, 80% identical between SEQ ID NO:4 and SEQ ID NO:12 and 79% identical between SEQ ID NO:4 and SEQ ID NO:14.

As disclosed in Example 2, expressing a MtHPT4 transcript contained in vector RTP5960-3 results in reduced cyst counts when operably linked to a Super promoter and expressed in soybean roots. As disclosed in Example 1, the transcript contains an open reading frame with DNA sequence disclosed as SEQ ID NO:15 and the amino acid sequence disclosed as SEQ ID NO:16. The amino acid sequence described by SEQ ID NO:16 was used to identify similar genes from soybean and other plant species described by SEQ ID NO:18, 20, 22, 24, and 26 with corresponding DNA open reading frame sequences described by SEQ ID NO:17, 19, 21, 23, and 25. The amino acid alignment to SEQ ID NO:16 is shown in FIG. 3. The global percent identity between SEQ ID NO:16 and SEQ ID NO:18 is 97%, the global percent identity between SEQ ID NO:16 and SEQ ID NO:20 is 83%, the global percent identity between SEQ ID NO:16 and SEQ ID NO:22 is 81%, the global percent identity between SEQ ID NO:16 and SEQ ID NO:24 is 81%, and the global percent identity between SEQ ID NO:26 and SEQ ID NO:14 is 73%. Based on the amino acid alignment in FIG. 3, there are two regions of high amino acid similarity among SEQ ID NO:16, 18, 20, 22, 24 and 26. The first conserved domain, corresponding to the region between amino acid 16 through amino acid 44 in SEQ ID NO:16, is 96% identical between SEQ ID NO:16 and SEQ ID NO:18, 93% identical between SEQ ID NO:16 and SEQ ID NO:20, 93% identical between SEQ ID NO:16 and SEQ ID NO:22, 93% identical between SEQ ID NO:16 and SEQ ID NO:24 and 93% identical between SEQ ID NO:16 and SEQ ID NO:26. The second conserved domain, corresponding to the region between amino acid 51 through amino acid 100 in SEQ ID NO:16, is 98% identical between SEQ ID NO:16 and SEQ ID NO:18, 88% identical between SEQ ID NO:16 and SEQ ID NO:20, 86% identical between SEQ ID NO:16 and SEQ ID NO:22, 86% identical between SEQ ID NO:16 and SEQ ID NO:24 and 80% identical between SEQ ID NO:16 and SEQ ID NO:26.

As disclosed in Example 2, expressing an GmEREBP1 transcript contained in vector RTP2771-1 results in reduced cyst counts when operably linked to a Super promoter and expressed in soybean roots. As disclosed in Example 1, the transcript contains an open reading frame with DNA sequence disclosed as SEQ ID NO:27 and the amino acid sequence disclosed as SEQ ID NO:28. The DNA sequence described by SEQ ID NO:28 was used to identify similar genes from other plant species described by SEQ ID NO: 29, 31, 33 and 35, with corresponding protein translations described by SEQ ID NO:30, 32, 34 and 36. The amino acid alignment to SEQ ID NO:28 is shown in FIG. 4a-b. The global percent identity between SEQ ID NO:28 and SEQ ID NO:30 is 81%, the global percent identity between SEQ ID NO:28 and SEQ ID NO:32 is 64%, the global percent identity between SEQ ID NO:28 and SEQ ID NO:34 is 63%, the global percent identity between SEQ ID NO:28 and SEQ ID NO:36 is 63%. Based on the amino acid alignment in FIG. 4, there are two regions of high amino acid similarity among SEQ ID NO:28, 30, 32, 34 and 36. The first conserved domain, corresponding to the region between amino acid 138 through amino acid 253 in SEQ ID NO:28, is 96% identical between SEQ ID NO:28 and SEQ ID NO:30, 94% identical between SEQ ID NO:28 and SEQ ID NO:32, 95% identical between SEQ ID NO:28 and SEQ ID NO:34 and 96% identical between SEQ ID NO:28 and SEQ ID NO:36. There is a region resembling an AP2 DNA binding domain in the first conserved domain corresponding to the region between amino acid 150 through amino acid 209 of SEQ ID NO:28. The second conserved domain representing a second AP2 DNA binding motif, corresponding to the region between amino acid 252 through amino acid 303 in SEQ ID NO:28, is 100% identical between SEQ ID NO:28 and SEQ ID NO:30, 100% identical between SEQ ID NO:28 and SEQ ID NO:32, 100% identical between SEQ ID NO:28 and SEQ ID NO:34 and 100% identical between SEQ ID NO:28 and SEQ ID NO:36.

As disclosed in Example 2, expressing a Glyma13g09290.1 transcript contained in vector RTP5958-1 results in reduced cyst counts when operably linked to a Super promoter and expressed in soybean roots. As disclosed in Example 1, the transcript contains an open reading frame with DNA sequence disclosed as SEQ ID NO:41 and the amino acid sequence disclosed as SEQ ID NO:42. The DNA sequence described by SEQ ID NO:41 was used to identify similar genes from soybean and other plant species described by SEQ ID NO:43, 45 and 47, with corresponding protein translation described by SEQ ID NO:44, 46 and 48. The amino acid alignment to SEQ ID NO:42 is shown in FIG. 5. The global percent identity between SEQ ID NO:42 and SEQ ID NO:44 is 85%, the global percent identity between SEQ ID NO:42 and SEQ ID NO:46 is 85%, the global percent identity between SEQ ID NO:42 and SEQ ID NO:46 is 94%. Based on the amino acid alignment in FIG. 5, there are two regions of high amino acid similarity among SEQ ID NO:42, 44, 46 and 48. The first conserved domain, corresponding to the region between amino acid 38 through amino acid 214 in SEQ ID NO:42, is 89% identical between SEQ ID NO:42 and SEQ ID NO:44, 89% identical between SEQ ID NO:42 and SEQ ID NO:46 and 97% identical between SEQ ID NO:42 and SEQ ID NO:48 and 96% identical between SEQ ID NO:28 and SEQ ID NO:36. The second conserved domain, corresponding to the region between amino acid 308 through amino acid 354 in SEQ ID NO:42, is 94% identical between SEQ ID NO:42 and SEQ ID NO:44, 94% identical between SEQ ID NO:42 and SEQ ID NO:46 and 100% identical between SEQ ID NO:42 and SEQ ID NO:48.

As disclosed in Example 2, expressing a GmAC30GT transcript contained in vector RTP2830-1 results in reduced cyst counts when operably linked to a Super promoter and expressed in soybean roots. As disclosed in Example 1, the transcript contains an open reading frame with DNA sequence disclosed as SEQ ID NO:51 and the amino acid sequence disclosed as SEQ ID NO:52. The DNA sequence described by SEQ ID NO:51 was used to identify similar genes from soybean and other plant species, described by SEQ ID NO:53, 55, 57 and 59, with corresponding protein translations described by SEQ ID NO:54, 56, 58 and 60. The amino acid alignment to SEQ ID NO:52 is shown in FIG. 6. The global percent identity between SEQ ID NO:52 and SEQ ID NO:54 is 74%, the global percent identity between SEQ ID NO:52 and SEQ ID NO:56 is 72%, the global percent identity between SEQ ID NO:52 and SEQ ID NO:58 is 80%, the global percent identity between SEQ ID NO:52 and SEQ ID NO:60 is 75%. Based on the amino acid alignment in FIG. 6, there are three regions of high amino acid similarity among SEQ ID NO:52, 54, 56, 58 and 60. The first conserved domain, corresponding to the region between amino acid 19 through amino acid 161 in SEQ ID NO:52, is 73% identical between SEQ ID NO:52 and SEQ ID NO:54, 76% identical between SEQ ID NO:52 and SEQ ID NO:56, 83% identical between SEQ ID NO:52 and SEQ ID NO:58 and 78% identical between SEQ ID NO:52 and SEQ ID NO:60. The second conserved domain, corresponding to the region between amino acid 241 through amino acid 322 in SEQ ID NO:52, is 86% identical between SEQ ID NO:52 and SEQ ID NO:54, 83% identical between SEQ ID NO:52 and SEQ ID NO:56, 86% identical between SEQ ID NO:52 and SEQ ID NO:58 and 86% identical between SEQ ID NO:52 and SEQ ID NO:60. The third conserved domain, corresponding to the region between amino acid 376 through amino acid 466 in SEQ ID NO:52, is 81% identical between SEQ ID NO:52 and SEQ ID NO:54, 78% identical between SEQ ID NO:52 and SEQ ID NO:56, 82% identical between SEQ ID NO:52 and SEQ ID NO:58 and 77% identical between SEQ ID NO:52 and SEQ ID NO:60.

As disclosed in Example 2, expressing a GmZF_Glyma19g40220.1 transcript contained in vectors RTP4931-1 and RTP4932-1 results in reduced cyst counts when operably linked to a Super promoter and an AtTPP promoter and expressed in soybean roots. As disclosed in Example 1, the transcript contains an open reading frame with DNA sequence disclosed as SEQ ID NO: 61 and the amino acid sequence disclosed as SEQ ID NO:62. The DNA sequence described by SEQ ID NO:61 was used to identify a similar gene from soybean described by SEQ ID NO:63, with corresponding protein translation described by SEQ ID NO:64. The amino acid alignment to SEQ ID NO:62 is shown in FIG. 7.

As disclosed in Example 2, expressing a ZmAAA_ATPase transcript contained in vector RTP4453-1 results in reduced cyst counts when operably linked to a Super promoter and expressed in soybean roots. As disclosed in Example 1, the transcript contains an open reading frame with DNA sequence disclosed as SEQ ID NO:65 and the amino acid sequence disclosed as SEQ ID NO:66. The DNA sequence described by SEQ ID NO:65 was used to identify a similar gene from sorghum bicolor described by SEQ ID NO:67 with corresponding protein translation described by SEQ ID NO:68. The amino acid alignment to SEQ ID NO:66 is shown in FIG. 8.

As disclosed in Example 2, expressing a GmNPR1-like transcript contained in vector RTP4926-1 results in reduced cyst counts when operably linked to a AtTPP promoter and expressed in soybean roots. As disclosed in Example 1, the transcript contains an open reading frame with DNA sequence disclosed as SEQ ID NO:69 and the amino acid sequence disclosed as SEQ ID NO:70. The DNA sequence described by SEQ ID NO:69 was used to identify similar genes from other plant species, described by SEQ ID NO:71, 73, 75, 77, 79 and 81, with corresponding protein translations described by SEQ ID NO:72, 74, 76, 78, 80 and 82. The amino acid alignment to SEQ ID NO:70 is shown in FIG. 9a-c. The global percent identity between SEQ ID NO:70 and SEQ ID NO:72 is 74%, the global percent identity between SEQ ID NO:70 and SEQ ID NO:74 is 67%, the global percent identity between SEQ ID NO:70 and SEQ ID NO:76 is 67%, the global percent identity between SEQ ID NO:70 and SEQ ID NO:78 is 68%, the global percent identity between SEQ ID NO:70 and SEQ ID NO:80 is 67%, the global percent identity between SEQ ID NO:70 and SEQ ID NO:82 is 68%. Based on the amino acid alignment in FIG. 9, there are three regions of high amino acid similarity among SEQ ID NO:70, 72, 74, 76, 78, 80 and 82. The first conserved domain, corresponding to the region between amino acid 257 through amino acid 346 in SEQ ID NO:70, is 94% identical between SEQ ID NO:70 and SEQ ID NO:72, 91% identical between SEQ ID NO:70 and SEQ ID NO:74, 86% identical between SEQ ID NO:70 and SEQ ID NO:76, 86% identical between SEQ ID NO:70 and SEQ ID NO:78 90% identical between SEQ ID NO:70 and SEQ ID NO:80 and 88% identical between SEQ ID NO:70 and SEQ ID NO:82. The second conserved domain, corresponding to the region between amino acid 386 through amino acid 443 in SEQ ID NO:70, is 93% identical between SEQ ID NO:70 and SEQ ID NO:72, 86% identical between SEQ ID NO:70 and SEQ ID NO:74, 95% identical between SEQ ID NO:70 and SEQ ID NO:76, 91% identical between SEQ ID NO:70 and SEQ ID NO:78, 91% identical between SEQ ID NO:70 and SEQ ID NO:80 and 93% identical between SEQ ID NO:70 and SEQ ID NO:82. The third conserved domain, corresponding to the region between amino acid 470 through amino acid 517 in SEQ ID NO:70, is 83% identical between SEQ ID NO:70 and SEQ ID NO:72, 90% identical between SEQ ID NO:70 and SEQ ID NO:74, 90% identical between SEQ ID NO:70 and SEQ ID NO:76, 85% identical between SEQ ID NO:70 and SEQ ID NO:78 90% identical between SEQ ID NO:70 and SEQ ID NO:80 and 90% identical between SEQ ID NO:70 and SEQ ID NO:82.

1. A nematode-resistant transgenic plant transformed with an expression vector comprising an isolated polynucleotide encoding a polypeptide selected from the group consisting of
a) a transferase comprising amino acids 1 to 448 of SEQ ID NO:2; and
b) a zinc finger polypeptide selected from the group consisting of SEQ ID NO:62 and SEQ ID NO:64.

2. A seed which is true breeding for a transgene comprising at least one polynucleotide encoding a polypeptide selected from the group consisting of
a) a transferase comprising amino acids 1 to 448 of SEQ ID NO:2; and
b) a zinc finger polypeptide selected from the group consisting of SEQ ID NO:62 and SEQ ID NO:64,
wherein the transgene confers increased nematode resistance to the plant grown from the transgenic seed.

3. An expression vector comprising a promoter operably linked to a polynucleotide encoding at least one polypeptide selected from the group consisting of:
a) a transferase comprising amino acids 1 to 448 of SEQ ID NO:2; and
b) a zinc finger polypeptide selected from the group consisting of SEQ ID NO:62 and SEQ ID NO:64.

4. The expression vector of claim 3, wherein the promoter is a constitutive promoter.

5. The expression vector of claim 3, wherein the promoter is capable of specifically directing expression in plant roots.

6. The expression vector of claim 3, wherein the promoter is capable of specifically directing expression in a syncytia site of a plant infected with nematodes.

7. A method of producing a nematode-resistant transgenic plant, wherein the method comprises the steps of:
a) transforming a wild type plant cell with an expression vector comprising a promoter operably linked to a polynucleotide encoding a polypeptide selected from the group consisting of
i) a transferase comprising amino acids 1 to 448 of SEQ ID NO:2; and
ii) a zinc finger polypeptide selected from the group consisting of SEQ ID NO:62 and SEQ ID NO:64;
b) regenerating transgenic plants from the transformed plant cell; and
c) selecting transgenic plants for increased nematode resistance as compared to a control plant of the same species.
